(19) United States
(12) Patent Application Publication
(10) Pub. No.: US 2024/0206966 A1
(43) Pub. Date: Jun. 27, 2024

(54) CATHETER WITH ADJUSTABLE WORKING LENGTH CATHETER SHAFT

(71) Applicant: Medtronic Ireland Manufacturing Unlimited Company, Dublin 2 (IE)

(72) Inventors: Gary Kelly, Windsor, CA (US); Brian J. Castelli, Santa Rosa, CA (US); Brian Dowling, Santa Rosa, CA (US); Cian Michael Ryan, Santa Rosa, CA (US); Jaclyn N. Kawwas, Philadelphia, PA (US); Manoj Kumar Singh, Santa Rosa, CA (US)

(21) Appl. No.: 18/556,777

(22) PCT Filed: Apr. 26, 2022
(86) PCT No.: PCT/EP2022/061006

§ 371 Date: Oct. 23, 2023

Related U.S. Application Data
(60) Provisional application No. 63/179,857, filed on Apr. 26, 2021.
(60) Provisional application No. 63/179,877, filed on Apr. 26, 2021.
(60) Provisional application No. 63/179,865, filed on Apr. 26, 2021.

(51) Int. Cl. A61B 18/14; A61M 25/00; A61M 25/01

(52) U.S. Cl. CPC A61B 18/1492; A61M 25/0041; A61M 25/0043; A61M 25/0113; A61M 25/0136

(57) ABSTRACT

Various embodiments of catheters are disclosed. In one aspect, a catheter includes a catheter shaft actuator (e.g., a catheter handle) and a catheter shaft, where the working length of the catheter shaft is adjustable, and where this working length is defined between distal ends of the catheter shaft actuator and catheter shaft, respectively. In another aspect, a catheter includes a catheter handle and a shuttle to which a catheter shaft may be secured, with the shuttle being movable relative to at least a distal end of the catheter handle. In another aspect, a catheter includes a catheter shaft, a catheter shaft actuator that is movably mounted on the catheter shaft, and a housing, where the catheter shaft extends proximally to the housing, where a generator cable extends proximally from the housing, and where the catheter shaft actuator is disposable distally of the housing along the catheter shaft.

### FIELD

The present disclosure generally relates to the field of catheters (e.g., neuromodulation; denervation; diagnostic) and more particularly at least generally relates to the working length of the catheter shaft.

### BACKGROUND

At least some catheters have a catheter shaft that incorporates a non-linear catheter shaft section at an appropriate location along the length of the catheter shaft. For instance, some diagnostic catheters have a pigtail curve along a portion of the catheter shaft. Certain neuromodulation and/or renal denervation catheters utilize a catheter shaft with a distal end section having a helical or spiral configuration. One or more electrodes may be spaced along such a distal end section. A guide wire may be directed through a lumen of at least the distal end section of the catheter shaft. Such a guide wire retains the distal end section in a delivery configuration where the catheter shaft may be directed into/through the vasculature of a patient. Once the distal end section of the catheter shaft is in the desired location within the vasculature, the guide wire may be withdrawn from the distal end section of the catheter shaft such that it expands within the vasculature, typically into contact with an inner wall of the vasculature. Standalone catheter shaft straighteners exist to facilitate insertion of the guide wire into/through the distal end section of the catheter shaft, and are generally in the form of a tube.

A handle of a neuromodulation catheter is typically used to torque the catheter shaft and to advance the catheter shaft within the patient's vasculature. Torqueing and/or advancement of the catheter shaft may be undertaken during repositioning, within the vasculature, of a deployed distal end section of a catheter shaft of the above-noted type.

Options for introducing a catheter into the patient's vasculature include using a femoral access, a radial access, a brachial access, and the like. The distance between a typical femoral access and the renal artery is typically significantly less than the distance between a typical radial access and the same renal artery. As such, the same renal neuromodulation/denervation catheter is typically sold in two different configurations for these different vasculature accesses. The catheter shaft of a renal neuromodulation/denervation catheter for a radial access is typically longer than the catheter shaft of a renal neuromodulation/denervation catheter for a femoral access.

### SUMMARY

A first aspect of the present invention is directed to a catheter with an adjustable working length for its catheter shaft, with this “working length” being defined between a distal end of the catheter shaft and a structure (e.g., a catheter handle, or more generally a catheter shaft actuator) that can be used to manipulate the catheter shaft, such as torqueing the catheter shaft and/or advancing the catheter shaft relative to a patient's vasculature. Both the configuration of such an adjustable working length catheter and operational characteristics, operation, assembly, and manufacture of such an adjustable working length catheter are within the scope of this Summary. The adjustable working length catheter may be of any appropriate type and may provide any appropriate function or combination of functions when disposed in a patient's vasculature (e.g., diagnostic, therapeutic, neuromodulation, denervation). Various catheter shaft actuators and associated features/components are addressed in this Summary and may be used in relation to this first aspect.

A second aspect of the present invention is directed to what may be characterized as an adjustable catheter handle assembly. Adjustment of such a catheter handle assembly may be utilized to change a working length of a catheter shaft. In any case, the catheter handle assembly includes a catheter handle and a shuttle, where a catheter shaft may be mounted/secured to this shuttle. The shuttle is movable relative to at least a portion of the catheter handle, for instance relative to at least a distal end of the catheter handle (e.g., a working length of a catheter shaft extending distally from such a distal end) and including along a dimension corresponding with a spacing between this distal end of the catheter handle and a proximal end of the catheter handle. Various catheter handle assemblies and associated features/components are addressed in this Summary and may be used in relation to this second aspect.

A third aspect of the present invention is directed to a catheter having a catheter shaft, a catheter shaft actuator, and housing. The catheter shaft actuator is movably mounted on the catheter shaft and is movable relative to the housing. At least part of the catheter shaft actuator is disposed distally of this housing in at least one relative position between the catheter shaft actuator and the housing. The housing may be in the form of a conventional catheter handle, or may simply be in the form of an electrical components housing. Various catheters and associated features/components are addressed in this Summary and may be used in relation to this third aspect.

A number of additional aspects of the present invention will be addressed, and that may entail being able to dispose a catheter shaft clamp or the like in a clamped or clamping configuration relative to a catheter shaft associated with the actuator handle (e.g., a catheter handle). In such a case and unless otherwise noted herein to the contrary, this allows the actuator handle to be used to manipulate the catheter shaft in any appropriate manner and including while a distal end section of the catheter shaft is disposed within the vasculature of a patient. For instance, the actuator handle may be used to torque the catheter shaft, to advance the catheter shaft along the vasculature of the patient, or both.

An additional aspect of the present invention is directed to a catheter. The catheter includes a catheter handle, a catheter shaft, and a catheter shaft actuator (e.g., a structure that may be used to maneuver/manipulate the catheter shaft). The catheter shaft extends through the catheter shaft actuator and at least into the catheter handle. The catheter shaft actuator includes a body, a first tube that extends distally from this body, and a second tube that extends proximally from this body. The first tube and the second tube are disposed in spaced relation to one another (e.g., along a length dimension of the catheter shaft actuator), with a catheter shaft clamp being disposed between the first tube and the second tube (e.g., relative to a length dimension of the catheter shaft clamp). A clamping actuator is interconnectable with the catheter shaft clamp. The catheter shaft clamp is disposable in each of a clamped configuration (relative to the catheter shaft, and that maintains the catheter shaft actuator in a fixed position relative to this catheter shaft) and a released configuration (relative to the catheter shaft, and that allows the catheter shaft actuator to move along this catheter shaft). When the catheter shaft actuator is disposed in its proximal-most position relative to the catheter handle, the second tube of the catheter shaft actuator may extend into a distal end of the catheter handle.

An additional aspect of the present invention is directed to a catheter. The catheter includes a catheter shaft actuator (e.g., a structure that may be used to maneuver/manipulate a catheter shaft), a catheter shaft, and a housing. The catheter shaft extends through the catheter shaft actuator and at least into the housing. The catheter shaft actuator may be disposed on a distal side of the housing, and a generator cable may extend from a proximal end of this housing. The catheter shaft actuator may include a proximal housing section and a distal housing section. A perimeter of the distal housing section is larger (e.g., a larger effective outer diameter) than a perimeter of the proximal housing section. A clamping actuator is interconnectable with a catheter shaft clamp. The catheter shaft clamp is disposable in each of a clamped configuration (relative to the catheter shaft, and that maintains the catheter shaft actuator in a fixed position relative to this catheter shaft) and a released configuration (relative to the catheter shaft, and that allows the catheter shaft actuator to move along this catheter shaft). The clamping actuator and the catheter shaft clamp may be incorporated by the noted distal housing section of the catheter shaft actuator. When the catheter shaft actuator is disposed in its proximal-most position relative to the housing, at least a distal end section of the housing may extend into a proximal end of the catheter shaft actuator (e.g., a majority of the housing could be disposed within the catheter shaft actuator).

An additional aspect of the present invention is directed to a catheter. The catheter includes a catheter shaft actuator (e.g., a structure that may be used to maneuver/manipulate a catheter shaft), a catheter shaft, and a catheter handle. The catheter shaft extends through the catheter shaft actuator and at least into the catheter handle. The catheter shaft actuator may be disposed on a distal end of the catheter handle, and a generator cable may extend from a proximal end of this catheter handle. The catheter shaft actuator may include a proximal housing section and a distal housing section. A perimeter of the distal housing section is larger (e.g., a larger effective outer diameter) than a perimeter of the proximal housing section. A clamping actuator is interconnectable with a catheter shaft clamp. The catheter shaft clamp is disposable in each of a clamped configuration (relative to the catheter shaft, and that maintains the catheter shaft actuator in a fixed position relative to this catheter shaft) and a released configuration (relative to the catheter shaft, and that allows the catheter shaft actuator to move along this catheter shaft). The clamping actuator and the catheter shaft clamp may be incorporated by the noted distal housing section of the catheter shaft actuator. When the catheter shaft actuator is disposed in its proximal-most position relative to the catheter handle, a proximal end section of the catheter shaft actuator (e.g., the above-noted proximal housing section) may extend into a distal end of the catheter shaft actuator.

An additional aspect of the present invention is directed to a catheter. The catheter includes a catheter shaft, an actuator or catheter handle (e.g., a catheter shaft actuator—a structure that may be used to maneuver/manipulate a catheter shaft), and a housing. The catheter shaft extends through the handle and into the housing. A clamping actuator and a catheter shaft clamp are each interconnectable with or incorporated by the handle. The catheter shaft clamp is disposable in each of a clamped configuration (relative to the catheter shaft, and that maintains the handle in a fixed position relative to this catheter shaft) and a released configuration (relative to the catheter shaft, and that allows the handle to move along this catheter shaft). The clamping actuator and the catheter shaft clamp may be of any of the configurations addressed herein for use in combination with this aspect (e.g., a rotary catheter shaft clamp). When the catheter shaft actuator is disposed in its proximal-most position relative to the handle: 1) a distal end section of the housing may extend into a proximal end of the handle (including where a majority of the housing is proximally disposed relative to the handle); or 2) a majority of the housing may be disposed within the handle, including where a proximal end section of the housing extends proximally from a proximal end of the handle.

An additional aspect of the present invention is directed to a catheter. The catheter includes a catheter shaft, an actuator handle (e.g., a catheter shaft actuator—a structure that may be used to maneuver/manipulate the catheter shaft), and a housing. The catheter shaft extends through the handle and into the housing, and the handle and housing may be spaced form on another along the catheter shaft. The actuator handle includes catheter shaft clamp, and a clamping actuator is at least operatively interconnected with this catheter shaft clamp. The catheter shaft clamp is disposable in each of a clamped configuration (relative to the catheter shaft, and that maintains the actuator handle in a fixed position relative to this catheter shaft) and a released configuration (relative to the catheter shaft, and that allows the actuator handle to move along the catheter shaft). The clamping actuator is operable to at least dispose the catheter shaft clamp in its released configuration. In one embodiment, the clamping actuator is in the form of a collet. In one embodiment, the clamping actuator is moved in a first dimension (e.g., parallel to the portion of the catheter shaft extending through the handle) to at least reduce its engagement with the catheter shaft clamp (e.g., the catheter shaft clamp being movable in a second dimension, including where this second dimension is orthogonal to the noted first dimension). A biasing member(s) may engage the clamping actuator to engage the catheter shaft clamp to dispose the catheter shaft clamp in the clamped configuration (e.g., the biasing member(s) may bias the clamping actuator in a direction that is opposite that in which the clamping actuator is moved to dispose the catheter shaft clamp in its clamped configuration). In one embodiment, the clamping actuator includes an actuation section and a clamping section that are disposed in a fixed position relative to one another. The actuation section may be moved in a first dimension to move the clamping section in this same first dimension and into a released configuration (e.g., the first dimension may be orthogonal to a length dimension of the portion of the catheter shaft within the handle). A biasing member(s) may engage the clamping section to bias the clamping section into engagement with the catheter shaft to provide clamped configuration relative to the catheter shaft).

An additional aspect of the present invention is directed to a telescoping catheter handle. The catheter handle includes a primary handle section and at least one handle extension that telescopes relative to the primary handle section (e.g., each handle extension telescoping in a proximal direction relative to at least the primary handle section; each handle extension moving axially relative to at least the primary handle section). Each telescoping handle extension may be disposed in at least one fixed position relative to at least the primary handle section. A shuttle may be fixed to one handle extension of the catheter handle (e.g., a proximal-most of the handle extensions), and a catheter shaft may be fixed/attached to this shuttle. Telescoping the catheter handle may then be used to change a working length of the catheter shaft. The catheter handle may include a catheter shaft clamp and a clamping actuator that is at least operatively interconnected with the catheter shaft clamp. The catheter shaft clamp is disposable in each of a clamped configuration (relative to the catheter shaft, and that maintains the catheter handle in a fixed position relative to this catheter shaft) and a released configuration (relative to the catheter shaft, and that allows the catheter handle to move along the catheter shaft). The clamping actuator may be used to dispose the catheter shaft clamp in at least one of its clamped configuration or its released configuration.

An additional aspect of the present invention is directed to an actuator handle (e.g., a structure that may be used to maneuver/manipulate a catheter shaft; a catheter handle). A shuttle is movable along a length dimension of the actuator handle, and a catheter shaft may be mounted/fixed to this shuttle (e.g., the actuator handle may be moved relative to each of the catheter shaft and the shuttle). Moving the actuator handle relative the shuttle may be used to change a working length of the catheter shaft that extends distally of the actuator handle. A proximal shaft may extend proximally from a proximal end of the actuator handle, a distal shaft may extend distally from a distal end of this actuator handle, or both. The shuttle may be disposed within the proximal shaft and may be maintained in a fixed position relative to the proximal shaft at one or more locations along the length of the proximal shaft (e.g., by disposing a detent in a corresponding hole through a wall of the proximal shaft). Similarly, the shuttle may be disposed within the distal shaft and may be maintained in a fixed position relative to the distal shaft at one or more locations along the length of the distal shaft (e.g., disposing a detent in a corresponding hole through a wall of the distal shaft). The actuator handle may include a catheter shaft clamp and a clamping actuator that is at least operatively interconnected with the catheter shaft clamp. The catheter shaft clamp is disposable in each of a clamped configuration (relative to the catheter shaft, and that maintains the actuator handle in a fixed position relative to this catheter shaft) and a released configuration (relative to the catheter shaft, and that allows the actuator handle to move along the catheter shaft). The clamping actuator may be used to dispose the catheter shaft clamp in at least one of its clamped configuration or its released configuration.

An additional aspect of the present invention is directed to an actuator handle (e.g., a structure that may be used to maneuver/manipulate a catheter shaft; a catheter handle). A shuttle is disposed within an interior of the actuator handle and is movable along a length dimension of the actuator handle. A catheter shaft may be mounted/fixed to this shuttle (e.g., the actuator handle may be moved relative to each of the catheter shaft and the shuttle). Moving the actuator handle relative the shuttle may be used to change a working length of the catheter shaft that extends distally of the actuator handle. A slot extends through a wall thickness of the actuator handle, and extends from a first slot end to a second slot end (e.g., the spacing between the first slot end and the second slot end defining a length dimension of the slot). The length dimension of the slot may at least generally correspond with a length dimension of the actuator handle. In any case, a knob or the like (more generally an actuator) is accessible on an exterior of the actuator handle and is interconnected with the shuttle. The knob may be actuated to reposition the shuttle along the slot. The ends of the slot may each include a recessed aperture in which the knob may be seated to lock the position of the knob relative to the actuator handle relative to the length dimension of the slot. The actuator handle may include a catheter shaft clamp and a clamping actuator that is at least operatively interconnected with the catheter shaft clamp. The catheter shaft clamp is disposable in each of a clamped configuration (relative to a catheter shaft when interconnected with the shuttle, and that maintains the actuator handle in a fixed position relative to this catheter shaft) and a released configuration (relative to a catheter shaft when interconnected with the shuttle, and that allows the actuator handle to move along the catheter shaft). The clamping actuator may be used to dispose the catheter shaft clamp in at least one of its clamped configuration or its released configuration.

An additional aspect of the present invention is directed to an actuator handle (e.g., a structure that may be used to maneuver/manipulate a catheter shaft; a catheter handle). A proximal shaft or sleeve extends proximally from a proximal end of the actuator handle (i.e., the actuator handle and proximal sleeve are collectively movable). A catheter shaft extends through both the actuator handle and the proximal sleeve. A stop is fixed or attached to a portion of the catheter shaft within the proximal sleeve. First and second stops are spaced along a length dimension of the actuator handle/proximal sleeve. Collectively moving the actuator handle and proximal sleeve in one direction relative to/along the catheter shaft will dispose the stop (catheter shaft) in engagement with the first stop to provide a first working length configuration for the catheter shaft. Moving the actuator handle and proximal sleeve in the opposite direction along/relative to the catheter shaft will dispose the stop (catheter shaft) in engagement with the second stop to provide a second working length configuration for the catheter shaft, where the first and second working length configurations are different. The first stop may be a proximal end of the actuator handle (to provide a longer working length configuration for the catheter shaft), and the second end may be a proximal end of the proximal sleeve (to provide a shorter working length configuration for the catheter shaft). The actuator handle may include a catheter shaft clamp and a clamping actuator that is at least operatively interconnected with the catheter shaft clamp. The catheter shaft clamp is disposable in each of a clamped configuration (relative to a catheter shaft when interconnected with the shuttle, and that maintains the actuator handle in a fixed position relative to this catheter shaft) and a released configuration (relative to a catheter shaft when interconnected with the shuttle, and that allows the actuator handle to move along the catheter shaft). The clamping actuator may be used to dispose the catheter shaft clamp in at least one of its clamped configuration or its released configuration.

An additional aspect of the present invention is directed to an actuator handle (e.g., a structure that may be used to maneuver/manipulate a catheter shaft; a catheter handle). A proximal shaft or sleeve extends proximally from a proximal end of the actuator handle (i.e., the actuator handle and proximal sleeve are collectively movable). A catheter shaft extends through both the actuator handle and the proximal sleeve. A first stop is fixed or attached to a portion of the catheter shaft within the proximal sleeve, and a second stop is fixed or attached to a portion of the catheter shaft that is disposed proximally of a proximal end of the proximal sleeve (the first and second stops, along with the portion of the catheter shaft disposed therebetween, may be referred to herein as a “shuttle”). At least one stop is incorporated by the proximal sleeve. Collectively moving the actuator handle and proximal sleeve in one direction relative to/along the catheter shaft will dispose the first stop (catheter shaft) in engagement with at least one stop of the proximal sleeve to define a first working length configuration for the catheter shaft. Moving the actuator handle and proximal sleeve in the opposite direction along/relative to the catheter shaft will dispose the second stop (catheter shaft) in engagement with at least one stop of proximal sleeve to define a second working length configuration for the catheter shaft, where the first and second working length configurations are different. The first stop (catheter shaft) may engage in interior side of a proximal end of the proximal sleeve, while the second stop (catheter shaft), may engage an exterior side of this same proximal end of the proximal sleeve (e.g., the “at least one stop” may be the proximal end of the proximal sleeve, with the noted first and second stops being larger than the aperture extending through the proximal end of the proximal sleeve). The actuator handle may include a catheter shaft clamp and a clamping actuator that is at least operatively interconnected with the catheter shaft clamp. The catheter shaft clamp is disposable in each of a clamped configuration (relative to a catheter shaft when interconnected with the shuttle, and that maintains the actuator handle in a fixed position relative to this catheter shaft) and a released configuration (relative to a catheter shaft when interconnected with the shuttle, and that allows the actuator handle to move along the catheter shaft). The clamping actuator may be used to dispose the catheter shaft clamp in at least one of its clamped configuration or its released configuration.

An additional aspect of the present invention is directed to an actuator handle (e.g., a structure that may be used to maneuver/manipulate a catheter shaft; a catheter handle). The actuator handle is movable relative to a first shaft, with a catheter shaft extending at least distally from this first shaft and including where at least part of the first shaft is disposable within the actuator handle. The first shaft may include a plurality of recesses or notches on its outer perimeter and that are spaced along a length dimension of the first shaft. The actuator handle may include a detent that is disposable within a given recess when aligned therewith (e.g., by moving the actuator handle relative to this first shaft). Disposing the detent within a given recess should maintain the first shaft (and thereby the catheter shaft) in a fixed position relative to the actuator handle. This may be referred to as a clamped configuration (of the first shaft relative to the actuator handle; of the catheter shaft relative to the actuator handle, as the catheter shaft extends distally from the first shaft). The detent may be biased toward such a clamped configuration. An actuator (accessible on an exterior of the actuator handle) may be activated to direct the detent out of a given recess in the first shaft, and that may referred to as a released configuration (of the first shaft relative to the actuator handle; of the catheter shaft relative to the actuator handle, as the catheter shaft extends distally from the first shaft). When the first shaft is in a released configuration relative to the actuator handle, the actuator handle may be moved relative to the first shaft and that will in turn change the working length of the catheter shaft. Instead of having a plurality of individual recesses that are spaced along the length dimension of the first shaft, the first shaft could instead utilize an elongate recess on the outer perimeter of the first shaft, and noted detent may engage the first shaft anywhere along the length of this elongate recess, and with a sufficient force, so as to dispose the first shaft in a clamped configuration relative to the actuator handle.

Further disclosed herein is a catheter that includes a catheter shaft actuator (e.g., a catheter handle) and a catheter shaft, where the working length of the catheter shaft is adjustable, and where this working length is defined between distal ends of the catheter shaft actuator and catheter shaft, respectively; as well as a catheter that includes a catheter handle and a shuttle to which a catheter shaft may be secured, with the shuttle being movable relative to at least a distal end of the catheter handle, as well as a catheter that includes a catheter shaft, a catheter shaft actuator that is movably mounted on the catheter shaft, and a housing, where the catheter shaft extends proximally to the housing, where a generator cable extends proximally from the housing, and where the catheter shaft actuator is disposable distally of the housing along the catheter shaft.

A number of feature refinements and additional features are applicable to the present invention. These feature refinements and additional features may be used individually or in any combination.

Various aspects of the present disclosure are also addressed by the following paragraphs and in the noted combinations:

1. A catheter, comprising:
a catheter shaft comprising a shaft distal end; and
a catheter shaft actuator comprising an actuator distal end, wherein said catheter shaft extends distally of said actuator distal end, and wherein a distance between said shaft distal end and said actuator distal end is adjustable.

2. The catheter of paragraph 1, further comprising:
a first configuration comprising a first distance between said actuator distal end and said shaft distal end; and
a second configuration comprising a second distance between said actuator distal end and said shaft distal end, wherein said first distance and said second distance are different.

3. The catheter of any of paragraphs 1-2, wherein said catheter shaft actuator is configured to at least one of apply torque to said catheter shaft and advance said catheter shaft relative to a vasculature of a patient.

4. The catheter of any of paragraphs 1-3, further comprising a multi-function catheter shaft tool comprising:
a tool distal end;
a tool proximal end;
a lumen that extends from said tool distal end to said tool proximal end, wherein said lumen is sized to accommodate said catheter shaft;
a catheter shaft straightener comprising said tool distal end;
said catheter shaft actuator; and
a catheter shaft clamp disposable in each of a clamping configuration and a released configuration relative to said lumen;
wherein said catheter shaft straightener extends proximally from said tool distal end and at least to said actuator distal end, and wherein said catheter shaft straightener and said catheter shaft actuator are configured to be collectively rotatable.

5. The catheter of paragraph 4, wherein said multi-function catheter shaft tool further comprises a clamping actuator operatively interconnected with said catheter shaft clamp.

6. The catheter of any of paragraphs 1-3, further comprising:
a housing, wherein said catheter shaft extends at least to a distal end of said housing;
a generator cable extending from said housing;
a catheter shaft clamp; and
a clamping actuator operatively connected with said catheter shaft clamp, wherein said clamping actuator is movable between at last two different positions to change said catheter shaft clamp between a released configuration relative to said catheter shaft and a clamping configuration relative to said catheter shaft;
wherein said catheter shaft actuator comprises said catheter shaft clamp and said clamping actuator, and wherein said catheter shaft actuator is movable along said catheter shaft and relative to each of said catheter shaft and said housing when said catheter shaft clamp is in said released configuration.

7. The catheter of paragraph 6, wherein at least substantially an entirety of said catheter shaft actuator is disposed distally of said housing when said catheter shaft actuator is disposed in a proximal-most position relative to said catheter shaft.

8. The catheter of paragraph 7, wherein said catheter shaft actuator comprises a proximal housing section and a distal housing section, wherein a perimeter of said distal housing section is larger than said proximal housing section, and wherein said distal housing section comprises said clamping actuator and said catheter shaft clamp.

9. The catheter of paragraph 6, wherein said catheter shaft actuator comprises an actuator handle.

10. The catheter of any of paragraphs 6 and 9, wherein a body of said catheter shaft actuator comprises at least one curved surface proceeding along a length dimension of said body.

11. The catheter of paragraph 6, wherein a proximal end section of said catheter shaft actuator is disposed within a distal end section of said housing when said catheter shaft actuator is disposed in a proximal-most position relative to said catheter shaft.

12. The catheter of paragraph 11, wherein said catheter shaft actuator comprises a proximal housing section and a distal housing section, wherein a perimeter of said distal housing section is larger than said proximal housing section, and wherein said distal housing section comprises said clamping actuator and said catheter shaft clamp.

13. The catheter of paragraph 12, wherein at least substantially an entirety of said proximal housing section of said catheter shaft actuator is disposed within said distal end section of said housing when said catheter shaft actuator is disposed in a proximal-most position relative to said catheter shaft.

14. The catheter of paragraph 6, wherein a majority of said housing is disposed within a proximal end section of said catheter shaft actuator when said catheter shaft actuator is disposed in a proximal-most position relative to said catheter shaft.

15. The catheter of paragraph 14, wherein said catheter shaft actuator comprises a handle.

16. The catheter of paragraph 6, wherein a distal end section of said housing is disposed within a proximal end section of said catheter shaft actuator when said catheter shaft actuator is disposed in a proximal-most position relative to said catheter shaft.

17. The catheter of paragraph 16, wherein a majority of said housing is disposed proximally of said catheter shaft actuator when said catheter shaft actuator is disposed in a proximal-most position relative to said catheter shaft.

18. The catheter of any of paragraphs 16-17, wherein a body of said catheter shaft actuator comprises at least one curved surface proceeding along a length dimension of said body.

19. The catheter of any of paragraphs 1-3, further comprising a catheter handle, wherein said catheter shaft extends at least to a distal end of said catheter handle, wherein at least part of said catheter shaft actuator is disposed distally of said catheter handle, and wherein said catheter shaft actuator comprises:
a passage extending through said catheter shaft actuator from said actuator distal end to an actuator proximal end, wherein said catheter shaft extends entirely through said catheter shaft actuator;
a clamping actuator on an exterior of said catheter shaft actuator;
a catheter shaft clamp operatively interconnected with said clamping actuator;
a clamping configuration, relative to said catheter shaft, when said clamping actuator is in a first position; and
a released configuration, relative to said catheter shaft, when said clamping actuator is in a second position, wherein said clamping actuator is movable between said first position and said second position to dispose said clamp in said clamping configuration and said released configuration, respectively.

20. The catheter of paragraph 19, wherein said catheter shaft actuator is movable along said catheter shaft and relative to each of said catheter shaft and said catheter handle when said clamping actuator is in said first position.

21. The catheter of any of paragraphs 19-20, wherein said catheter shaft actuator further comprises:
a body;
a first tube extending distally from said body, wherein said catheter shaft extends through said first tube;
a second tube extending proximally from said body, wherein said first tube and said second tube are spaced along a length of said catheter shaft actuator, and wherein said passage comprises an interior of said first tube and an interior of said second tube.

22. The catheter of paragraph 21, wherein said second tube extends into a distal end of said catheter handle when said catheter is disposed in one catheter shaft working length configuration.

23. The catheter of any of paragraphs 21-22, wherein said second tube is spaced from an entirety of said catheter handle when said catheter is disposed in another catheter shaft working length configuration.

24. The catheter of any of paragraphs 21-23, wherein said catheter shaft clamp is located in a space between said first tube and said second tube within said body.

25. The catheter of any of paragraphs 21-24, wherein said clamping actuator is disposed on an exterior of said body.

26. The catheter of any of paragraphs 19-20, wherein said catheter shaft actuator further comprises a body, wherein said body extends into a distal end of said catheter handle when said catheter is disposed in one catheter shaft working length configuration.

27. The catheter of paragraph 26, wherein said catheter shaft actuator is movable along said catheter shaft and relative to each of said catheter shaft and said catheter handle when said clamping actuator is in said first position.

28. The catheter of any of paragraphs 26-27, wherein said body of said catheter shaft actuator comprises a proximal housing section and a distal housing section, wherein a perimeter of said distal housing section is larger than said proximal housing section, wherein said distal housing section comprises said clamping actuator and said catheter shaft clamp, and wherein said proximal housing section is disposed within said catheter shaft handle when said catheter is disposed in said one configuration.

29. The catheter of any of paragraphs 26-28, wherein an entirety of said catheter shaft actuator is spaced from said catheter handle, along said catheter shaft, when said catheter is disposed in another catheter shaft working length configuration.

30. The catheter of any of paragraphs 1-3, wherein said catheter shaft actuator comprises an actuator handle and a clamping actuator connectable with said actuator handle, wherein said actuator handle comprises an actuation surface, wherein said clamping actuator comprises a clamp, wherein said clamp extends into said actuator handle and interfaces with said actuation surface, and wherein said catheter shaft extends through said clamp of said clamping actuator and through said actuator handle.

31. The catheter of paragraph 30, wherein said clamping actuator is threadably connected to said actuator handle.

32. The catheter of any of paragraphs 30-31, wherein said clamping actuator is disposed on a distal end section of said actuator handle.

33. The catheter of any of paragraphs 30-32, wherein axially moving said clamping actuator relative said actuator handle causes said actuation surface of said actuator handle to compress said clamp of said clamping actuator into clamping engagement with said catheter shaft.

34. The catheter of paragraph 33, wherein said clamping actuator is rotatably connected with said actuator handle, and wherein rotating said clamping actuator relative to said actuator handle also moves said clamping actuator axially relative to said actuator handle.

35. The catheter of any of paragraphs 30-34, further comprising:
a housing, wherein said catheter shaft extends at least to a distal end of said housing; and
a generator cable extending from said housing;
wherein at least a distal end section of said housing is disposed within a proximal end section of said actuator handle when said catheter shaft actuator is disposed in a proximal-most position relative to said catheter shaft.

36. The catheter of paragraph 35, wherein a majority of said housing is disposed within said actuator handle when said catheter shaft actuator is disposed in a proximal-most position relative to said catheter shaft.

37. The catheter of any of paragraphs 1-3, wherein said catheter shaft actuator comprises an actuator handle and a clamping actuator connectable with said actuator handle, wherein said actuator handle comprises a clamp, wherein said clamping actuator comprises an actuation surface, wherein said clamp of said actuator handle extends into said clamping actuator and interfaces with said actuation surface, and wherein said catheter shaft extends through said clamping actuator and through said clamp of said actuator handle.

38. The catheter of paragraph 37, wherein said clamping actuator is threadably connected to said handle.

39. The catheter of any of paragraphs 37-38, wherein said clamping actuator is disposed on a distal end section of said actuator handle.

40. The catheter of any of paragraphs 37-39, wherein axially moving said clamping actuator relative said actuator handle causes said actuation surface of said clamping actuator to compress said clamp of said actuator handle into clamping engagement with said catheter shaft.

41. The catheter of paragraph 40, wherein said clamping actuator is rotatably connected with said actuator handle, and wherein rotating said clamping actuator relative to said actuator handle also moves said clamping actuator axially relative to said actuator handle.

42. The catheter of any of paragraphs 1-3, wherein said catheter shaft actuator comprises an actuator handle, wherein said actuator handle comprises first and second clamping segments that are separated by a space, wherein said catheter shaft extends through said space between said first and second clamping segments, and wherein said actuator handle further comprises a clamping actuator engageable with said first and second clamping segments.

43. The catheter of paragraph 42, wherein said first and second clamping segments are biased toward a clamping configuration relative to said catheter shaft, and wherein moving said clamping actuator relative to said first and second clamping segments disposes said first and second clamping segments in a released configuration relative to said catheter shaft.

44. The catheter of paragraph 43, wherein said clamping actuator is movable between said first and second positions, wherein first and second clamping segments are in said clamping configuration when said clamping actuator is in said first position and where said first and second clamping segments are separated by a first space, and wherein said first and second clamping segments are in said released configuration when said clamping actuator is in said second position and where said first and second clamping segments are separated by a second space to allow said catheter shaft actuator to move along and relative to said catheter shaft, wherein said second space is larger than said first space.

45. The catheter of paragraph 44, wherein said clamping actuator is biased toward said first position.

46. The catheter of any of paragraphs 1-3, wherein said catheter shaft actuator comprises an actuator handle, a clamping actuator, and a rotary catheter shaft clamp comprising a first clamping segment and a second clamping segment.

47. The catheter of paragraph 46, wherein said first clamping segment and said second clamping segment are engageable with opposite sides of said catheter shaft.

48. The catheter of any of paragraphs 46-47, further comprising a first biasing member engaged with said first clamping segment on a first side of an axis about which said rotary catheter shaft clamp is rotatable, and wherein said clamping actuator is engageable with said second clamping segment on an opposite second side of said axis.

49. The catheter of paragraph 48, wherein said first biasing member simultaneously biases said first clamping segment and said second clamping segment into a clamped configuration for said catheter shaft clamp relative to said catheter shaft, and wherein moving said clamping actuator relative to said first clamping segment disposes said catheter shaft clamp in a released configuration relative to said catheter shaft to allow said catheter shaft actuator to move along and relative to said catheter shaft.

50. The catheter of paragraph 49, wherein said clamping actuator is biased to a position where said catheter shaft clamp is disposed in said clamped configuration relative to said catheter shaft.

51. The catheter of any of paragraphs 46-50, further comprising:
a housing, wherein said catheter shaft extends at least to a distal end of said housing; and
a generator cable extending from said housing;
wherein at least a distal end section of said housing is disposed within a proximal end section of said actuator handle when said catheter shaft actuator is disposed in a proximal-most position relative to said catheter shaft.

52. The catheter of paragraph 51, wherein a majority of said housing is disposed proximally beyond said actuator handle when said catheter shaft actuator is disposed in a proximal-most position relative to said catheter shaft.

53. The catheter of any of paragraphs 1-3, wherein said catheter shaft actuator comprises an actuator handle and a clamping actuator, wherein said clamping actuator comprises a collet rotatably connected with an outer perimeter of said actuator handle, wherein said catheter shaft extends through each of said actuator handle and said clamping actuator, wherein said collet is rotatable between at least first and second positions relative to said actuator handle, wherein said actuator handle is disposed in a clamping configuration, relative to said catheter shaft, when said collet is in said first position, and wherein said actuator handle is disposed in a released configuration, relative to said catheter shaft, when said collet is in said second position.

54. The catheter of any of paragraphs 1-3, wherein said catheter shaft actuator comprises an actuator handle, a first clamp, and a clamping actuator, wherein said catheter shaft extends through said actuator handle, wherein said first clamp is movable along a first axis relative to said catheter shaft between a clamping configuration and a released configuration, wherein said clamping actuator is movable along a second axis between at least first and second positions to dispose said first clamp in said clamping configuration and said released configuration, respectively, wherein said first clamp clamps against said catheter shaft in said clamping configuration, and wherein said catheter shaft actuator is movable along and relative to said catheter shaft when said first clamp is in said released configuration.

55. The catheter of paragraph 54, wherein said second axis is orthogonal to said catheter shaft.

56. The catheter of any of paragraphs 54-55, wherein said first axis is parallel to said catheter shaft.

57. The catheter of any of paragraphs 54-56, further comprising a biasing member disposed between said clamping actuator and said first clamp when said clamping actuator is in said first position.

58. The catheter of any of paragraphs 1-3, wherein said catheter shaft actuator comprises an actuator handle, a first clamp, a clamping actuator interconnected with said first clamp, and a biasing member that engages at least one of said first clamp and said clamping actuator, wherein said catheter shaft extends through said actuator handle, wherein said clamping actuator is movable between at least first and second positions and said first clamp is movable between a clamping configuration and a released configuration, relative to said catheter shaft, wherein one of said clamping actuator and said biasing member disposes said first clamp in said clamping configuration, and wherein the other of said clamping actuator and said biasing member disposes said first clamp in said released configuration.

59. The catheter of paragraph 58, wherein said first clamp moves in a first direction relative to said actuator handle in moving between said clamping configuration and said released configuration.

60. The catheter of paragraph 59, wherein said first direction is orthogonal to said a length dimension of catheter shaft.

61. The catheter of any of paragraphs 58-60, wherein said biasing member disposes said first clamp in said clamping configuration when said clamping actuator is in said first position, and wherein moving said clamping actuator from said first position to said second position compresses said biasing member and disposes said first clamp in said released configuration.

62. The catheter of any of paragraphs 53-61, further comprising:
a housing, wherein said catheter shaft extends at least to a distal end of said housing; and
a generator cable extending from said housing, and wherein said catheter shaft actuator is movable along said catheter shaft relative to each of said catheter shaft and said housing.

63. The catheter of paragraph 62, wherein said housing is proximally spaced from said actuator handle.

64. The catheter of any of paragraphs 1-3, further comprising:
a telescoping catheter handle.

65. The catheter of paragraph 64, further comprising:
a first catheter handle configuration where said telescoping catheter handle is of a first handle length and said catheter shaft is of a first working length; and
a second catheter handle configuration where said telescoping catheter handle is of a second handle length and said catheter shaft is of a second working length;
wherein said first handle length is different from said second handle length and said first working length is different from said second working length.

66. The catheter of paragraph 65, wherein each of said first catheter handle configuration and said second catheter handle configuration are pre-set.

67. The catheter of any of paragraphs 65-66, wherein said telescoping catheter handle comprises first and second holes that are spaced along a length dimension of said telescoping catheter handle and that correspond with said first catheter handle configuration and said second catheter handle configuration, respectively, said catheter further comprising at least one detent, wherein said at least one detent is disposed in in said first hole when said telescoping catheter handle is in said first catheter handle configuration, and wherein said at least one detent is disposed in said second hole when said telescoping catheter handle is in said second catheter handle configuration.

68. The catheter of paragraph 67, wherein said at least one detent is biased to a locking position.

69. The catheter of any of paragraphs 64-68, further comprising a catheter shaft clamp disposable between a clamping configuration relative to said catheter shaft and a released configuration relative to said catheter shaft.

70. The catheter of paragraph 69, wherein said catheter shaft clamp is disposed on a distal end of said telescoping catheter handle.

71. The catheter of any of paragraphs 64-70, wherein said telescoping catheter handle telescopes in a proximal direction.

72. The catheter of any of paragraphs 62-71, further comprising a generator connector on a proximal end of said telescoping catheter handle.

73. The catheter of any of paragraphs 1-3, wherein said catheter shaft actuator comprises an actuator handle, said catheter further comprising:
a shuttle movable along a length of said actuator handle between at least first and second positions that are spaced along said length of said actuator handle, wherein said catheter shaft is interconnected with said shuttle such that said shuttle and said catheter shaft are collectively movable relative to said actuator handle at least generally in an axial dimension.

74. The catheter of paragraph 73, wherein said shuttle is disposed within an interior of said actuator handle.

75. The catheter of any of paragraphs 73-74, wherein said first and second positions are pre-set.

76. The catheter of any of paragraphs 73-75, wherein disposing said shuttle in said first position provides a first working length for said catheter shaft and disposing said shuttle in said second position provides a second working length for said catheter shaft, wherein said first working length and said second working length are different.

77. The catheter of any of paragraphs 73-76, wherein said shuttle comprises a detent that is biased toward a perimeter wall of said actuator handle, wherein said perimeter wall comprises first and second apertures, wherein said detent protrudes through said first aperture and defines said first position when said detent is aligned with said first aperture, and wherein said detent protrudes through said second aperture and defines said second position when said detent is aligned with said second aperture.

78. The catheter of any of paragraphs 73-76, wherein said actuator handle comprises a perimeter wall and a slot extending through said perimeter wall and extending between a first slot end and a second slot end, and wherein said first slot end defines said first position and said second slot end defines said second position.

79. The catheter of paragraph 78, further comprising a lock interconnected with said shuttle and disposed on an exterior of said actuator handle, wherein said lock is separately disposable in a locked configuration and an unlocked configuration, wherein said shuttle is movable relative to said actuator handle when said lock is in said unlocked configuration, and wherein a position of said shuttle is fixed relative to said actuator handle when said lock is in said locked configuration.

80. The catheter of paragraph 79, wherein said lock is biased toward said locked configuration.

81. The catheter of any of paragraphs 79-80, wherein said lock is movable away from said perimeter wall of said catheter shaft handle to dispose said lock in said unlocked configuration.

82. The catheter of any of paragraphs 79-81, wherein said lock comprises a knob.

83. The catheter of any of paragraphs 78-82, wherein said first slot end defines one catheter shaft working length configuration and said second slot end defines a different catheter shaft working length configuration.

84. The catheter of any of paragraphs 73-77, further comprising:
a proximal shaft extending proximally from a proximal end of said actuator handle; and
a distal shaft extending distally from a distal end of said actuator handle;
wherein said catheter shaft extends through said distal shaft and said shuttle is separately disposable in each of said proximal shaft and said distal shaft.

85. The catheter of paragraph 84, wherein said proximal shaft and said distal shaft are of different lengths.

86. The catheter of any of paragraphs 84-85, wherein said proximal shaft is longer than said distal shaft.

87. The catheter of any of paragraphs 84-86, wherein said proximal shaft comprises a first aperture that extends through a perimeter wall of said proximal shaft and that corresponds with said first position, and wherein said distal shaft comprises a second aperture that extends through a perimeter wall of said distal shaft and that corresponds with said second position.

88. The catheter of paragraph 87, wherein said shuttle comprises a detent that is biased toward said perimeter wall of said proximal shaft when said shuttle is disposed within said proximal shaft, wherein said detent is biased toward said perimeter wall of said distal shaft when said shuttle is disposed within said distal shaft, wherein said detent protrudes through said first aperture and defines said first position when said detent is aligned with said first aperture, and wherein said detent protrudes through said second aperture and defines said second position when said detent is aligned with said second aperture.

89. The catheter of any of paragraphs 87-88, wherein said first aperture defines one catheter shaft working length configuration and said second aperture defines a different catheter shaft working length configuration.

90. The catheter of any of paragraphs 1-3, further comprising:
an actuator handle;
a proximal sleeve extending proximally from a proximal end of said actuator handle, wherein said catheter shaft through each of said actuator handle and said proximal sleeve;
a first stop; and
a second stop;
wherein said first stop and said second stop are spaced along a dimension that said catheter shaft and said first shaft are collectively movable relative to each of said catheter handle and said proximal sleeve;
wherein said first stop defines at least in part one catheter shaft working length configuration and said second stop defines at least in part a different catheter shaft working length configuration.

91. The catheter of paragraph 90, wherein said catheter shaft comprises a third stop disposed within said proximal sleeve, wherein said catheter shaft comprises said third stop, wherein said first stop is engageable with said third stop to define said one catheter shaft working length configuration, wherein said second stop is engageable with said third stop to define said different catheter shaft working length configuration, and wherein said first stop and said second stop are each disposed in a fixed position relative to each of said actuator handle and said proximal sleeve.

92. The catheter of paragraph 91, wherein said actuator handle comprises said first stop and said proximal sleeve comprises said second stop, wherein an engagement of said third stop with said first stop defines a longer working catheter length than an engagement of said third stop with said second stop.

93. The catheter of paragraph 90, wherein said catheter shaft comprises said first stop and said second stop, wherein said catheter further comprises a third stop that is disposed in a fixed position relative to each of said actuator handle and said proximal sleeve, wherein said proximal sleeve comprises said third stop, wherein said first stop is engageable with one side of said third stop to define said one catheter shaft working length configuration, and wherein said second stop is engageable with an opposite side of said third stop to define said different catheter shaft working length configuration.

94. The catheter of paragraph 93, wherein said second stop is disposable beyond a proximal end of said proximal sleeve, and wherein said first stop remains disposed within said proximal sleeve.

95. The catheter of any of paragraphs 90-94, further comprising:
a catheter shaft clamp disposable in each of a clamping configuration and a released configuration relative to said catheter shaft; and
a clamping actuator operatively interconnected with said catheter shaft clamp to move said catheter shaft clamp between said clamping configuration and said released configuration.

96. The catheter of paragraph 95, wherein actuating said clamping actuator to dispose said catheter shaft clamp in said clamping configuration maintains said catheter shaft in a fixed position relative to each of said actuator handle and said proximal sleeve.

97. The catheter of any of paragraphs 90-96, wherein said actuator handle comprises first and second handle sections that are rotatable relative to one another, wherein said clamping actuator comprises said first and second handle section.

98. The catheter of any of paragraphs 1-3, further comprising:
a first handle;
a shaft interconnected with and movable in conjunction with said catheter shaft, wherein each of said catheter shaft and said shaft are movable relative to said first handle, wherein an outer perimeter of said shaft comprises first and second recesses that are spaced along a length dimension of said shaft; and
a detent movable relative to said shaft and separately disposable in each of said first and second recesses;
wherein said detent being in said first recess defines one catheter shaft working length configuration and said detent being in said second recess defines a different catheter shaft working length configuration.

99. The catheter of paragraph 98, wherein said detent is biased in a direction of said shaft.

100. The catheter of any of paragraphs 98-99, further comprising a detent actuator operatively interconnected with said detent to move said detent between at least two different positions relative to said shaft.

101. The catheter of paragraph 100, wherein actuation of said detent actuator moves said detent out of a respective one of said first recess and said second recess.

102. The catheter of any of paragraphs 100-101, wherein said detent actuator is located on an exterior of said catheter handle.

103. The catheter of paragraph 98, wherein a biasing member biases said detent to one of a locked position or an unlocked position relative to said shaft, and wherein said detent actuator disposes said detent in the other of said locked position or said unlocked position relative to said shaft.

104. The catheter of any of paragraphs 1-3, further comprising:
a first handle;
a shaft interconnected with and movable in conjunction with said catheter shaft, wherein each of said catheter shaft and said shaft are movable relative to said first handle, wherein an outer perimeter of said shaft comprises a shaft slot that extends along a length dimension of said shaft; and
a shaft clamp movable relative to said shaft and engageable with said shaft slot at a plurality of different positions along said slot;
wherein said shaft clamp engaging one location along said shaft slot defines one catheter shaft working length configuration and said shaft clamp engaging a different location along said shaft slot defines a different catheter shaft working length configuration.

105. The catheter of paragraph 104, wherein said shaft clamp is biased in a direction of said shaft slot.

106. The catheter of any of paragraphs 104-105 further comprising a clamp actuator operatively interconnected with said shaft clamp to move said shaft clamp between at least two different positions relative to said shaft.

107. The catheter of paragraph 106, wherein actuation of said clamp actuator moves said shaft clamp away from said shaft and into a released configuration relative to said shaft.

108. The catheter of any of paragraphs 106-107, wherein said clamp actuator is located on an exterior of said catheter handle.

109. The catheter of paragraph 104, wherein a biasing member biases said shaft clamp to one of a clamped position or an unclamped position relative to said shaft, and wherein said clamp actuator disposes said shaft clamp in the other of said clamped position or said unclamped position relative to said shaft.

110. The catheter of any of paragraphs 98-108, wherein said shaft is disposed within said first handle, wherein said shaft is axially movable relative to said first handle.

111. The catheter of any of paragraphs 1-3, wherein said catheter shaft actuator comprises an actuator handle and a catheter shaft clamp, wherein said catheter shaft clamp comprises a concave curved portion engageable with said catheter shaft.

112. The catheter of paragraph 111, wherein said actuator handle further comprises a release actuator operatively interconnected with said catheter shaft clamp.

113. The catheter of paragraph 112, wherein catheter shaft clamp is biased toward a clamping configuration relative to said catheter shaft, and wherein moving said release actuator relative to said actuator handle disposes said catheter shaft clamp in a released configuration relative to said catheter shaft.

114. The catheter of any of paragraphs 112-113, wherein said release actuator and said concave curved portion of said catheter shaft clamp are disposed on opposite sides of said catheter shaft.

115. The catheter of any of paragraphs 112-114, wherein said clamping actuator is biased toward a first position where said catheter shaft clamp is disposed in said clamping configuration, and wherein said clamping actuator is movable to a second position to dispose said catheter shaft clamp in said released configuration relative to said catheter shaft. 116. A catheter handle assembly, comprising:
a catheter handle comprising a distal end and a proximal end that are spaced along a first dimension; and
a shuttle connectable with a catheter shaft, wherein said shuttle is movable along said first dimension relative to at least said distal end of said catheter handle.

117. The catheter handle assembly of paragraph 116, wherein said catheter handle is a telescoping catheter handle.

118. The catheter handle assembly of paragraph 117, further comprising:
a first catheter handle configuration where said telescoping catheter handle is of a first handle length and said shuttle is spaced a first distance from said distal end of said telescoping catheter handle; and
a second catheter handle configuration where said telescoping catheter handle is of a second handle length and said shuttle is spaced a second distance from said distal end of said telescoping catheter handle;
wherein said first handle length is different from said second handle length and said first distance is different from said second distance.

119. The catheter handle assembly of paragraph 118, wherein said first handle length is less than said second handle length and said first distance is less than said second distance.

120. The catheter handle assembly of any of paragraphs 118-119, wherein each of said first catheter handle configuration and said second catheter handle configuration are pre-set.

121. The catheter handle assembly of any of paragraphs 118-120, wherein said telescoping catheter handle comprises first and second holes that are spaced along a length dimension of said telescoping catheter handle and that correspond with said first catheter handle configuration and said second catheter handle configuration, respectively, said catheter handle assembly further comprising a detent, wherein said detent is disposed in in said first hole when said telescoping catheter handle is in said first catheter handle configuration, and wherein said detent is disposed in said second hole when said telescoping catheter handle is in said second catheter handle configuration.

122. The catheter handle assembly of paragraph 121, wherein said detent is biased to a locking position.

123. The catheter handle assembly of any of paragraphs 117-122, further comprising a catheter shaft clamp disposable between a clamping and a released configuration all relative to a catheter shaft when interconnected with said shuttle.

124. The catheter handle assembly of paragraph 123, wherein said catheter shaft clamp is disposed on a distal end of said telescoping catheter handle.

125. The catheter handle assembly of any of paragraphs 117-124, wherein said telescoping catheter handle telescopes in a proximal direction.

126. The catheter handle assembly of any of paragraphs 117-125, wherein said shuttle is mounted to a proximal-most telescoping section of said telescoping catheter handle.

127. The catheter handle assembly of any of paragraphs 117-126, further comprising a generator connector on a proximal end of said telescoping catheter handle.

128. A catheter comprising a catheter shaft and the catheter handle assembly of any of paragraphs 117-127, wherein said catheter shaft extends distally from said shuttle.

129. The catheter of paragraph 128, wherein changing a position of said shuttle relative to said distal end of said telescoping catheter handle changes a working length of said catheter shaft.

130. The catheter handle assembly of paragraph 116, wherein said shuttle is movable along a length of said catheter handle between at least first and second positions that are spaced along said length of said catheter handle.

131. The catheter handle assembly of paragraph 130, wherein said shuttle is disposed within an interior of said catheter handle.

132. The catheter handle assembly of any of paragraphs 130-131, wherein said first and second positions are pre-set.

133. The catheter handle assembly of any of paragraphs 130-132, wherein said shuttle comprises a detent that is biased toward a perimeter wall of said catheter handle, wherein said perimeter wall comprises first and second apertures, wherein said detent protrudes through said first aperture and defines said first position when said detent is aligned with said first aperture, and wherein said detent protrudes through said second aperture and defines said second position when said detent is aligned with said second aperture.

134. The catheter handle assembly of any of paragraphs 130-132, wherein said catheter handle comprises a perimeter wall and a slot extending through said perimeter wall and extending between a first slot end and a second slot end, said catheter further comprising a shuttle shaft that extends from said shuttle through said slot, wherein said first slot end defines said first position and said second slot end defines said second position.

135. The catheter handle assembly of paragraph 134, further comprising a lock interconnected with said shuttle and disposed on an exterior of said catheter handle, wherein said lock is separately disposable in a locked configuration and an unlocked configuration, wherein said shuttle is movable relative to said catheter handle when said lock is in said unlocked configuration, and wherein a position of said shuttle is fixed relative to said catheter handle when said lock is in said locked configuration.

136. The catheter handle assembly of paragraph 135, wherein said lock is biased toward said locked configuration.

137. The catheter handle assembly of any of paragraphs 135-136, wherein said lock is movable away from said perimeter wall of said catheter shaft handle to dispose said lock in said unlocked configuration.

138. The catheter handle assembly of any of paragraphs 135-137, wherein said lock clamps against said catheter handle when said lock is disposed in said locked configuration.

139. The catheter handle assembly of any of paragraphs 135-138, wherein said lock comprises a knob.

140. The catheter handle assembly of any of paragraphs 130-132, further comprising:
a proximal shaft extending proximally from a proximal end of said catheter handle; and
a distal shaft extending distally from a distal end of said catheter handle;
wherein said shuttle is separately disposable in each of said proximal shaft and said distal shaft.

141. The catheter handle assembly of paragraph 140, wherein said proximal shaft and said distal shaft are of different lengths.

142. The catheter handle assembly of any of paragraphs 140-141, wherein said proximal shaft is longer than said distal shaft.

143. The catheter handle assembly of any of paragraphs 140-142, wherein said proximal shaft comprises a first aperture that extends through a perimeter wall of said proximal shaft and that corresponds with said first position, and wherein said distal shaft comprises a second aperture that extends through a perimeter wall of said distal shaft and that corresponds with said second position.

144. The catheter handle assembly of paragraph 143, wherein said shuttle comprises a detent that is biased toward said perimeter wall of said proximal shaft when said shuttle is disposed within said proximal shaft, wherein said detent is biased toward said perimeter wall of said distal shaft when said shuttle is disposed within said distal shaft, wherein said detent protrudes through said first aperture and defines said first position when said detent is aligned with said first aperture, and wherein said detent protrudes through said second aperture and defines said second position when said detent is aligned with said second aperture.

145. A catheter comprising a catheter shaft and the catheter handle assembly of any of paragraphs 130-144, wherein said catheter shaft extends distally from said catheter shaft mount of said shuttle.

146. The catheter of paragraph 145, wherein changing a position of said shuttle relative to said distal end of said telescoping catheter handle changes a working length of said catheter shaft.

147. The catheter handle assembly paragraph 116, further comprising:
a proximal sleeve extending proximally from a proximal end of said catheter handle; and
a first shaft that extends within said proximal sleeve;
a first stop; and
a second stop;
wherein said first stop and said second stop are spaced along a dimension that said shuttle is movable relative to each of said catheter handle and said proximal sleeve;
wherein said first stop defines at least in part one catheter shaft working length configuration and said second stop defines at least in part a different catheter shaft working length configuration.

148. The catheter handle assembly of paragraph 147, wherein said first stop is engageable with said shuttle to define said one catheter shaft working length configuration, wherein said second stop is engageable with said shuttle to define said different catheter shaft working length configuration, wherein said first stop and said second stop are each disposed in a fixed position relative to each of said catheter handle and said proximal sleeve.

149. The catheter handle assembly of paragraph 148, wherein said catheter handle comprises said first stop and said proximal sleeve comprises said second stop, wherein an engagement of said shuttle with said first stop defines a longer working catheter length than an engagement of said shuttle with said second stop.

150. The catheter handle assembly of paragraph 147, wherein said shuttle comprises a shaft, which in turn comprises said first stop and said second stop, wherein said catheter handle assembly further comprises a third stop that is disposed in a fixed position relative to each of said catheter handle and said proximal sleeve, wherein said first stop is engageable with one side of said third stop to define said one catheter shaft working length configuration, and wherein said second stop is engageable with an opposite side of said third stop to define said different catheter shaft working length configuration.

151. The catheter handle assembly of paragraph 150, wherein said proximal sleeve comprises said third stop, wherein said first stop is retained within said proximal sleeve, and wherein said second stop is disposed beyond a proximal end of said proximal sleeve.

152. The catheter handle assembly of any of paragraphs 147-151, further comprising:
a catheter shaft clamp disposable in each of a clamping configuration and a released configuration relative to said catheter shaft; and
a clamping actuator operatively interconnected with said catheter shaft clamp to move said catheter shaft clamp between said clamping configuration and said released configuration.

153. The catheter handle assembly of paragraph 152, wherein actuating said clamping actuator to dispose said catheter shaft clamp in said clamping configuration maintains said catheter shaft in a fixed position relative to each of said catheter handle and said proximal sleeve.

154. A catheter comprising a catheter shaft and the catheter handle assembly of any of paragraphs 147-153, wherein said catheter shaft extends through each of said catheter handle and said proximal sleeve, wherein a portion of said catheter shaft comprises said shuttle.

155. The catheter of paragraph 154, wherein changing a position of said shuttle relative to said distal end of said telescoping catheter handle changes a working length of said catheter shaft.

156. The catheter handle assembly of paragraph 116, wherein said shuttle comprises a shaft that is movable relative to said catheter handle, wherein an outer perimeter of said shaft comprises first and second recesses that are spaced along a length dimension of said shaft, said catheter handle assembly further comprising:
a detent movable relative to said shaft and separately disposable in each of said first and second recesses;
wherein said detent being in said first recess defines one catheter shaft working length configuration and said detent being in said second recess defines a different catheter shaft working length configuration.

157. The catheter handle assembly of paragraph 156, wherein said detent is biased in a direction of said shaft.

158. The catheter handle assembly of any of paragraphs 156-157, further comprising a detent actuator operatively interconnected with said detent to move said detent between at least two different positions relative to said shaft.

159. The catheter handle assembly of paragraph 158, wherein actuation of said detent actuator moves said detent out of a respective one of said first recess and said second recess.

160. The catheter handle assembly of any of paragraphs 158-159, wherein said detent actuator is located on an exterior of a handle.

161. The catheter handle assembly of paragraph 158, wherein a biasing member biases said detent to one of a locked position or an unlocked position relative to said shaft, and wherein said detent actuator disposes said detent in the other of said locked position or said unlocked position relative to said shaft.

162. The catheter of any of paragraphs 156-161, wherein said shaft is disposed within an interior of said catheter handle.

163. A catheter comprising a catheter shaft and the catheter handle assembly of any of paragraphs 156-162, wherein said catheter shaft extends distally from said shuttle.

164. The catheter of paragraph 163, wherein changing a position of said shuttle relative to said distal end of said catheter handle changes a working length of said catheter shaft.

165. The catheter handle assembly of paragraph 116, wherein said shuttle comprises a shaft, wherein said shaft is movable relative to said catheter handle, wherein an outer perimeter of said shaft comprises a shaft slot that extends along a length dimension of said shaft; and
a shaft clamp movable relative to said shaft and engageable with said shaft slot at a plurality of different positions along said slot;
wherein said shaft clamp engaging one location along said shaft slot defines one catheter shaft working length configuration and said shaft clamp engaging a different location along said shaft slot defines a different catheter shaft working length configuration.

166. The catheter handle assembly of paragraph 165, wherein said shaft clamp is biased in a direction of said shaft.

167. The catheter handle assembly of any of paragraphs 165-166, further comprising a clamp actuator operatively interconnected with said shaft clamp to move said shaft clamp between at least two different positions relative to said shaft.

168. The catheter handle assembly of paragraph 167, wherein actuation of said clamp actuator moves said shaft clamp away from said shaft and into a released configuration relative to said shaft.

169. The catheter handle assembly of any of paragraphs 167-168, wherein said clamp actuator is located on an exterior of a catheter handle.

170. The catheter handle assembly paragraphs 167-169, wherein a biasing member biases said shaft clamp to one of a clamped position or an unclamped position relative to said shaft, and wherein said clamp actuator disposes said shaft clamp in the other of said clamped position or said unclamped position relative to said shaft.

171. The catheter handle assembly of any of paragraphs 165-170, wherein said shaft is disposed within an interior of said catheter handle.

172. A catheter comprising a catheter shaft and the catheter handle assembly of any of paragraphs 165-171, wherein said catheter shaft extends distally from said shuttle.

173. The catheter of paragraph 172, wherein changing a position of said shuttle relative to said distal end of said telescoping catheter handle changes a working length of said catheter shaft.

174. A catheter, comprising:
a catheter shaft;
a catheter shaft actuator movably mounted on said catheter shaft;
a housing disposed in a fixed position relative to said catheter shaft, wherein at least part of said housing extends proximally relative to said catheter shaft actuator; and
a generator cable extending proximally from said housing.

175. The catheter of paragraph 174, further comprising:
a first configuration comprising a first distance between an actuator distal end of said catheter shaft actuator and a shaft distal end of said catheter shaft; and
a second configuration comprising a second distance between said actuator distal end and said shaft distal end, wherein said first distance and said second distance are different.

176. The catheter of any of paragraphs 174-175, wherein said catheter shaft actuator is configured to at least one of apply torque to said catheter shaft and advance said catheter shaft relative to a vasculature of a patient.

177. The catheter of any of paragraphs 174-176, further comprising a multi-function catheter shaft tool comprising:
a tool distal end;
a tool proximal end;
a lumen that extends from said tool distal end to said tool proximal end, wherein said lumen is sized to accommodate said catheter shaft;
a catheter shaft straightener comprising said tool distal end;
said catheter shaft actuator; and
a catheter shaft clamp disposable in each of a clamping configuration and a released configuration relative to said lumen;
wherein said catheter shaft straightener extends proximally from said tool distal end and at least to said actuator distal end, wherein said catheter shaft straightener and said catheter shaft actuator are configured to be collectively rotatable.

178. The catheter of paragraph 177, wherein said multi-function catheter shaft tool further comprises a clamping actuator operatively interconnected with said catheter shaft clamp.

179. The catheter of any of paragraphs 174-176, wherein said catheter shaft extends at least to a distal end of said housing, said catheter further comprising:
a catheter shaft clamp; and
a clamping actuator operatively connected with said catheter shaft clamp, wherein said clamping actuator is movable between at last two different positions to change said catheter shaft clamp between a released configuration relative to said catheter shaft and a clamping configuration relative to said catheter shaft;
wherein said catheter shaft actuator comprises said catheter shaft clamp and said clamping actuator, and wherein said catheter shaft actuator is movable along said catheter shaft and relative to each of said catheter shaft and said housing when said catheter shaft clamp is in said released configuration.

180. The catheter of any of paragraphs 174-176, wherein at least substantially an entirety of said catheter shaft actuator is disposed distally of said housing when said catheter shaft actuator is disposed in a proximal-most position relative to said catheter shaft.

181. The catheter of paragraph 180, wherein said catheter shaft actuator comprises a proximal housing section and a distal housing section, wherein a perimeter of said distal housing section is larger than said proximal housing section, and wherein said distal housing section comprises said clamping actuator and said catheter shaft clamp.

182. The catheter of any of paragraphs 174-176, wherein said catheter shaft actuator comprises an actuator handle.

183. The catheter of paragraph 182, wherein a body of said catheter shaft actuator comprises at least one curved surface proceeding along a length dimension of said body.

184. The catheter of any of paragraphs 174-176, wherein a proximal end section of said catheter shaft actuator is disposed within a distal end section of said housing when said catheter shaft actuator is disposed in a proximal-most position relative to said catheter shaft.

185. The catheter of paragraph 184, wherein said catheter shaft actuator comprises a proximal housing section and a distal housing section, wherein a perimeter of said distal housing section is larger than said proximal housing section, and wherein said distal housing section comprises a clamping actuator and a catheter shaft clamp.

186. The catheter of paragraph 185, wherein at least substantially an entirety of said proximal housing section of said catheter shaft actuator is disposed within said distal end section of said housing when said catheter shaft actuator is disposed in a proximal-most position relative to said catheter shaft.

187. The catheter of any of paragraphs 174-176, wherein a majority of said housing is disposed within a proximal end section of said catheter shaft actuator when said catheter shaft actuator is disposed in a proximal-most position relative to said catheter shaft.

188. The catheter of paragraph 187, wherein said catheter shaft actuator comprises a handle.

189. The catheter of any of paragraphs 174-176, wherein a distal end section of said housing is disposed within a proximal end section of said catheter shaft actuator when said catheter shaft actuator is disposed in a proximal-most position relative to said catheter shaft.

190. The catheter of paragraph 189, wherein a majority of said housing is disposed proximally of said catheter shaft actuator when said catheter shaft actuator is disposed in a proximal-most position relative to said catheter shaft.

191. The catheter of any of paragraphs 189-190, wherein a body of said catheter shaft actuator comprises at least one curved surface proceeding along a length dimension of said body.

192. The catheter of any of paragraphs 174-176, further comprising a catheter handle, wherein said catheter shaft extends at least to a distal end of said catheter handle, wherein at least part of said catheter shaft actuator is disposed distally of said catheter handle, and wherein said catheter shaft actuator comprises:
a passage extending through said catheter shaft actuator from said actuator distal end to an actuator proximal end, wherein said catheter shaft extends entirely through said catheter shaft actuator;
a clamping actuator on an exterior of said catheter shaft actuator;
a catheter shaft clamp operatively interconnected with said clamping actuator;
a clamping configuration, relative to said catheter shaft, when said clamping actuator is in a first position; and
a released configuration, relative to said catheter shaft, when said clamping actuator is in a second position, wherein said clamping actuator is movable between said first position and said second position to dispose said clamp in said clamping configuration and said released configuration, respectively.

193. The catheter of paragraph 192, wherein said catheter shaft actuator is movable along said catheter shaft and relative to each of said catheter shaft and said catheter handle when said clamping actuator is in said first position.

194. The catheter of any of paragraphs 192-193, wherein said catheter shaft actuator further comprises:
a body;
a first tube extending distally from said body, wherein said catheter shaft extends through said first tube;
a second tube extending proximally from said body, wherein said first tube and said second tube are spaced along a length of said catheter shaft actuator, and wherein said passage comprises an interior of said first tube and an interior of said second tube.

195. The catheter of paragraph 194, wherein said second tube extends into a distal end of said catheter handle when said catheter is disposed in one catheter shaft working length configuration.

196. The catheter of any of paragraphs 194-195, wherein said second tube is spaced from an entirety of said catheter handle when said catheter is disposed in another catheter shaft working length configuration.

197. The catheter of any of paragraphs 194-196, wherein said catheter shaft clamp is located in a space between said first tube and said second tube within said body.

198. The catheter of any of paragraphs 194-197, wherein said clamping actuator is disposed on an exterior of said body.

199. The catheter of any of paragraphs 192-193, wherein said catheter shaft actuator further comprises a body, wherein said body extends into a distal end of said catheter handle when said catheter is disposed in one catheter shaft working length configuration.

200. The catheter of paragraph 199, wherein said catheter shaft actuator is movable along said catheter shaft and relative to each of said catheter shaft and said catheter handle when said clamping actuator is in said first position.

201. The catheter of any of paragraphs 199-200, wherein said body of said catheter shaft actuator comprises a proximal housing section and a distal housing section, wherein a perimeter of said distal housing section is larger than said proximal housing section, wherein said distal housing section comprises said clamping actuator and said catheter shaft clamp, and wherein said proximal housing section is disposed within said catheter shaft handle when said catheter is disposed in said one catheter shaft working length configuration.

202. The catheter of any of paragraphs 199-201, wherein an entirety of said catheter shaft actuator is spaced from said catheter handle, along said catheter shaft, when said catheter is disposed in another catheter shaft working length configuration.

203. The catheter of any of paragraphs 174-176, wherein said catheter shaft actuator comprises an actuator handle and a clamping actuator connectable with said actuator handle, wherein said actuator handle comprises an actuation surface, wherein said clamping actuator comprises a clamp, wherein said clamp extends into said actuator handle and interfaces with said actuation surface, and wherein said catheter shaft extends through said clamp of said clamping actuator and through said actuator handle.

204. The catheter of paragraph 203, wherein said clamping actuator is threadably connected to said actuator handle.

205. The catheter of any of paragraphs 203-204, wherein said clamping actuator is disposed on a distal end section of said actuator handle.

206. The catheter of any of paragraphs 203-205, wherein axially moving said clamping actuator relative said actuator handle causes said actuation surface of said actuator handle to compress said clamp of said clamping actuator into clamping engagement with said catheter shaft.

207. The catheter of paragraph 206, wherein said clamping actuator is rotatably connected with said actuator handle, and wherein rotating said clamping actuator relative to said actuator handle also moves said clamping actuator axially relative to said actuator handle.

208. The catheter of any of paragraphs 203-207, wherein said catheter shaft extends at least to a distal end of said housing, wherein at least a distal end section of said housing is disposed within a proximal end section of said actuator handle when said catheter shaft actuator is disposed in a proximal-most position relative to said catheter shaft.

209. The catheter of paragraph 208, wherein a majority of said housing is disposed within said actuator handle when said catheter shaft actuator is disposed in a proximal-most position relative to said catheter shaft.

210. The catheter of any of paragraphs 174-176, wherein said catheter shaft actuator comprises an actuator handle and a clamping actuator connectable with said actuator handle, wherein said actuator handle comprises a clamp, wherein said clamping actuator comprises an actuation surface, wherein said clamp of said actuator handle extends into said clamping actuator and interfaces with said actuation surface, and wherein said catheter shaft extends through said clamping actuator and through said clamp of said actuator handle.

211. The catheter of paragraph 210, wherein said clamping actuator is threadably connected to said handle.

212. The catheter of any of paragraphs 210-211, wherein said clamping actuator is disposed on a distal end section of said actuator handle.

213. The catheter of any of paragraphs 210-212, wherein axially moving said clamping actuator relative said actuator handle causes said actuation surface of said clamping actuator to compress said clamp of said actuator handle into clamping engagement with said catheter shaft.

214. The catheter of paragraph 213, wherein said clamping actuator is rotatably connected with said actuator handle, and wherein rotating said clamping actuator relative to said actuator handle also moves said clamping actuator axially relative to said actuator handle.

215. The catheter of any of paragraphs 174-176, wherein said catheter shaft actuator comprises an actuator handle, wherein said actuator handle comprises first and second clamping segments that are separated by a space, wherein said catheter shaft extends through said space between said first and second clamping segments, and wherein said actuator handle further comprises a clamping actuator engageable with said first and second clamping segments.

216. The catheter of paragraph 215, wherein said first and second clamping segments are biased toward a clamping configuration relative to said catheter shaft, and wherein moving said clamping actuator relative to said first and second clamping segments disposes said first and second clamping segments in a released configuration relative to said catheter shaft.

217. The catheter of paragraph 216, wherein said clamping actuator is movable between said first and second positions, wherein first and second clamping segments are in said clamping configuration when said clamping actuator is in said first position and where said first and second clamping segments are separated by a first space, and wherein said first and second clamping segments are in said released configuration when said clamping actuator is in said second position and where said first and second clamping segments are separated by a second space to allow said catheter shaft actuator to move along and relative to said catheter shaft, wherein said second space is larger than said first space.

218. The catheter of paragraph 217, wherein said clamping actuator is biased toward said first position.

219. The catheter of any of paragraphs 174-176, wherein said catheter shaft actuator comprises an actuator handle, a clamping actuator, and a rotary catheter shaft clamp comprising a first clamping segment and a second clamping segment.

220. The catheter of paragraph 219, wherein said first clamping segment and said second clamping segment are engageable with opposite sides of said catheter shaft.

221. The catheter of any of paragraphs 219-220, further comprising a first biasing member engaged with said first clamping segment on a first side of an axis about which said rotary catheter shaft clamp is rotatable, and wherein said clamping actuator is engageable with said second clamping segment on an opposite second side of said axis.

222. The catheter of paragraph 221, wherein said first biasing member simultaneously biases said first clamping segment and said second clamping segment into a clamped configuration for said catheter shaft clamp relative to said catheter shaft, and wherein moving said clamping actuator relative to said first clamping segment disposes said catheter shaft clamp in a released configuration relative to said catheter shaft to allow said catheter shaft actuator to move along and relative to said catheter shaft.

223. The catheter of paragraph 222, wherein said clamping actuator is biased to a position where said catheter shaft clamp is disposed in said clamped configuration relative to said catheter shaft.

224. The catheter of any of paragraphs 219-223, wherein said catheter shaft extends at least to a distal end of said housing, wherein at least a distal end section of said housing is disposed within a proximal end section of said actuator handle when said catheter shaft actuator is disposed in a proximal-most position relative to said catheter shaft.

225. The catheter of paragraph 224, wherein a majority of said housing is disposed proximally beyond said actuator handle when said catheter shaft actuator is disposed in a proximal-most position relative to said catheter shaft.

226. The catheter of any of paragraphs 174-176, wherein said catheter shaft actuator comprises an actuator handle and a clamping actuator, wherein said clamping actuator comprises a collet rotatably connected with an outer perimeter of said actuator handle, wherein said catheter shaft extends through each of said actuator handle and said clamping actuator, wherein said collet is rotatable between at least first and second positions relative to said actuator handle, wherein said actuator handle is disposed in a clamping configuration, relative to said catheter shaft, when said collet is in said first position, and wherein said actuator handle is disposed in a released configuration, relative to said catheter shaft, when said collet is in said second position.

227. The catheter of any of paragraphs 174-176, wherein said catheter shaft actuator comprises an actuator handle, a first clamp, and a clamping actuator, wherein said catheter shaft extends through said actuator handle, wherein said first clamp is movable along a first axis relative to said catheter shaft between a clamping configuration and a released configuration, wherein said clamping actuator is movable along a second axis between at least first and second positions to dispose said first clamp in said clamping configuration and said released configuration, respectively, wherein said first clamp clamps against said catheter shaft in said clamping configuration, and wherein said catheter shaft actuator is movable along and relative to said catheter shaft when said first clamp is in said released configuration.

228. The catheter of paragraph 227, wherein said second axis is orthogonal to said catheter shaft.

229. The catheter of any of paragraphs 227-228, wherein said first axis is parallel to said catheter shaft.

230. The catheter of any of paragraphs 227-229, further comprising a biasing member disposed between said clamping actuator and said first clamp when said clamping actuator is in said first position.

231. The catheter of any of paragraphs 174-176, wherein said catheter shaft actuator comprises an actuator handle, a first clamp, a clamping actuator interconnected with said first clamp, and a biasing member that engages at least one of said first clamp and said clamping actuator, wherein said catheter shaft extends through said actuator handle, wherein said clamping actuator is movable between at least first and second positions and said first clamp is movable between a clamping configuration and a released configuration, relative to said catheter shaft, wherein one of said clamping actuator and said biasing member disposes said first clamp in said clamping configuration, and wherein the other of said clamping actuator and said biasing member disposes said first clamp in said released configuration.

232. The catheter of paragraph 231, wherein said first clamp moves in a first direction relative to said actuator handle in moving between said clamping configuration and said released configuration.

233. The catheter of paragraph 232, wherein said first direction is orthogonal to said a length dimension of catheter shaft.

234. The catheter of any of paragraphs 231-233, wherein said biasing member disposes said first clamp in said clamping configuration when said clamping actuator is in said first position, and wherein moving said clamping actuator from said first position to said second position compresses said biasing member and disposes said first clamp in said released configuration.

235. The catheter of any of paragraphs 174-176, wherein said catheter shaft actuator comprises an actuator handle and a catheter shaft clamp, wherein said catheter shaft clamp comprises a concave curved portion engageable with said catheter shaft.

236. The catheter of paragraph 235, wherein said actuator handle further comprises a release actuator operatively interconnected with said catheter shaft clamp.

237. The catheter of paragraph 236, wherein catheter shaft clamp is biased toward a clamping configuration relative to said catheter shaft, and wherein moving said release actuator relative to said actuator handle disposes said catheter shaft clamp in a released configuration relative to said catheter shaft.

238. The catheter of any of paragraphs 236-237, wherein said release actuator and said concave curved portion of said catheter shaft clamp are disposed on opposite sides of said catheter shaft.

239. The catheter of any of paragraphs 236-238, wherein said clamping actuator is biased toward a first position where said catheter shaft clamp is disposed in said clamping configuration, and wherein said clamping actuator is movable to a second position to dispose said catheter shaft clamp in said released configuration relative to said catheter shaft.

240. A method of operating a catheter, said catheter comprising a catheter shaft actuator and a catheter shaft, said method comprising the steps of:
introducing a distal end of said catheter shaft into a vasculature access;
executing a first moving step comprising moving said catheter shaft actuator relative to each of said catheter shaft and said vasculature access to reduce a spacing between said catheter shaft actuator and said vasculature access and to dispose said catheter shaft actuator into a first relative position;
fixing said catheter shaft actuator relative to said catheter shaft in said first relative position;
executing a first advancing step comprising advancing each of said catheter shaft actuator and said catheter shaft relative to said vasculature access to reduce a spacing between said catheter shaft actuator and said vasculature, wherein said first advancing step is executed after said fixing step for said catheter shaft actuator being in said first relative position;
executing a second moving step comprising moving said catheter shaft actuator relative to each of said catheter shaft and said vasculature access to increase a spacing between said catheter shaft actuator and said vasculature access and to dispose said catheter shaft actuator into a second relative position;
fixing said catheter shaft actuator relative to said catheter shaft in said second relative position; and
executing a second advancing step comprising advancing each of said catheter shaft actuator and said catheter shaft relative to said vasculature access to reduce a spacing between said catheter shaft actuator and said vasculature, wherein said second advancing step is executed after said fixing step for said catheter shaft actuator being in said second relative position.

### BRIEF DESCRIPTION OF THE DRAWINGS

FIG. 1A is a schematic of a neuromodulation/denervation system.

FIG. 1B is a schematic of introducing a catheter shaft of the neuromodulation/denervation system of FIG. 1A into a patient's vasculature through a femoral access.

FIG. 2 is a schematic of a vasculature of a patient, illustrating both a typical femoral access and a radial access.

FIG. 3A is a perspective view of a prior art neuromodulation/denervation catheter with a standalone catheter shaft straightener.

FIG. 3B is an exploded perspective view of a portion of the neuromodulation/denervation catheter of FIG. 3A.

FIG. 3C is a perspective view of a distal end section of the catheter shaft for the neuromodulation/denervation catheter of FIG. 3A.

FIG. 3D is a side view of one of the electrodes and associated wiring from the distal end section of the catheter shaft for the neuromodulation/denervation catheter of FIG. 3A.

FIG. 3E is a cross-sectional schematic showing a lumen through the distal end section of the catheter shaft for the neuromodulation/denervation catheter of FIG. 3A, and through which a guidewire may be directed.

FIG. 3F is a plan view of a printed circuit board used by the neuromodulation/denervation catheter of FIG. 3A.

FIG. 4A is a perspective view of the neuromodulation/denervation catheter of FIGS. 3A-3F, with its catheter shaft being adapted for femoral access to the patient's vasculature.

FIG. 4B is a perspective view of the neuromodulation/denervation catheter of FIGS. 3A-3F, with its catheter shaft being adapted for radial access to the patient's vasculature.

FIG. 5A is a side view of an embodiment of a multi-function catheter shaft tool.

FIG. 5B illustrates a lumen that extends through the multi-function catheter shaft tool of FIG. 5A.

FIG. 6A is a side view of another embodiment of part of a multi-function catheter shaft tool.

FIG. 6B is a perspective view of a proximal end section of the part of the multi-function catheter shaft tool shown in FIG. 6A.

FIG. 6C is a cutaway, side view of the part of the multi-function catheter shaft tool shown in FIG. 6A.

FIG. 6D is a perspective side view of a cap that is used in combination with the structure of FIGS. 6A-6C to define a multi-function catheter shaft tool.

FIG. 6E is a transparent perspective side view of the cap shown in FIG. 6D.

FIG. 7A is a transparent side view of the assembled components shown in FIG. 6A-6E to define the multi-function catheter shaft tool.

FIG. 7B is another transparent side view of the assembled components shown in FIG. 6A-6E to define the multi-function catheter shaft tool.

FIG. 7C is a side view of the assembled components shown in FIG. 6A-6E to define the multi-function catheter shaft tool.

FIG. 8A is a perspective view of an embodiment of a catheter assembly that utilizes both a catheter handle and a separate catheter shaft actuator, and with the catheter shaft actuator being in a first position relative to the catheter handle.

FIG. 8B is a perspective view of the catheter assembly of FIG. 8A, with the catheter shaft actuator being in a second position relative to the catheter handle.

FIG. 8C is an enlarged, perspective view of a section of the catheter shaft actuator for the catheter assembly of FIG. 8A.

FIG. 8D is an enlarged, cutaway view of the catheter shaft actuator for the catheter assembly of FIG. 8A.

FIG. 9A is a perspective view of an embodiment of a catheter assembly that utilizes both an electrical housing and a separate catheter shaft actuator.

FIG. 9B is a perspective view of the catheter shaft actuator for the catheter assembly of FIG. 9A.

FIG. 9C is an exploded, perspective view of the catheter shaft actuator for the catheter assembly of FIG. 9A.

FIG. 9D is an exploded, perspective view of the electrical housing for the catheter assembly of FIG. 9A.

FIG. 10A is a perspective view of another embodiment of a catheter assembly that utilizes both a catheter handle and a separate catheter shaft actuator, and with the catheter shaft actuator in a first position relative to the catheter handle.

FIG. 10B is a cutaway view of the catheter assembly of FIG. 10A, with the catheter shaft actuator being in a second position relative to the catheter handle.

FIGS. 11A1-11A4 are views of a catheter assembly that includes an actuator handle, a housing, and a catheter shaft, where the housing protrudes proximally beyond the actuator handle in one configuration.

FIGS. 11B1-11B3 are views of a catheter assembly that includes an actuator handle, a housing, and a catheter shaft, where a substantial portion of the housing is disposable within an interior of the actuator handle in one configuration.

FIGS. 11B4-11B5 are views of a catheter assembly that includes an actuator handle, a housing, and a catheter shaft, where the housing protrudes proximally beyond the actuator handle in one configuration.

FIGS. 12A1-12A4 are views of an actuator handle having a catheter shaft clamp, along with clamping actuator that is rotatably connectable with the actuator handle.

FIGS. 12B1-12B3 are views of an actuator handle having a clamping actuator, along with a catheter shaft clamp that is rotatably connectable with the actuator handle.

FIGS. 12C1-12C4 are views of an actuator handle having a catheter shaft clamp, along with an axially-movable clamping actuator.

FIGS. 12D1-12D5 are views of an actuator handle having a rotary catheter shaft clamp.

FIG. 13A is a schematic of an actuator handle having a catheter shaft clamp, along with a collet that is rotatably connected with the actuator handle to dispose the catheter shaft clamp in a clamped configuration.

FIG. 13B is a schematic of an actuator handle having a catheter shaft clamp, where a clamping actuator moves in one dimension to move the catheter shaft clamp in a different dimension.

FIG. 13C is a schematic of an actuator handle having a clamping actuator that includes both an actuation section and a clamping section.

FIGS. 14A-14D are views of an actuator handle that utilizes a telescoping configuration.

FIGS. 15A-15G are views of an actuator handle in which a shuttle is movably disposed, is connectable to a catheter shaft, and is disposable in a plurality of fixed positions along the actuator handle.

FIGS. 16A-16H are views of an actuator handle having a slot that extends through the actuator handle and through which a knob extends to interconnect with a shuttle that is movably disposed with the actuator handle and that is connectable to a catheter shaft.

FIGS. 17A-17I are views of an actuator handle having a primary handle section, a distal handle section, and a proximal handle section, with a shuttle being movably disposed within the actuator handle, connectable to a catheter shaft, and disposable in at least one fixed position in each of the distal handle section and the proximal handle section.

FIGS. 18A1-18A6 are views of an actuator handle having a proximal sleeve in which a movable stop is disposed and that establishes a range of motion of a catheter shaft relative to the actuator handle.

FIGS. 18B1-B3 are views of an actuator handle with a proximal sleeve in which one movable stop is disposed within the proximal sleeve and another movable stop is disposed outside of the proximal sleeve, and that collectively establish a range of motion of a catheter shaft relative to the actuator handle.

FIG. 18C1 is a schematic of a shaft that may be used with an actuator handle, and that includes a plurality of recesses spaced along its length to establish different positions of the shaft relative to the actuator handle.

FIGS. 18C2-C3 are schematics of different clamping arrangements that may be used by an actuator handle when incorporating the shaft shown in FIG. 18C1.

FIG. 18D1 is a schematic of a shaft that may be used with an actuator handle, and that includes an elongate recess that accommodates different positions of the shaft relative to the actuator handle.

FIGS. 18D2-18D3 are schematics of different clamping arrangements that may be used by an actuator handle when incorporating the shaft shown in FIG. 18D1.

FIG. 19 is schematic of a clamping arrangement that may be used with a shaft that may be used with an actuator handle, where the clamping arrangement may be used to fix the shaft relative to the actuator handle at various locations along the length of the shaft.

FIG. 20 is a view of a catheter that may be adapted to use any of the configures of FIGS. 18C1 through FIG. 19.

### DETAILED DESCRIPTION

The present disclosure is applicable to apparatuses and methods for achieving electrically and/or thermally-induced neuromodulation/denervation (e.g., rendering neural fibers that innervate the kidney inert, inactive or otherwise completely or partially reduced in function) by percutaneous transluminal intravascular access. Embodiments of the present technology relate to a treatment device (e.g., treatment catheter) having a therapeutic assembly (e.g., one or more energy elements) on a distal portion of the catheter shaft. After deployment in a target blood vessel of a human patient, the distal portion of the assembly is transformable between a delivery state having a low-profile that is configured to pass through the vasculature and a deployed state having a radially expanded shape (e.g., generally spiral/helical or coil) in which the distal portion is maintained in stable apposition with an inner wall of the target blood vessel (e.g., renal artery).

The system can also include an energy source or energy generator external to the patient in electrical communication with energy elements of the therapeutic assembly. In operation, the energy elements are advanced to a target blood vessel, such as the renal artery, along a percutaneous transluminal path (e.g., a femoral artery puncture, an iliac artery and the aorta, a radial artery, or another suitable intravascular path), and then energy is delivered to the wall of the target blood vessel via the energy elements. Suitable energy modalities include, for example, electrical energy, radio frequency (RF) energy, pulsed electrical energy, ultrasound, or thermal energy. The treatment device carrying the energy elements can be configured such that the energy elements are in constant apposition with the interior wall of the target blood vessel when in the deployed state (e.g., radially expanded to have a spiral/helical shape). The pre-formed spiral/helical shape of the deployed portion allows blood to flow through the assembly during therapy, which is expected to help cool the therapy assembly to prevent clot formation that may result in occlusion of the blood vessel during activation of the energy elements. The spiral/helical shape also enhances the apposition of the energy elements with the inner wall of target blood vessels and makes the therapeutic assembly adaptable to a range of vessel diameters. The largest diameter vessel in the range is at least slightly smaller than the free or un-constrained diameter of the pre-formed spiral/helical shape in order to provide and maintain adequate contact between the energy elements and the vessel wall.

Specific details of several embodiments are described herein. Although many of the embodiments are described with respect to devices, systems, and methods for intravascular renal neuromodulation, other applications and other embodiments in addition to those described herein are within the scope of the present technology. For example, at least some embodiments of the present technology may be useful for intraluminal neuromodulation, extravascular neuromodulation, non-renal neuromodulation, and/or use in therapies other than neuromodulation. It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

As used herein, the terms “distal” and “proximal” define a position or direction with respect to a clinician or a clinician's control device (e.g., a handle of a neuromodulation catheter). The terms, “distal”, “distally”, or the like refer to a position distant from or in a direction away from a clinician or a clinician's control device along the length of device. The terms “proximal”, “proximally”, or the like refer to a position near or in a direction toward a clinician or a clinician's control device along the length of device.

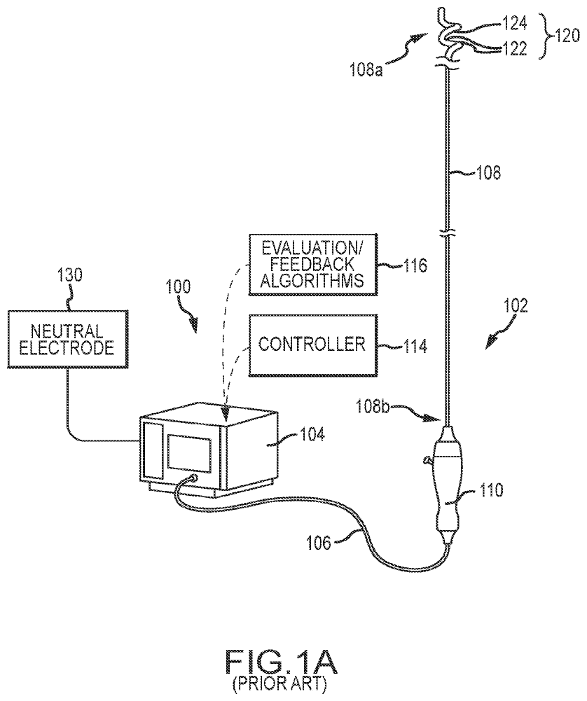

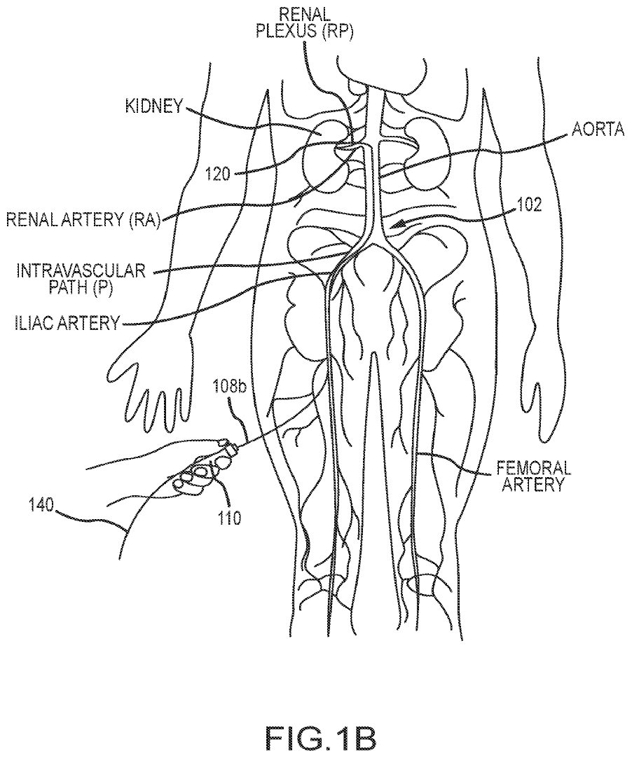

FIG. 1A is a schematic of a neuromodulation/denervation system 100. The system 100 100 includes a neuromodulation/denervation catheter 102, a console 104, and a cable 106 extending therebetween. Cable 106 may provide a permanent connection between catheter 102 and console 104, or cable 106 may be disconnectable (e.g. to permit the use of console 104 with different catheters). The neuromodulation/denervation catheter 102 can include an elongated shaft 108 having a proximal portion 108b, a distal portion 108a, a handle 110 operably connected to the shaft 108 at the proximal portion 108b, and a neuromodulation/denervation assembly 120 operably connected to and/or comprising at least a part of the distal portion 108a. The diameter of shaft 108 and the neuromodulation/denervation assembly 120 can be 2, 3, 4, 5, 6, or 7 French or another suitable size. The neuromodulation/denervation assembly 120 can include two or more metallic elements 122 that extend longitudinally along at least a portion of the length of the neuromodulation/denervation assembly 120 and a dielectric material 124 between the metallic elements 122. The metallic elements 122 can be elongated electrodes that extend longitudinally along the neuromodulation/denervation assembly 120 and are configured to apply electrical stimuli (e.g., RF energy) to target sites at or proximate to vessels within a patient, to temporarily stun nerves, to deliver neuromodulation energy to target sites, and/or to detect vessel impedance. In various embodiments, certain metallic elements 122 can be dedicated to applying stimuli and/or detecting impedance, and the neuromodulation/denervation assembly 120 can include other types of therapeutic elements that provide neuromodulation therapy using various modalities, such cryotherapeutic cooling, ultrasound energy, etc. The dielectric material 124 can be an elongated element that extends along at least a portion of the length of the neuromodulation/denervation assembly 120 and separates the metallic elements 122 from each other along at least a portion of the length of the metallic elements 122.

The distal portion 108a of the shaft 108 is configured to be moved within a lumen of a human patient and locate the neuromodulation/denervation assembly 120 at a target site within or otherwise proximate to the lumen. For example, shaft 108 can be configured to position the neuromodulation/denervation assembly 120 within a blood vessel, a duct, an airway, or another naturally occurring lumen within the human body. In certain embodiments, intravascular delivery of the neuromodulation/denervation assembly 120 includes percutaneously inserting a guide wire 140 (FIG. 1B) into a body lumen of a patient and moving the shaft 108 and/or the neuromodulation/denervation assembly 120 along the guide wire until the neuromodulation/denervation assembly 120 reaches a target site (e.g., a renal artery). For example, the distal end of the neuromodulation/denervation assembly 120 or other part of the distal portion 108a of the shaft 108 may include a passageway for engaging (e.g., receiving) the guide wire for delivery of the neuromodulation/denervation assembly 120 using over-the-wire (OTW) or rapid exchange (RX) techniques. In other embodiments, the neuromodulation/denervation catheter 102 can be a steerable or non-steerable device configured for use without a guide wire. In still other embodiments, the neuromodulation/denervation catheter 102 can be configured for delivery via a guide catheter or sheath.

Once at the target site, the neuromodulation/denervation assembly 120 can be configured to apply stimuli, detect resultant hemodynamic responses, and provide or facilitate neuromodulation therapy at the target site (e.g., using the metallic elements 122 and/or other energy delivery elements). For example, the neuromodulation/denervation assembly 120 can detect vessel impedance via the metallic elements 122, detect blood flow via a flow sensing element (e.g., a Doppler velocity sensing element (not shown)), detect local blood pressure within the vessel via a pressure transducer or other pressure sensing element (not shown), and/or detect other hemodynamic parameters. The detected hemodynamic responses can be transmitted to the console 104 and/or another device external to the patient. The console 104 can be configured to receive and store the recorded hemodynamic responses for further use by a clinician or operator. For example, a clinician can use the hemodynamic responses received by the console 104 to determine whether an application of neuromodulation energy was effective in modulating nerves to a desired degree.

The console 104 can be configured to control, monitor, supply, and/or otherwise support operation of the neuromodulation/denervation catheter 102. The console 104 can further be configured to generate a selected form and/or magnitude of energy for delivery to tissue at the target site via the neuromodulation/denervation assembly 120, and therefore the console 104 may have different configurations depending on the treatment modality of the neuromodulation/denervation catheter 102. For example, when the neuromodulation/denervation catheter 102 is configured for electrode-based, heat-element-based, or transducer-based treatment, the console 104 can include an energy generator (not shown) configured to generate RF energy (e.g., monopolar and/or bipolar RF energy), pulsed electrical energy, microwave energy, optical energy, ultrasound energy (e.g., intravascularly delivered ultrasound, and/or HIFU), direct heat energy, radiation (e.g., infrared, visible, and/or gamma radiation), and/or another suitable type of energy. When the neuromodulation/denervation catheter 102 is configured for cryotherapeutic treatment, the console 104 can include a refrigerant reservoir (not shown), and can be configured to supply the neuromodulation/denervation catheter 102 with refrigerant. Similarly, when the neuromodulation/denervation catheter 102 is configured for chemical-based treatment (e.g., drug infusion), the console 104 can include a chemical reservoir (not shown) and can be configured to supply the neuromodulation/denervation catheter 102 with one or more chemicals. In some embodiments, the console 104 can include one or more fluid reservoirs (not shown) for coolant and/or irrigant (e.g., saline) to be delivered to the metallic elements 122 and/or the dielectric material 124.

In selected embodiments, the system 100 may be configured to deliver a monopolar electric field via one or more of the metallic elements 122. In such embodiments, a neutral or dispersive electrode 130 may be electrically connected to the console 104 and attached to the exterior of the patient. In embodiments including multiple metallic elements 122, the metallic elements 122 may deliver power independently (i.e., may be used in a monopolar fashion), either simultaneously, selectively, or sequentially, and/or may deliver power between any desired combination of the metallic elements 122 (i.e., may be used in a bipolar fashion). In addition, an operator optionally may be permitted to choose which metallic elements 122 are used for power delivery in order to form customized lesion(s) within the renal artery, as desired. One or more sensing elements (not shown), such as one or more temperature (e.g., thermocouple, thermistor, etc.), pressure, optical, flow, chemical, and/or other sensing elements, may be located proximate to, within, or integral with the metallic elements 122. The sensing element(s) and the metallic elements 122 can be connected to one or more supply wires (not shown) that transmit signals from the sensing element(s) and/or convey energy to the metallic elements 122.

In various embodiments, the system 100 can further include a controller 114 communicatively coupled to the neuromodulation/denervation catheter 102. The controller 114 can be configured to initiate, terminate, and/or adjust operation of one or more components (e.g., the metallic elements 122) of the neuromodulation/denervation catheter 102 directly and/or via the console 104. In other embodiments, the controller 114 can be omitted or have other suitable locations (e.g., within the handle 110, along the cable 106, etc.). The controller 114 can be configured to execute an automated control algorithm and/or to receive control instructions from an operator. Further, the console 104 can be configured to provide feedback to an operator before, during, and/or after a treatment procedure via an evaluation/feedback algorithm 116.

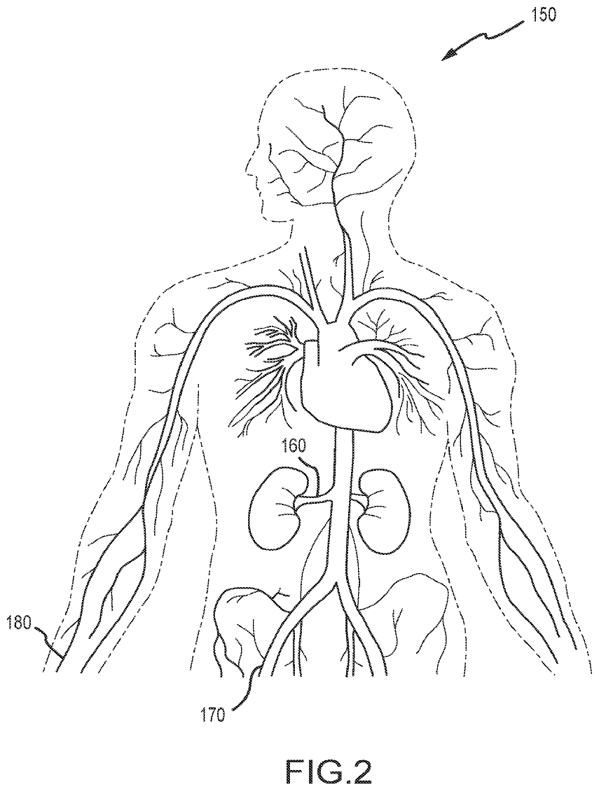

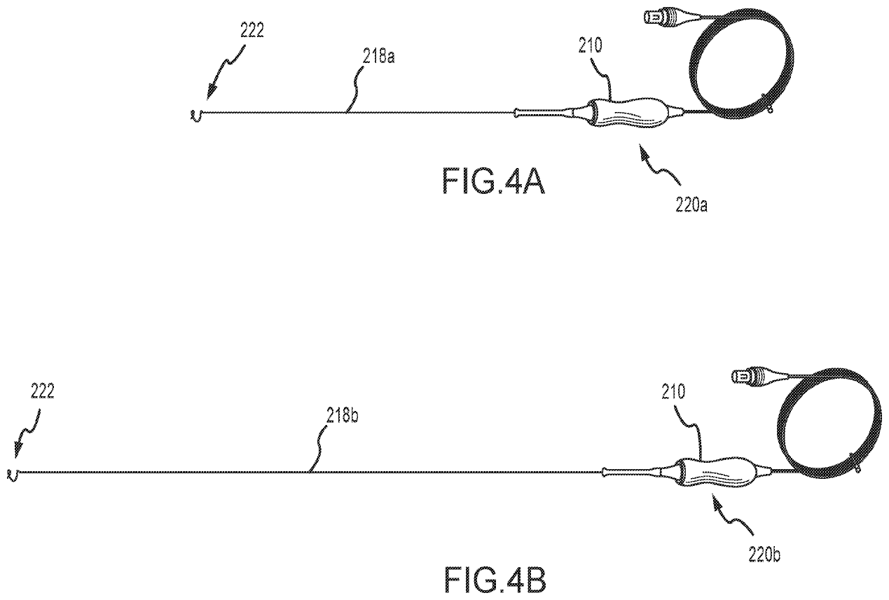

A schematic of a vasculature for a patient 150 is presented in FIG. 2. The renal artery 160, a representative femoral access 170, and a representative radial access 180 are shown in relation to this vasculature. A catheter (e.g., catheter 220a shown in FIG. 4A and addressed below) may be introduced through the femoral access 170 and may be directed through the patient's vasculature to reach a renal artery 160. A catheter (e.g., catheter 220b shown in FIG. 4B and addressed below) may also be introduced through the radial access 180 and may be directed through the patient's vasculature to reach a renal artery 160.

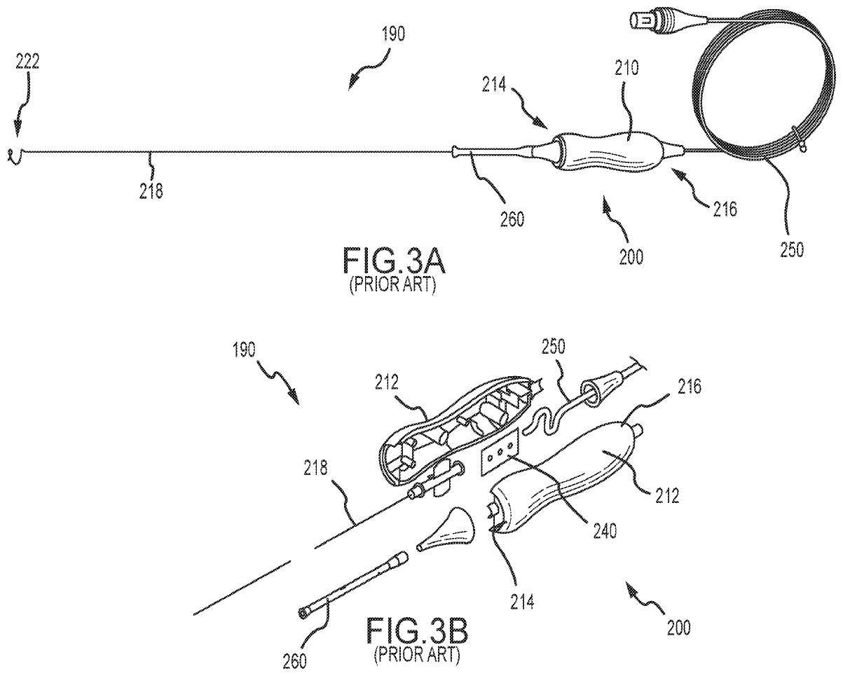

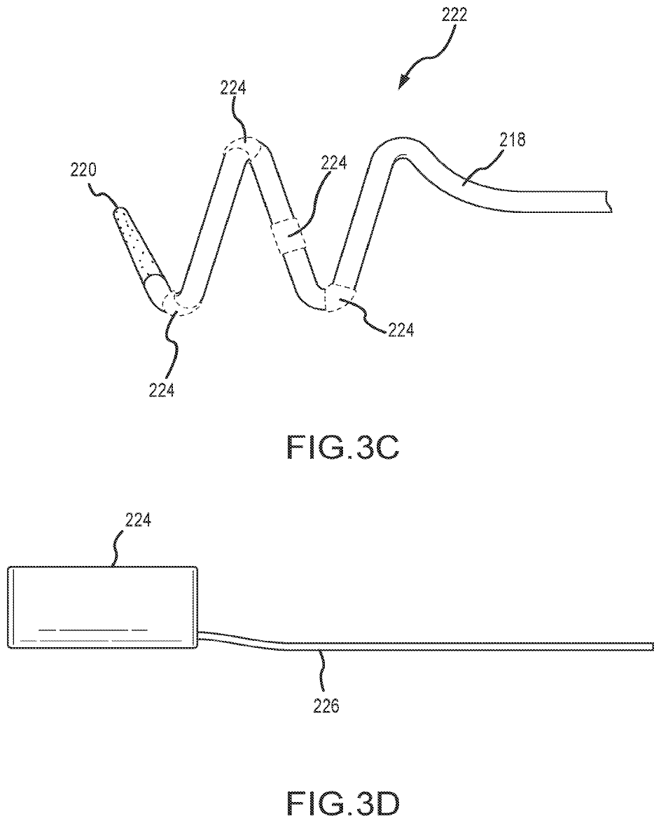

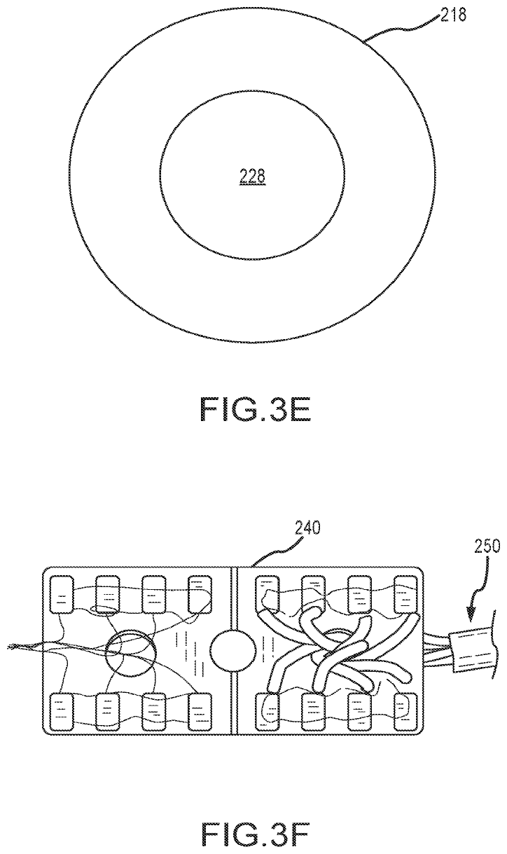

What may be referred to as a neuromodulation or denervation catheter assembly is illustrated in FIGS. 3A and 3B and is identified by reference numeral 190. The catheter assembly 190 includes a catheter 290 and a straightening tool 260. Individual components of the catheter assembly 190 will now be addressed.

The catheter 200 of FIGS. 3A and 3B may be used in place of the neuromodulation/denervation catheter 102 shown in FIG. 1A (or any other appropriate neuromodulation and denervation system). In any case the catheter 200 includes a handle 210 (e.g., a pair of detachably connected handle sections 212), a catheter shaft 218, a printed circuit board 240 or the like within the catheter handle 210, and a generator cable 250 (e.g., electrically/operatively interconnecting the catheter 200 with a console/generator of a neuromodulation/denervation system). The catheter handle 210 may be ergonomically shaped, and includes a distal end 214 and a proximal end 216.

A proximal portion of the catheter shaft 218 is disposed within the catheter handle 210, with the catheter shaft 218 extending distally of the distal end 214 of the catheter handle 210. The catheter shaft 218 includes a distal end section 222 that is also shown in FIG. 3C. The distal end section 222 of the catheter shaft 218 shown in FIGS. 3A and 3C is in its deployed or expanded state. A length dimension of the distal end section 222 may be described as proceeding in a helical or spiral pattern/configuration (and thereby is curved proceeding along its length dimension).

A plurality of electrodes 224 are spaced along the length of the distal end section 222. Each electrode 224 may be in the form of an annular band. In any case and as shown in FIG. 3D, one or more wires 226 extend from each electrode 224 back to the printed circuit board 240 that is enclosed within the catheter handle 210. In this regard, a lumen 228 is shown in FIG. 3E, and this lumen 228 extends from a distal end 220 of the catheter shaft 218 and through the catheter shaft 218 to accommodate electrically/operatively interconnecting the corresponding electrode(s) 224 with the printed circuit board 240. The printed circuit board 240 may include one or more bonding pads to provide an electrical/operative connection between the wires 226 for the electrodes 224 and the generator cable 250 (e.g., FIG. 3F). Any appropriate structure for providing an electrical/operative connection between the wires 226 for the electrodes 224 and the generator cable 250 may be utilized by the catheter handle 210.

A representative configuration of the catheter assembly 190 of FIGS. 3A-3F is shown in FIG. 4A, where its neuromodulation/denervation catheter is identified by reference numeral 220a. The neuromodulation/denervation catheter 220a incorporates a working length for the catheter shaft 218a that has been adapted, however, for a femoral access to a patient's vasculature (e.g., a working length of about 117 cm with the straightening tool 260). Another representative configuration of the catheter assembly 190 of FIGS. 3A-3F is shown in FIG. 4B, where its neuromodulation/denervation catheter is identified by reference numeral 220b. The neuromodulation/denervation catheter 220b incorporates a working length for the catheter shaft 218b, however, that has been adapted for a radial access to a patient's vasculature (e.g., a working length of greater than about 142 cm with the straightening tool 260). Note that the working length of the catheter shaft 218b (radial access) in FIG. 4B is longer than the working length of the catheter shaft 218a (femoral access) in FIG. 4A. Although the neuromodulation/denervation catheter 220b could be used to introduce the catheter shaft 218b through a femoral access, this would position the catheter handle 210 further from the femoral access and that could present one or more issues for an operator of the neuromodulation/denervation catheter 220b.

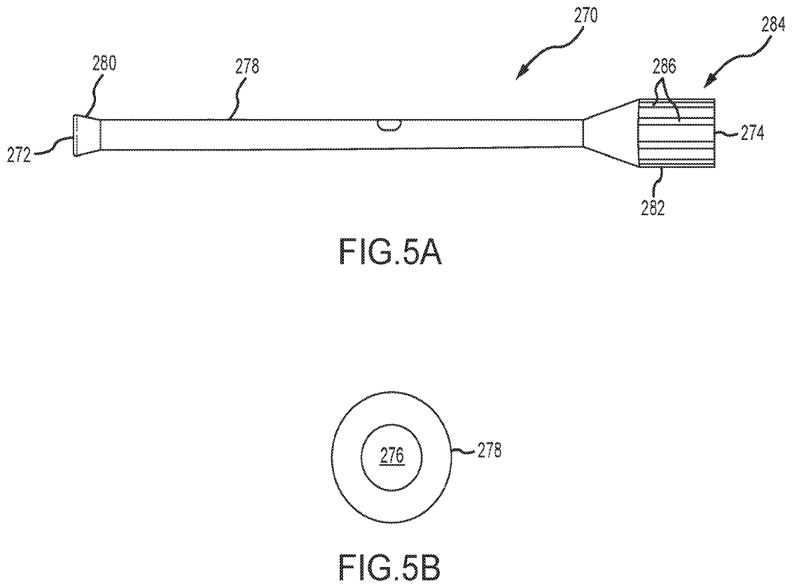

One embodiment of a multi-function catheter shaft tool (or a catheter shaft actuator) is shown in FIGS. 5A and 5B and is identified by reference numeral 270. Hereafter, the tool 270 will be addressed in relation to the neuromodulation/denervation catheter 200 of FIGS. 3A-3F (the multi-function catheter shaft tool 270 replacing the straightening tool 260). However, it should be appreciated the multi-function catheter shaft tool 270 may be used with any appropriate catheter that includes at least one length segment for its catheter shaft where it would be beneficial to move this length segment at least toward a “straighter” profile utilizing the tool 270, and including at some point in time prior to introducing the catheter shaft into the patient's vasculature. The tool 270 could be used with a catheter having a handle (including where the tool 270 could be detachably connectable to and detachably removable from the handle), or the tool 270 could be used without any catheter handle.

The multi-function catheter shaft tool 270 includes a distal end 272 and a proximal end 274 that are spaced from one another along a length dimension of the tool 270. The tool 270 may be of any appropriate length. A lumen 276 extends through an entirety of the length of the tool 270 (e.g., from its distal end 272 to its proximal end 274). This allows the tool 270 to be disposed anywhere along the length dimension of the catheter shaft 218, including where the catheter shaft 218 extends proximally of the tool 270. The cross-sectional shape of the lumen 276 may be constant throughout the length of the tool 270 although such is not required. The lumen 276 merely needs to accommodate passage of the catheter shaft 218 therethrough, and as such at least a portion of the lumen 276 need not necessarily be defined by an annular inner wall along the entirety of its length.

The multi-function catheter shaft tool 270 includes a catheter shaft straightener 278 and a catheter shaft actuator 282. The catheter shaft actuator 282 defines a proximal end section of the tool 270 in the illustrated embodiment, although it may be disposed anywhere along the length of the tool 270 so long as a sufficient length of the catheter shaft straightener 278 extends distally from the catheter shaft actuator 282. The catheter shaft straightener 278 extends distally from a distal end of the catheter shaft actuator 282 to the distal end 272 of the tool 270 in the illustrated embodiment. The catheter shaft straightener 278 may be of any appropriate length.

The catheter shaft straightener 278 may include a flared distal end section 280. An outer diameter of the distal end section 280 may progressively increase proceeding in the direction of the distal end 272 of the multi-function catheter shaft tool 270 (as may the inner diameter of the lumen 276 along the flared distal end section 280). A remainder of the catheter shaft straightener 279 may be of a constant outer diameter (e.g., a smaller outer diameter compared to a maximum outer diameter of the distal end section 280). The distal end section 280 could also be of the same diameter as the remainder of the catheter shaft straightener 278, but could be “softer” than a remainder of the catheter shaft straightener 278 (e.g., for enhanced manipulation of the distal end section 280, for instance when loading a guidewire into a catheter shaft). For instance, a durometer rating of the distal end section 280 (whether flared or not) may be less than a durometer rating of the remainder of the catheter shaft straightener 278 (e.g., the distal end section 280 could have a lower rating on the Shore Hardness Scale compared to a remainder of the catheter shaft straightener 278). The catheter shaft straightener 278 may be formed from any appropriate material or combination of materials, for instance silicone (e.g., to reduce a potential for damaging the electrodes 224 incorporated by the distal end section 222 of the catheter shaft 218).

The catheter shaft actuator 282 may include a grip or engagement section 284 having a larger outer diameter than an outer diameter of the catheter shaft straightener 278, for instance so as to be sufficiently sized for a user to exert a force on the catheter shaft actuator 282 by grasping the engagement section 284. The outer perimeter of the engagement section 284 may include a plurality of circumferentially-spaced segments 284 for facilitating engagement by a user. Any appropriate material or combination of materials may be used to fabricate/form the catheter shaft actuator 282. In one embodiment at least a portion of the catheter shaft actuator 282 is more rigid than the catheter shaft straightener 282 (e.g., at least part of the catheter shaft actuator 282 may have a higher durometer rating than the catheter shaft straightener 278). A distal section 288 of the catheter shaft actuator 282 extends distally from the engagement section 284, and may do so in converging relation to the lumen 276 proceeding in the direction of the catheter shaft straightener 278. In any case, at least part of the catheter shaft straightener 278 extends distally from a distal end of the catheter shaft actuator 282. The catheter shaft straightener 278 and catheter shaft actuator 282 are appropriately interconnected (e.g., fixed or joined) such that the catheter shaft straightener 278 and catheter shaft actuator 282 collectively rotate about a centerline of the lumen 276 (e.g., when torqueing the catheter shaft 218), when advancing the catheter shaft 218 within a patient's vasculature, or both.

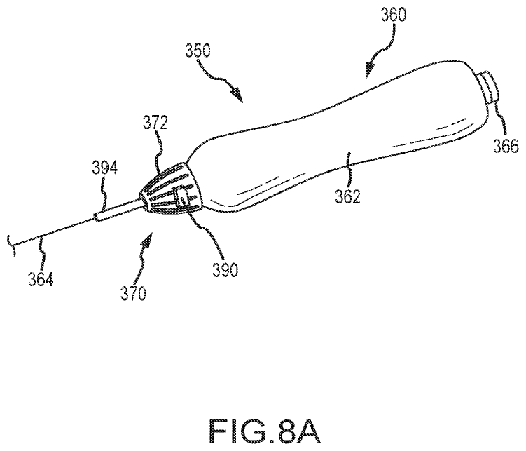

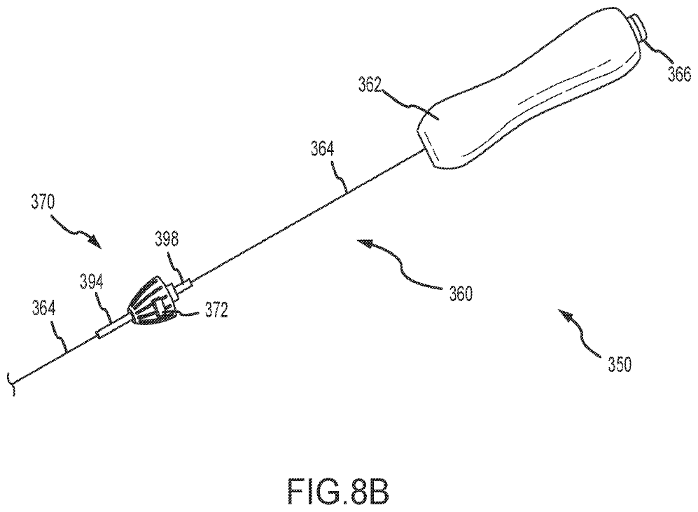

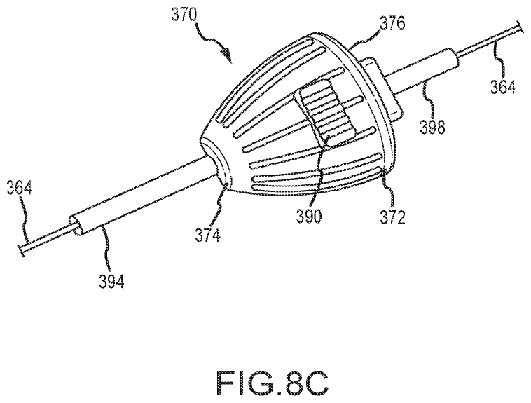

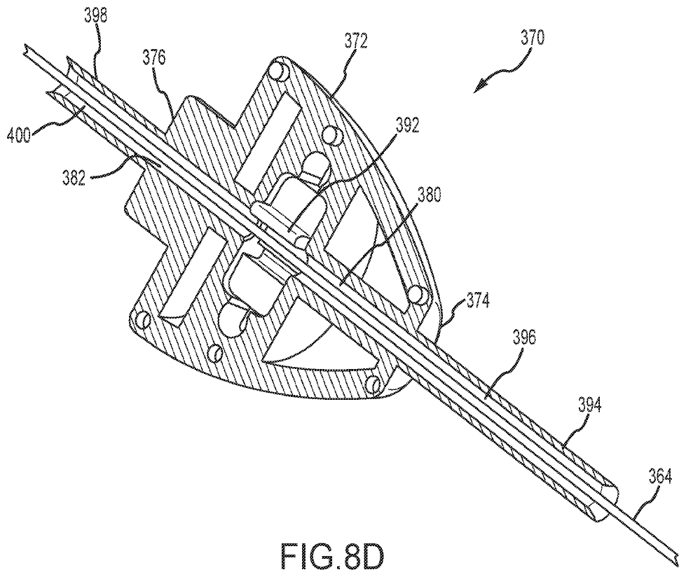

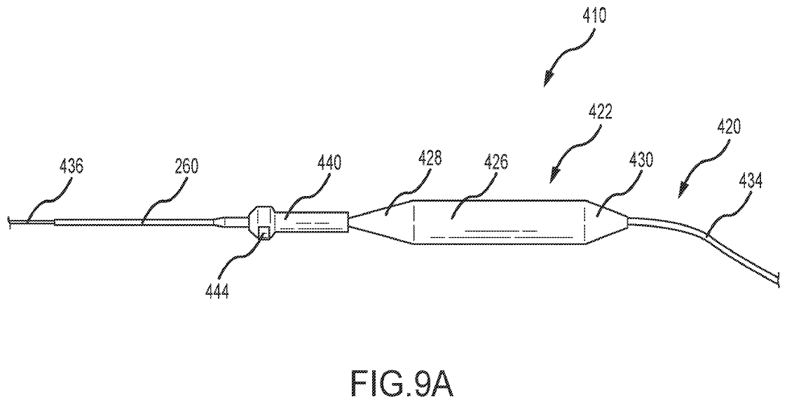

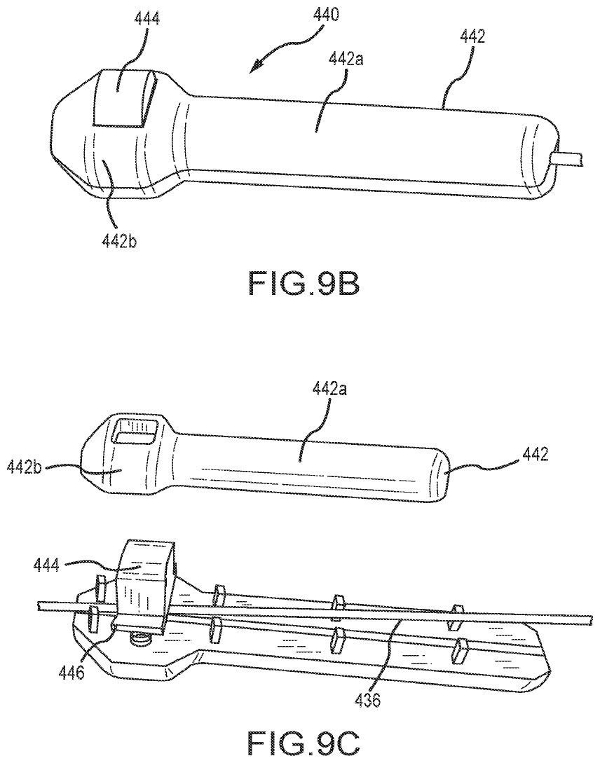

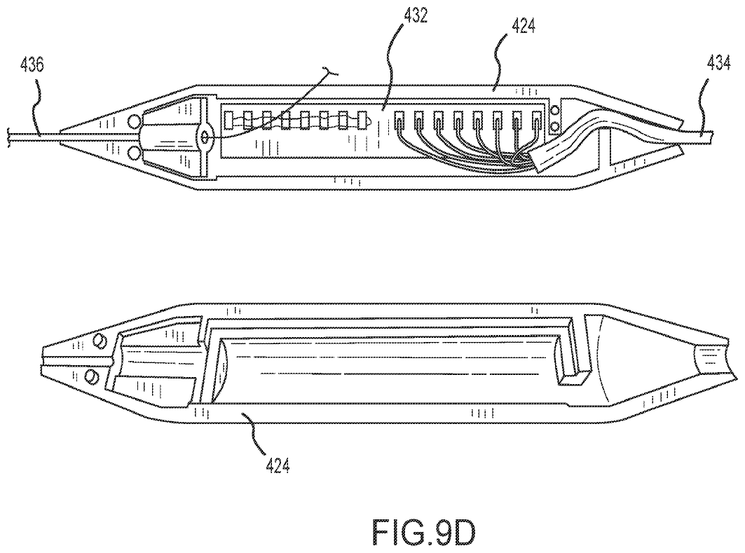

The catheter shaft actuator 282 may include an appropriate clamping mechanism that may be actuated between a released configuration and a clamping configuration. For instance, the multi-function catheter shaft tool 270 could include a clamping actuator 390 and clamp 392 of the type discussed below in relation to FIGS. 8A-8D, including where the actuator 390 is disposed on an exterior of the catheter shaft actuator 282 (or any other appropriate location). The clamping actuator 444 and corresponding clamp 446, discussed below in relation to FIGS. 9A-9D, could also be utilized by the multi-function catheter shaft tool 270. In any case disposing the clamping mechanism of the multi-function catheter shaft tool 270 in its released configuration allows the tool 270 to be repositioned along the length of the catheter shaft 218, whereas disposing the clamping mechanism of the multi-function catheter shaft tool 270 in its clamping configuration allows the tool 270 to suitably engage (e.g., clamp onto) the exterior of the catheter shaft 218 such that exerting a rotary movement on the catheter shaft actuator 282 may be used to torque the catheter shaft 218, such that an at least generally axially-directed force may be exerted on the catheter shaft actuator 282 (e.g., pushing or pulling) to advance the catheter shaft 218 relative to the patient's vasculature, or both.

The multi-function catheter shaft tool 270 of FIGS. 5A-5B provides two discrete functions—“straightening” (e.g., distal end section 222 of the catheter shaft 218) and movement of the catheter shaft 218 (e.g. torqueing and/or advancement relative to the patient's vasculature). The clamping mechanism of the multi-function catheter shaft tool 270 may be disposed in its released configuration such that the tool 270 may be advanced along the length of the catheter shaft 218 and in the direction of its distal end 214. The flared distal end section 280 of the catheter shaft straightener 278 should facilitate entry of the distal end section 222 of the catheter 218 (initially a proximal end of the distal end section 222) into the catheter shaft straightener 278. In this regard, the wall thickness of the flared distal end section 280 may be less than the wall thickness of the remainder of the catheter shaft straightener 278. Advancing the tool 270 in the distal direction should reduce the profile of the distal end section 222 of the catheter shaft 218 as it enters the catheter shaft straightener 278 (e.g., inwardly compress or move the distal end section 222 at least toward a “straighter” profile in relation to its length dimension). The distal end 272 of the tool 270 may be disposed at/proximate to the distal end 214 of the catheter shaft 218. At this time, the clamping mechanism of the multi-function catheter shaft tool 270 may be actuated into its clamping configuration, if desired. In any case, a proximal end of a guidewire (e.g., guidewire 140) may be directed into the lumen 228 that intersects with the distal end 220 of the catheter shaft 218. The guidewire may be advanced proximally a sufficient distance relative to the tool 270/catheter shaft 218, at which time the clamping mechanism of the multi-function catheter shaft tool 270 may be actuated to its released configuration such that the multi-function catheter shaft tool 270 may be advanced proximally along and relative to the catheter shaft 218 to an appropriate location.

The multi-function catheter shaft tool 270 may also be used to torque the catheter shaft 218 and/or to advance the catheter shaft 218 relative to the patient's vasculature. The clamping mechanism of the multi-function catheter shaft tool 270 may be actuated into its released configuration such that the multi-function catheter shaft tool 270 may be advanced along and relative to the catheter shaft 218 to a desired location (e.g., in proximity to the access to the patient's vasculature, whether using a femoral access, a radial access, or any other vasculature access). This may be characterized as changing the working length of the catheter shaft 218—the spacing between the distal end 272 of the multi-function catheter shaft tool 270 and the distal end 220 of the catheter shaft 218. Thereafter, the clamping mechanism of the multi-function catheter shaft tool 270 may be actuated into its clamping configuration such that the clamping mechanism of the multi-function catheter shaft tool 270 engages an exterior of the catheter shaft 218, where subsequent rotation of the catheter shaft actuator 282 (e.g., about a center line of the lumen 276) will correspondingly rotate the catheter shaft 218 about its length dimension and where advancement of the catheter shaft actuator 282 (e.g., by pushing or pulling on the catheter shaft actuator 282) will correspondingly advance the catheter shaft relative to the patient's vasculature. As the catheter shaft straightener 278 is appropriately fixed relative to the catheter shaft actuator 282, this movement of the catheter shaft actuator 282 will also correspondingly move the catheter shaft straightener 278.

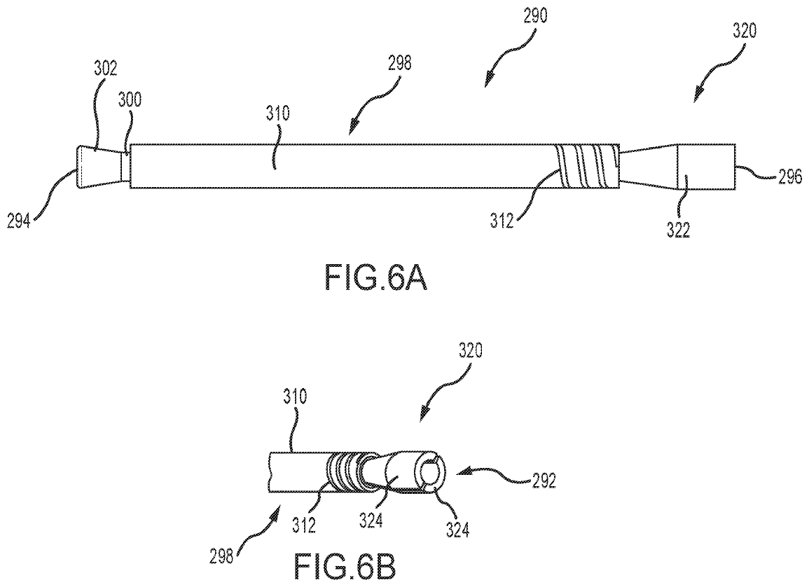

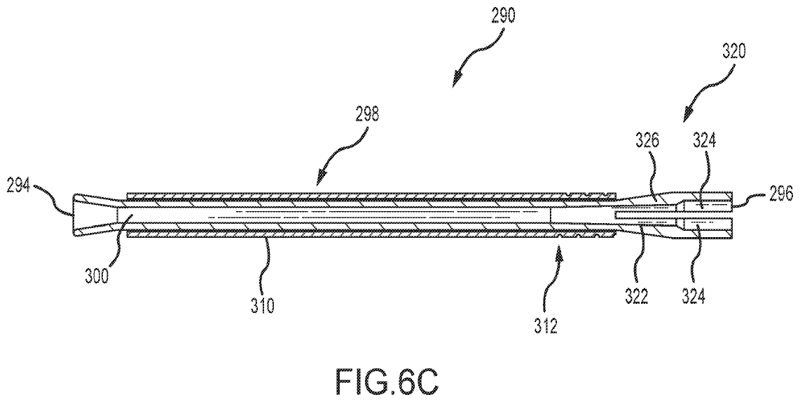

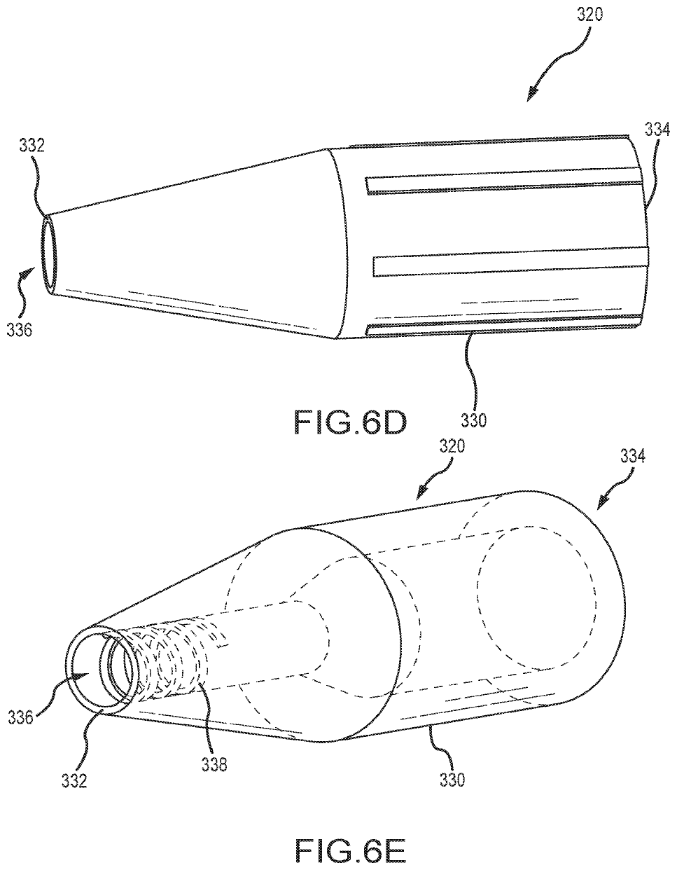

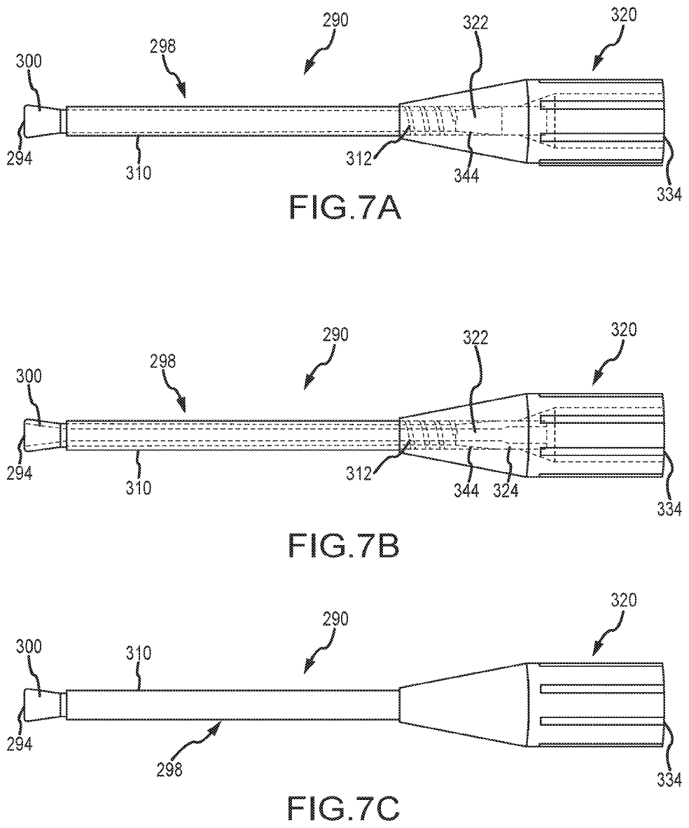

Another embodiment of a multi-function catheter shaft tool (or a catheter shaft actuator) is illustrated in FIGS. 6A-7C and is identified by reference numeral 290. Hereafter, the tool 290 will be addressed in relation to the neuromodulation/denervation catheter 200 of FIGS. 3A-3F (the multi-function catheter shaft tool 290 replacing the straightening tool 260—). However, it should be appreciated the multi-function catheter shaft tool 290 may be used with any appropriate catheter that includes at least one length segment for its catheter shaft where it would be beneficial to move this length segment at least toward a “straighter” profile utilizing the tool 290, and including at some point in time prior to introducing the catheter shaft into the patient's vasculature. The tool 290 could be used with a catheter having a handle (including where the tool 290 could be detachably connectable to and detachably removable from the handle), or the tool 290 could be used without any catheter handle.

The multi-function catheter shaft tool 290 includes a distal end 294 and a proximal end 296 that are shown in FIGS. 6A and 6C. An interior lumen 292 extends between this distal end 294 and proximal end 296. The distal end 294 is the distalmost end for the entirety of the tool 290, whereas the proximal end 296 is disposed at an intermediate location along a length dimension of the tool 290 and as will be addressed in more detail below.

There are two main components/subassemblies of the multi-function catheter shaft tool 290—a catheter shaft straightener 298 and a catheter shaft actuator 320. Details of the catheter shaft straightener 290 will be addressed in relation to FIGS. 6A-6C, which also illustrate a clamping section 322 of the catheter shaft actuator 320. The above-noted distal end 294 is disposed at the distal end of the catheter shaft straightener 290, while the above-noted proximal end 296 is disposed at the distal end of the clamping section 322 of the catheter shaft actuator 320.

The catheter shaft straightener 298 includes a liner 300 and an outer housing or tube 310. The liner 300 extends distally beyond a distal end of the outer housing 310, and may include a flared distal end section 302, which may be in accord with the distal end section 280 discussed above. An outer diameter of the distal end section 302 may progressively increase proceeding in the direction of the distal end 294 of the tool 290. A remainder of the liner 300 may be of a constant outer diameter (e.g., a smaller outer diameter compared to a maximum outer diameter of the distal end section 302). The liner 300 may be formed from any appropriate material or combination of materials, for instance silicone (e.g., to reduce a potential for damaging the electrodes 224 incorporated by the distal end section 222 of the catheter shaft 218).

The outer housing or tube 310 of the catheter shaft straightener 298 is disposed circumferentially about the liner 300 along a substantial portion of a length of the liner 300. The distal end of the outer housing 310 may be located adjacent to the flared distal end section 302 of the liner 300. External threads 312 are formed on an exterior of the outer housing 310. These threads 312 may be disposed on a proximal end section of the exterior of the outer housing 310.

A proximal end of the liner 300 may terminate within an interior location along the length of the outer housing 310. In any case, the proximal end of the liner 300 may be appropriately fixed or secured to the clamping section 322 of the catheter shaft actuator 320. At least one deflectable section 324 is incorporated on a proximal end section of the clamping section 322. A pair of deflectable sections 324 are disposed in opposing relation to one another in the illustrated embodiment, with a pair of opposing slots being disposed between this pair of deflectable sections 324. A cover 326 may be disposed over/about the liner 300 and clamping section 322 along their respective lengths.

Each of the liner 300 and the outer housing 310 may be formed from any appropriate material or combination of materials. However, the outer housing 310 may be more rigid than the liner 300 (e.g., for reducing the potential for damage to the electrodes 224 on the distal end section 222 of the catheter shaft 218). For instance, the liner 300 may formed from silicone or other suitably “soft” materials. The outer housing 310 may be characterized as having a first hardness, the liner 300 may be characterized as having a second hardness, with the second hardness being less than the first hardness, and that may facilitate reducing the profile of the distal end section 222 of the catheter shaft 218 while reducing the potential for damaging the electrodes 224 on the distal end section 222 of the catheter shaft 218 while introducing the distal end section 222 into the catheter shaft straightener 298 (e.g., the outer housing 310 may have a higher durometer rating than the liner 300).

FIGS. 6D and 6E present views of a body or cap 330 for the catheter shaft actuator 320. As the cap 330 may be used to change the catheter shaft actuator 320 between a clamping configuration and a released configuration (relative to the catheter shaft 218), as well as for torqueing and/or the catheter shaft 218, the cap 300 may also be referred to as a “catheter shaft actuator 330.” In any case, the cap 330 includes a distal end 322 and a proximal end 334 that are spaced from one another along a length dimension of the cap 330. A passage, open space, or pathway 336 extends from the distal end 332 of the cap 330 to the proximal end 334 of the cap 330. Internal threads 338 are formed about the passage 336 of the cap 330, for instance along a distal end section of the passage 336. An activating surface 340 is also disposed within the interior of the cap 330, interfaces with the passage 336, and cooperates with the clamping section 322 to dispose the catheter shaft actuator 320 into its clamping configuration. Based upon the foregoing it should be appreciated that the multi-function catheter shaft tool 290 may be mounted on the catheter shaft 218, with the catheter shaft 218 extending entirely through the length of the tool 290 (including proximally).

A proximal end section of the outer housing 310 of the catheter shaft straightener 298 may be directed into the passage 336 at the distal end 332 of the cap 330 (which also directs at least a proximal end portion of the clamping section 322 of the catheter shaft actuator 320 into this passage 336 as well). Rotation of the cap 330 (catheter shaft actuator 320) relative to the outer housing 310 (catheter shaft straightener 298) engages the interior threads 338 of the cap 330 with the external threads 312 of the outer housing 310, to thereby couple the catheter shaft actuator 280 to the catheter shaft straightener 298. The initial threaded engagement between the cap 330 and the outer housing 310 may allow for coupling of the catheter shaft actuator 320 and the catheter shaft straightener 298 without activating the deflectable sections 324 of the clamping section 322 (e.g., the deflectable sections 324 of the clamping section 322 may be spaced from or otherwise may not be exerting a clamping force on the catheter shaft 218 at this time). This again will be referred to as a “released configuration” for the catheter shaft actuator 320. It should be appreciated that the catheter shaft actuator 320 is also in its released configuration when the cap 330 is not threadably engaged with the outer housing 310. When a threaded engagement has been established between the cap 330 and the outer housing 310, continued rotation of the cap 330 relative to the outer housing 310 will axially advance the cap 330 in the direction of the distal end 294 of the tool 290, which will eventually cause the activating surface 340 of the cap 330 to exert an inwardly-directed force on the deflectable sections 324 of the clamping section 322, so as to dispose the clamping section 322 in the clamping configuration where at least the deflectable sections 324 are clamped against an exterior of the catheter shaft 218.

The multi-function catheter shaft tool 290 of FIGS. 6A-7C provides two discrete functions—“straightening” (e.g., distal end section 222 of the catheter shaft 218) and movement of the catheter shaft 218 (e.g. torqueing and/or advancement relative to the patient's vasculature). The catheter shaft actuator 320 may be disposed in its released configuration such that the tool 290 may be advanced along the length of the catheter shaft 218 and in the direction of its distal end 214 of the catheter shaft 218. The flared distal end section 302 of the liner 300 should facilitate entry of the distal end section 222 of the catheter 218 (initially a proximal end of the distal end section 222) into the liner 300. In this regard, the wall thickness of the flared distal end section 302 may be less than the wall thickness of the remainder of the liner 300. Advancing the tool 290 in a distal direction should reduce the profile of the distal end section 222 of the catheter shaft 218 as it enters the catheter shaft straightener 298 (e.g., inwardly compress or move the distal end section 222 at least toward a “straighter” profile in relation to its length dimension). The distal end 294 of the tool 290 may be disposed at/proximate to the distal end 214 of the catheter shaft 218. At this time, the catheter shaft actuator 320 may be actuated into its clamping configuration in the above-manner, if desired. In any case, a proximal end of a guidewire (e.g., guidewire 140) may be directed into the lumen 228 that intersects with the distal end 220 of the catheter shaft 218. The guidewire may be advanced proximally a sufficient distance relative to the tool 290/catheter shaft 218, at which time the catheter shaft actuator 320 may be actuated to its released configuration such that the multi-function catheter shaft tool 290 may be advanced proximally along and relative to the catheter shaft 218 to an appropriate location.

The multi-function catheter shaft tool 290 may also be used to torque the catheter shaft 218 and/or to advance the catheter shaft 218 relative to the patient's vasculature. The catheter shaft actuator 320 may be actuated into its released configuration such that the multi-function catheter shaft tool 290 may be advanced along and relative to the catheter shaft 218 to a desired location (e.g., in proximity to the access to the patient's vasculature, whether using a femoral access, a radial access, or any other vasculature access). This may be characterized as changing the working length of the catheter shaft 218—the spacing between the distal end 294 of the multi-function catheter shaft tool 290 and the distal end 220 of the catheter shaft 218. Thereafter, the catheter shaft actuator 320 may be actuated into its clamping configuration such that catheter shaft actuator 320 (more specifically the deflectable sections 324 of its clamping section 322) engages an exterior of the catheter shaft 218. At this time, rotation of the catheter shaft actuator 320 (e.g., about a centerline of the lumen 292) will correspondingly rotate the catheter shaft 218 about its length dimension. As the catheter shaft straightener 298 is appropriately fixed relative to the catheter shaft actuator 320, this rotation of the catheter shaft actuator 320 will also correspondingly rotate the catheter shaft straightener 298. Advancement of the catheter shaft actuator 320 (e.g., by pushing or pulling on the catheter shaft actuator 320) will advance the catheter shaft 218 relative to the patient's vasculature in a corresponding direction (and will similarly advance the catheter shaft straightener 298).

An embodiment of a catheter assembly is illustrated in FIGS. 8A-8D and is identified by reference numeral 350. There are two primary components/subassemblies of the catheter assembly 350—a catheter 360 and a catheter shaft actuator 370. The catheter 360 may correspond with the above-noted neuromodulation/denervation catheter 200, but the catheter 360 may be of any appropriate type/configuration and that provides any appropriate function or combination of functions when disposed in a patient's vasculature (e.g., diagnostic, therapeutic, neuromodulation, denervation).

The catheter 360 includes a handle 362 and a catheter shaft 364 that extends distally from a distal end of the catheter handle 362. The catheter shaft 364 may extend at least into the distal end of the catheter shaft handle 362, and the catheter shaft handle 362 may engage the catheter shaft 364 in a manner that allows the catheter shaft handle 362 to be used to move the catheter shaft 364 (e.g., torque and/or advance the catheter shaft 364 relative to the patient's vasculature) in a manner that will be discussed in more detail below (e.g. the catheter shaft 364 may be appropriately anchored/fixed relative to the catheter handle 362). A generator connector 366 extends from a proximal end of the catheter handle 360.

The catheter shaft actuator 370 includes a body 372 having a distal end 374 and a proximal end 376 that are spaced from one another along a length dimension of the body 372. As the body 372 may be used to move the catheter shaft 218 in a manner that will be discussed in more detail below, the body 372 may also be referred to as a “catheter shaft actuator 372.” The body 372 may be fabricated in any manner, for instance in separate halves.

An annular first tube 394 extends from the distal end 374 of the body 372. This first tube 394 may be used to reduce the potential for kinking of the catheter shaft 218 when being torqued and/or advanced (e.g., relative to the patient's vasculature) by the catheter shaft actuator 370. An annular second tube 398 extends from the proximal end 376 of the body 372. The first tube 394 includes an annular first lumen 396 that extends to a passage defined by an annular first cylindrical section 380 within the interior of the body 372. The second tube 398 includes an annular second lumen 400 that extends to a passage defined by an annular second cylindrical section 382 within the interior of the body 372. The first cylindrical section 380 and the second cylindrical section 382 are axially aligned but are spaced from one another within the interior of the body 372. A clamp 392 is disposed in the space between the first cylindrical section 380 and the second cylindrical section 382 within the interior of the body 372. An actuator 390 is disposed on an exterior of the body 372 and is movable between first and second positions to dispose the clamp in a released configuration and a clamping configuration, respectively. As such, the actuator 390 may also be referred to as a “clamping actuator 390.”

Disposing the clamping actuator 390 in its released configuration (by movement of the actuator 390, to produce a corresponding movement of the clamp 392) allows the catheter shaft actuator 370 to be advanced along and relative to the catheter shaft 364 to a desired location (e.g., in proximity to the access to the patient's vasculature, whether using a femoral access, a radial access, or any other vasculature access). This may be characterized as changing the working length of the catheter shaft 364—the spacing between the distal end 374 of the catheter shaft actuator 370 and the distal end of the catheter shaft 364. Disposing the clamping actuator 390 in its clamping configuration (by movement of the actuator 390, to produce a corresponding movement of the clamp 392) causes the clamp 392 to engage an exterior of the catheter shaft 364 such that rotation of the catheter shaft actuator 370 (e.g., by engagement of the body 372) will correspondingly rotate the catheter shaft 364 about its length dimension, and such that advancement of the catheter shaft actuator 370 (e.g., by engagement of and pushing or pulling on the body 372) will advance the catheter shaft 364 along the patient's vasculature in the corresponding direction. The noted “rotation” may be described as being about a centerline through the first lumen 396 of the first tube 394, the passage defined by the first cylindrical section 380, the passage defined by the second cylindrical section 382, and the second lumen 400 of the second tube 398.

The catheter shaft actuator 370 may be disposed such that its second tube 398 extends within the distal end of the catheter handle 362 and as shown in FIG. 8A. Disposing the clamping actuator 390 in its clamping configuration (by movement of the actuator 390, to produce a corresponding movement of the clamp 392) may maintain the catheter shaft actuator 370 in a fixed position relative to the catheter shaft handle 364 at this time. Features could also be incorporated to actually detachably connect the catheter handle 362 and the catheter shaft actuator 370. In any case and in the FIG. 8A configuration, the catheter shaft handle 364 may be used to torque the catheter shaft 364 (e.g., by a user engaging the catheter handle 362 and rotating the same about the length dimension of the catheter shaft 364), to advance the catheter shaft 364 (e.g., by a user engaging the catheter handle 362 and pushing or pulling on the catheter handle 364 to advance the catheter shaft 364 along the patient's vasculature in a corresponding direction), or both. For instance, the configuration shown in FIG. 8A could be for a radial access to the patient's vasculature (or any other vasculature access). Having the clamping actuator 390 in its clamping configuration when disposed in the FIG. 8A configuration (even when the user is engaging the catheter handle 362 to exert a force on the catheter shaft 364), however, may also further facilitate the desired movement of the catheter shaft 364, as the catheter handle 362 and the clamp 392 would be engaging the catheter shaft 364 at spaced locations and each of these engagements alone should accommodate the desired movement of the catheter shaft 364 (whether via manipulation of the catheter shaft actuator 370 or the catheter handle 362).

The clamping actuator 390 may be disposed in its released configuration (by movement of the actuator 390, to produce a corresponding movement of the clamp 392) such that the catheter shaft actuator 370 may be advanced distally along the catheter shaft 364, to thereby position the catheter shaft actuator 370 closer to the vasculature access being utilized and where the catheter shaft actuator 370 is now spaced from the catheter handle 362, for instance as shown in FIG. 8B. Although such a configuration may be utilized for a femoral access to the patient's vasculature, it of course may also be utilized for a radial access to the patient's vasculature (or any other access to the patient's vasculature). Generally, the catheter shaft actuator 370 may be moved to any position along the catheter shaft 364, and thereafter the clamping actuator 390 may be disposed in its clamping configuration where the clamp 392 clampingly engages the exterior of the catheter shaft 364. In any case and with the clamping actuator 390 being in its clamped configuration, a user may engage the body 372 to exert a desired force or combination of forces on the catheter shaft 218 in the manner described herein and to yield a desired movement or combination of movements of the catheter shaft 218.

Another embodiment of a catheter assembly is illustrated in FIGS. 9A-9D and is identified by reference numeral 410. There are three primary components/subassemblies of the catheter assembly 410—a catheter 420, a catheter shaft actuator 440, and the above-described straightening tool 260.

The catheter 420 may be of any appropriate type/configuration and may provide any appropriate function or combination of functions when disposed in a patient's vasculature (e.g., diagnostic, therapeutic, neuromodulation, denervation). The catheter 420 includes an electrical housing 422 and a catheter shaft 436 that extends distally from a distal end of the electrical housing 422. The catheter shaft 436 may extend at least into the distal end of the electrical housing 422. A generator cable 434 extends proximally from a proximal end of the electrical housing 422.

The electrical housing 422 may be defined by a pair of housing sections 424 that are appropriately interconnected. A printed circuit board 432 is enclosed within the electrical housing 422. The printed circuit board 432 may include one or more bonding pads to provide an electrical/operative connection between the wires for any electrodes or other electrical components incorporated by the catheter shaft 436 (e.g., electrodes 224) and the generator cable 434. Any appropriate structure for providing an electrical/operative connection between the wires for the electrodes/electrical components of the catheter shaft 436 and the generator cable 434 may be utilized by the electrical housing 422.

In the case of the catheter assembly 410, the electrical housing 422 is not in the form of a traditional catheter handle. Instead the primary function of the electrical housing 422 is to enclose the printed circuit board 432 (or more generally the structure for providing an electrical/operative connection between the wires 226 for the electrodes 224 and the generator cable 434). The electrical housing 422 is subject to a number of characterizations in this regard. One is that the exterior of the housing 422 is not ergonomically shaped. Another is that a maximum outer diameter of the housing 422 (taken perpendicular to its length dimension) is reduced compared to typical catheter handles. In the illustrated embodiment, the housing 422 includes a cylindrical section 426, a distal end section 428, and a proximal end section 430, with the cylindrical section 426 being disposed between the distal end section 428 and the proximal end section 430, and with the distal end section 428 and the proximal end section 430 being spaced from one another along a length dimension of the housing 422. The distal end section 428 converges toward a centerline through the housing 422 (corresponding with the length dimension of the housing 422) proceeding in the distal direction from the cylindrical section 426. Similarly, the proximal end section 430 converges toward the centerline through the housing 422 proceeding in the proximal direction from the cylindrical section 426.

The catheter shaft actuator 440 is positioned on the distal side of the housing 422, includes a housing 442, an actuator 444, and a clamp 446, and provides the primary structure for moving the catheter shaft 436 in the case of the catheter assembly 410. The housing 442 includes a proximal housing section 442a and a distal housing section 442b. The distal housing section 442b incorporates the actuator 444 and the clamp 446. A user may grasp the proximal housing section 442a to move the catheter shaft 436 in the desired manner. As such, the proximal housing section 442a may also be referred to as a “catheter shaft actuator 442a.”

The actuator 444 is disposed on an exterior of the catheter shaft actuator 440 and is movable between first and second positions to dispose the clamp 446 in a released configuration and a clamping configuration, respectively. As such, the actuator 444 may also be referred to as a “clamping actuator 444.” Disposing the clamping actuator 444 in its released configuration (by movement of the actuator 444, to produce a corresponding movement of the clamp 446) allows the catheter shaft actuator 440 to be advanced along and relative to the catheter shaft 436 to a desired location (e.g., in proximity to the access to the patient's vasculature, whether using a femoral access, a radial access, or any other vasculature access). This may be characterized as changing the working length of the catheter shaft 436—the spacing between the distal end of the catheter shaft actuator 440 and the distal end of the catheter shaft 436. Disposing the clamping actuator 444 in its clamping configuration (by movement of the clamping actuator 444, to produce a corresponding movement of the clamp 446) allows the clamping actuator 444 (more specifically the clamp 446) to engage an exterior of the catheter shaft 436 such that rotation of the catheter shaft actuator 440 (e.g., about a centerline coinciding with a length dimension of the catheter shaft actuator 440, which coincides with the length of the catheter shaft 436 extending through the catheter shaft actuator) will correspondingly rotate the catheter shaft 436 about its length dimension, and such that advancement of the catheter shaft actuator 440 (e.g., by engagement of and pushing or pulling on the catheter shaft actuator 440) will advance the catheter shaft 436 along the patient's vasculature in the corresponding direction. Generally, the catheter shaft actuator 440 may be moved to any position along the catheter shaft 436 that is desired by the user, and thereafter with the clamping actuator 444 being in its clamped configuration, a user may engage the catheter shaft actuator 440 to exert a desired force or combination of forces on the catheter shaft 436 in the manner described herein and to yield a desired movement or combination of movements of the catheter shaft 436.

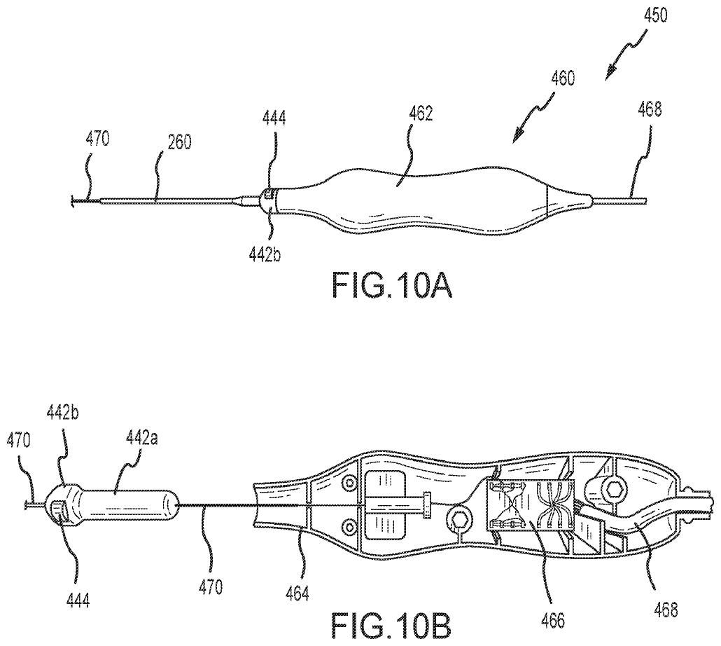

Another embodiment of a catheter assembly is illustrated in FIGS. 10A-10B and is identified by reference numeral 450. There are three primary components/subassemblies of the catheter assembly 450—a catheter 460, the above-described catheter shaft actuator 440, and the above-described straightening tool 260.

The catheter 460 may be of any appropriate type/configuration and may provide any appropriate function or combination of functions when disposed in a patient's vasculature (e.g., diagnostic, therapeutic, neuromodulation, denervation). The catheter 460 includes a catheter handle 462 and a catheter shaft 470 that extends distally from a distal end of the catheter handle 462. The catheter shaft 470 may extend at least into the distal end of the catheter handle 462, and may be of any appropriate configuration (e.g., in accord with the above-discussed catheter shaft 218). The catheter shaft 470 may be appropriately anchored/fixed relative to the catheter handle 462 to allow the catheter handle 462 to be used to torque the catheter shaft 470 (e.g., in the FIG. 10A configuration), to be used to advance the catheter handle 462 (e.g. by pushing or pulling on the catheter handle 462 to advance the catheter shaft 470 relative to the patient's vasculature in a corresponding direction), or both. A generator cable 468 extends proximally from a proximal end of the catheter handle 462.

The catheter handle 462 may be defined by a pair of handle sections 464 that are appropriately interconnected (only one handle section 464 being shown in FIG. 10B). A printed circuit board 466 is enclosed within the catheter handle 462. The printed circuit board 466 may include one or more bonding pads to provide an electrical/operative connection between the wires for any electrodes or other electrical components incorporated by the catheter shaft 470 (e.g., electrodes 224) and the generator cable 468. Any appropriate structure for providing an electrical/operative connection between the wires for the electrodes (or other electrical components incorporated by the catheter shaft 470) and the generator cable 468 may be utilized by the catheter handle 462.

The distal housing section 442b of the catheter shaft actuator 440 is disposed distally of the catheter handle 462 and the proximal housing section 442a is disposed within the catheter handle 462 in the FIG. 10A configuration (e.g., for radial access, or any other appropriate vasculature access). Again, the clamping actuator 444 is disposed on an exterior of the catheter shaft actuator 440 (specifically the distal housing section 442b) and is movable between first and second positions to again dispose the clamp 446 in a released configuration and a clamping configuration, respectively. Disposing the clamping actuator 444 in its released configuration (by movement of the actuator 444, to produce a corresponding movement of the clamp 446) allows the catheter shaft actuator 440 to be advanced along and relative to the catheter shaft 470 to a desired location (e.g., in proximity to the access to the patient's vasculature, whether using a femoral access, a radial access, or any other vasculature access). This may be characterized as changing the working length of the catheter shaft 470—the spacing between the distal end of the catheter shaft actuator 440 and the distal end of the catheter shaft 470. Disposing the clamping actuator 444 in its clamping configuration (by movement of the actuator 444, to produce a corresponding movement of the clamp 446) allows the clamping actuator 444 (more specifically the clamp 446) to engage an exterior of the catheter shaft 470 such that rotation of the catheter shaft actuator 440 (e.g., about a centerline coinciding with a length dimension of the catheter shaft actuator 440, which coincides with the length of the catheter shaft 436 extending through the catheter shaft actuator 440) will correspondingly rotate the catheter shaft 436 about its length dimension, and such that advancement of the catheter shaft actuator 440 (e.g., by engagement of and pushing or pulling on the catheter shaft actuator 440) will advance the catheter shaft 436 along the patient's vasculature in the corresponding direction. Generally, the catheter shaft actuator 440 may be moved to any position along the catheter shaft 470 that is desired by the user, and thereafter with the clamping actuator 444 being in its clamped configuration, a user may engage the catheter shaft actuator 440 to exert a desired force or combination of forces on the catheter shaft 436 in the manner described herein and to yield a desired movement or combination of movements of the catheter shaft 470.

Features may be incorporated to detachably connect the catheter handle 462 and the catheter shaft actuator 470, for instance when in the FIG. 10A configuration, although such is not required. In any case and in the FIG. 10A configuration, the catheter shaft handle 462 may be used to torque the catheter shaft 470 (e.g., by a user engaging the catheter handle 462 and rotating the same about the length dimension of the catheter shaft 470). For instance, the configuration shown in FIG. 10A could be for a radial access to the patient's vasculature. Disposing the clamping actuator 444 in its clamping configuration (by movement of the actuator 444, to produce a corresponding movement of the clamp 446), again allows the catheter shaft actuator 440 (more specifically the clamp 446) to engage an exterior of the catheter shaft 470. This may be used to maintain the catheter shaft actuator 440 in a fixed position relative to the catheter handle 462 for the FIG. 10A configuration. Having the clamping actuator 444 in its clamping configuration when the catheter shaft actuator 440 is disposed in the FIG. 10A configuration (even when the user is engaging the catheter handle 462 to exert a force on the catheter shaft 470), however, may also further facilitate the desired movement of the catheter shaft 470, as the catheter handle 462 and the clamp 444 would be engaging the catheter shaft 470 at spaced locations and each of these engagements alone should accommodate the desired movement of the catheter shaft 470 (whether via manipulation of the catheter shaft actuator 440 (specifically distal housing section 442b) or the catheter handle 462).

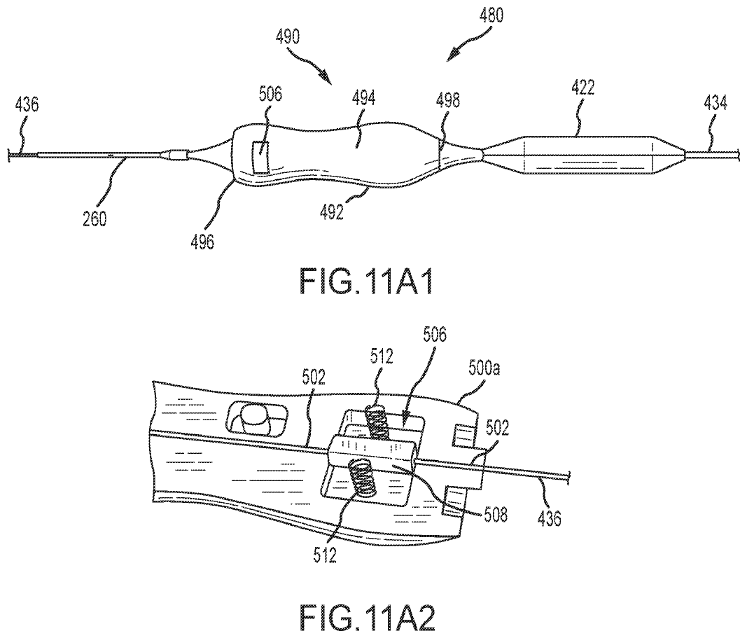

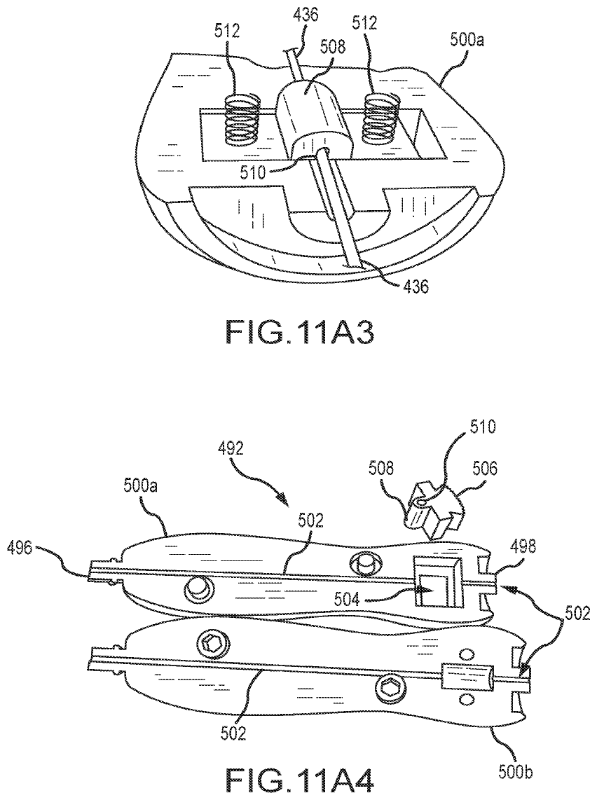

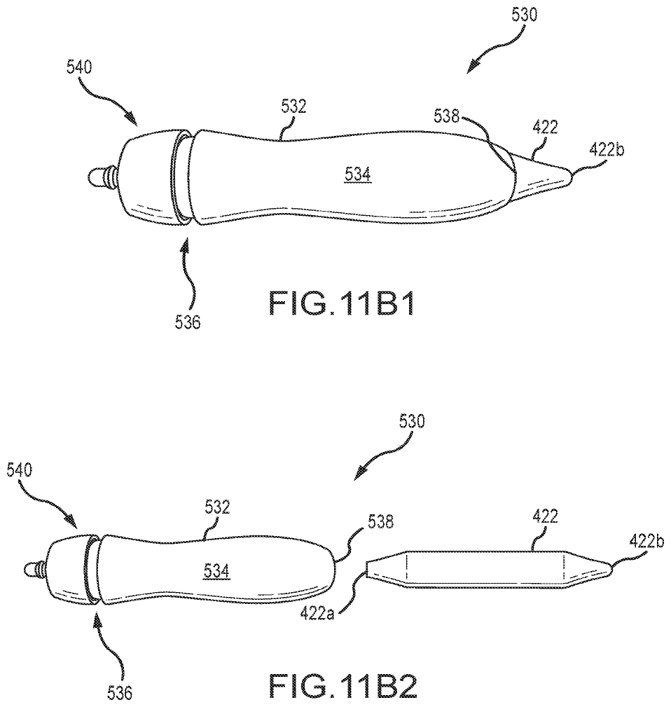

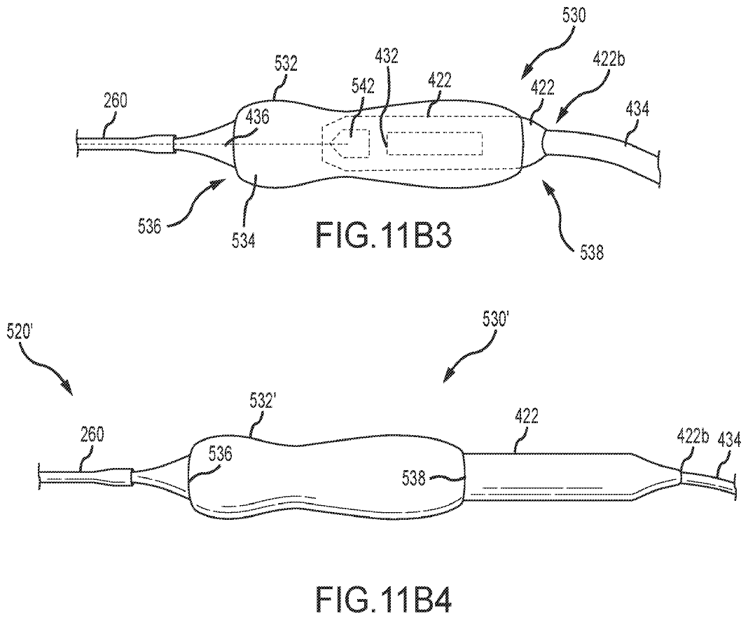

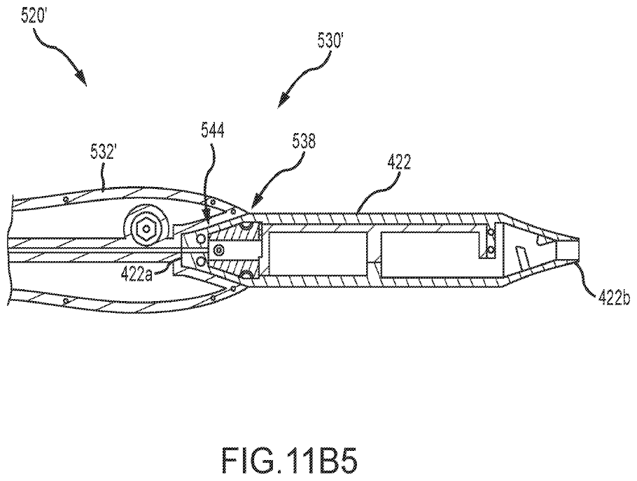

An embodiment of a catheter assembly is illustrated in FIGS. 11A1-11A4 and is identified by reference numeral 480. There are two primary components/subassemblies of the catheter assembly 480—a catheter 490 and the above-described straightening tool 260 (optional). The catheter 490 may be characterized as including a catheter handle or a catheter shaft actuator 492, the above-noted housing 422, the above-noted catheter shaft 436, and the above-noted generator cable 434. The catheter shaft 436 extends through the catheter handle 492 and at least into the housing 422. As noted above, the generator cable 434 extends proximally from a proximal end of the electrical housing 422.

The catheter handle 492 includes a body 494 that extends from a distal end 496 to a proximal end 498 of the catheter handle 492. A length dimension of the catheter handle 492 may be characterized as coinciding with a spacing between the distal end 496 and the proximal end 498. In any case, an outer perimeter of the catheter handle 492 (or its body 494) may be ergonomically-shaped for engagement by a user. For instance, the catheter handle 492 may include one more curved surfaces proceeding along its length dimension. An outer diameter of the catheter handle 492 may progressively increase proceeding from a first location (e.g., a location having a smallest effective outer diameter for the catheter handle 492; an intermediate location along the length of the catheter handle 492) and in the direction of (but not necessarily to) the proximal end 498. In the illustrated embodiment, a distal end section of catheter handle 492 (e.g., from the noted first location to the proximal end 498) may be characterized as being at least generally convexly-shaped relative to the length dimension of the catheter handle 492, which may facilitate engagement by a user. Other catheter handles described herein may utilize the described shape of the catheter handle 492.

The catheter shaft 436 extends distally from the distal end 496 of the catheter handle 492, and also extends proximally from the proximal end 498 of the catheter handle 492 and at least into the housing 422. The catheter handle 492 may be characterized as a catheter shaft actuator—a structure that can be used to manipulate the catheter shaft 436, such as torqueing the catheter shaft 436 and/or advancing the catheter shaft 436 relative to a patient's vasculature. That is, the catheter handle 492 may engage the catheter shaft 436 in a manner that allows the catheter handle 492 to be utilized by a user to move the catheter shaft 436 (e.g., torque and/or advance the catheter shaft 436 relative to the patient's vasculature). In this regard, the catheter shaft 436 may be appropriately anchored/fixed (e.g., detachably) relative to the catheter handle 492.

The body 494 of the catheter handle 492 may be defined by a pair of handle sections 500a, 500b that are appropriately interconnected (e.g., detachably). The handle section 500a includes a pocket 504 that movably receives a clamping actuator 506 that is accessible on an exterior of the catheter handle 492. A catheter shaft clamp 508 is appropriately interconnected with the clamping actuator 506. The catheter shaft clamp 508 and the clamping actuator 506 could be separate components that are attached to one another in any appropriate manner, or may be integrally formed as shown in the illustrated embodiment. In any case, a slot 510 extends through the length of the catheter shaft clamp 508 to allow the catheter shaft 436 to extend completely through the catheter shaft clamp 508 along its length dimension. A pair of catheter shaft supports 502 are incorporated by the handle section 500a and are disposed on opposite ends of the catheter shaft clamp 508.

A pair of springs 512 are disposed between/engage the handle section 500b and the catheter shaft clamp 508 and that bias the catheter shaft clamp 508 for a clamped engagement with the catheter shaft 436 (e.g., to limit/restrain relative movement between the catheter handle 492 and the catheter shaft 436). The force exerted by the catheter shaft clamp 508 on the catheter shaft 436 (a movement of the catheter shaft clamp 508 away from the handle section 500b) clamps the catheter shaft 436 against the two catheter shaft supports 502 of the handle section 500a (a “clamped configuration” for the catheter shaft clamp 508). This clamped engagement between the catheter shaft clamp 508 and the catheter shaft 436 may correspond with a home or first position for the clamping actuator 506. At this time, the catheter handle 492 may be used to move/manipulate the catheter shaft 436 relative to the patient's vasculature, as the catheter shaft 436 should be maintained in a fixed position relative to the catheter handle 492.

Moving the clamping actuator 506 from the noted first position to a second position (e.g., by engaging the portion of the clamping actuator 506 that protrudes beyond the body 494 of the catheter handle 492, and then depressing the clamping actuator 506 at least generally in the direction of the catheter shaft 436), releases the catheter shaft clamp 508 from the catheter shaft 436 (e.g., a released configuration for the catheter shaft clamp 508; by pushing the closed end of the slot 510 away from the catheter shaft 436) to allow the catheter handle 492 to be moved along and relative to the catheter shaft 436 to dispose the catheter handle 492 in a desired position relative to the catheter shaft 436 (e.g., to provide a desired working length for the catheter shaft 436). Typically it is the catheter handle 492 that is moved, with the catheter shaft 436 remaining stationary during this adjustment of the noted relative position. As such, the housing 422 should remain in a stationary position at this time as well (i.e., during movement of the catheter handle 492 relative to the catheter shaft 436). Once the catheter handle 492 is in the desired position along the length of the catheter shaft 436, the clamping actuator 506 may be released such that the catheter shaft clamp 508 moves from its second position back to its first position to once again exert a clamping force on the catheter shaft 436 (to dispose the catheter shaft clamp 508 back in its clamped configuration). It should be appreciated that the catheter handle 492 is disposed in its most proximal position, relative to the catheter shaft 436, in the view shown in FIG. 11A1. As such, the catheter handle 492 may be released from the catheter shaft 436 (by activating the clamping actuator 506 to dispose the catheter shaft clamp 508 in its released configuration) to allow the catheter handle 492 to be moved distally from the housing 422 and along the catheter shaft 436 from the view shown in FIG. 11A1.

An embodiment of a catheter assembly is illustrated in FIGS. 11B1-11B3 and is identified by reference numeral 520. The catheter assembly 520 includes a catheter 530 and may include the above-described straightening tool 260 (optional). The catheter 530 may be characterized as including a catheter shaft actuator or catheter handle 532, the above-noted housing 422, the above-noted catheter shaft 436, and the above-noted generator cable 434. The catheter shaft 436 extends through the catheter handle 532 (the catheter shaft 436 also extending distally of the catheter handle 532), through a distal end 422a of the housing 422, and is secured relative to the housing 422 via a catheter shaft anchor 542 (e.g., the housing 422 and the catheter shaft 436 should be maintained in a fixed position relative to one another). As noted above, the generator cable 434 extends proximally from a proximal end 422b of the electrical housing 422.

The catheter handle 532 includes a body 534 that extends from a distal end 536 to a proximal end 538 of the catheter handle 532. A length dimension of the catheter handle 532 may be characterized as coinciding with a spacing between the distal end 536 and the proximal end 538. In any case, an outer perimeter of the catheter handle 532 (or its body 534) may be ergonomically-shaped for engagement by a user (e.g., in accord with the catheter handle 492 shown in FIG. 11A1).

The catheter shaft 436 extends distally from the distal end 536 of the catheter handle 532. The catheter handle 532 may be characterized as a catheter shaft actuator—a structure that can be used to manipulate the catheter shaft 436, such as torqueing the catheter shaft 436 and/or advancing the catheter shaft 436 relative to a patient's vasculature. That is, the catheter handle 532 may engage the catheter shaft 436 in a manner that allows the catheter handle 532 to be used by a user to move the catheter shaft 436 (e.g., torque and/or advance the catheter shaft 436 relative to the patient's vasculature).

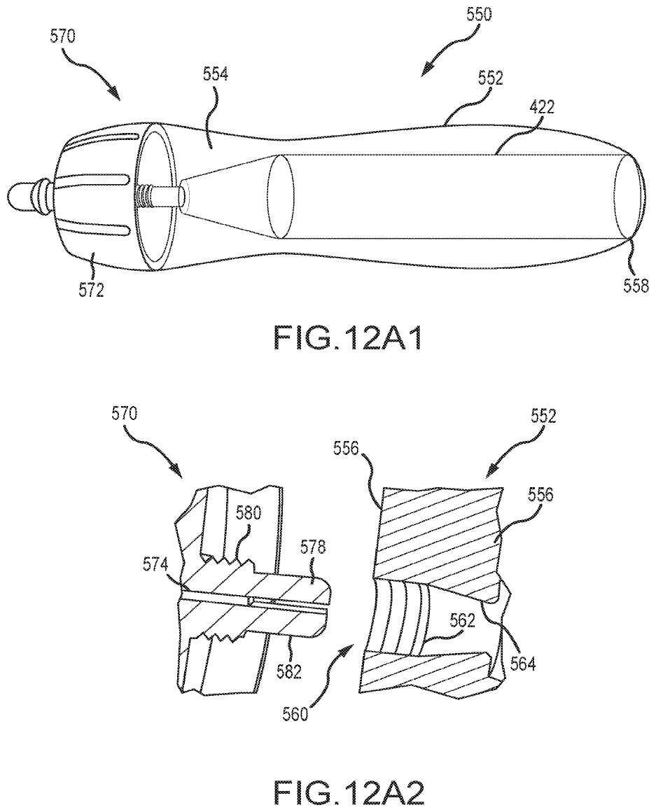

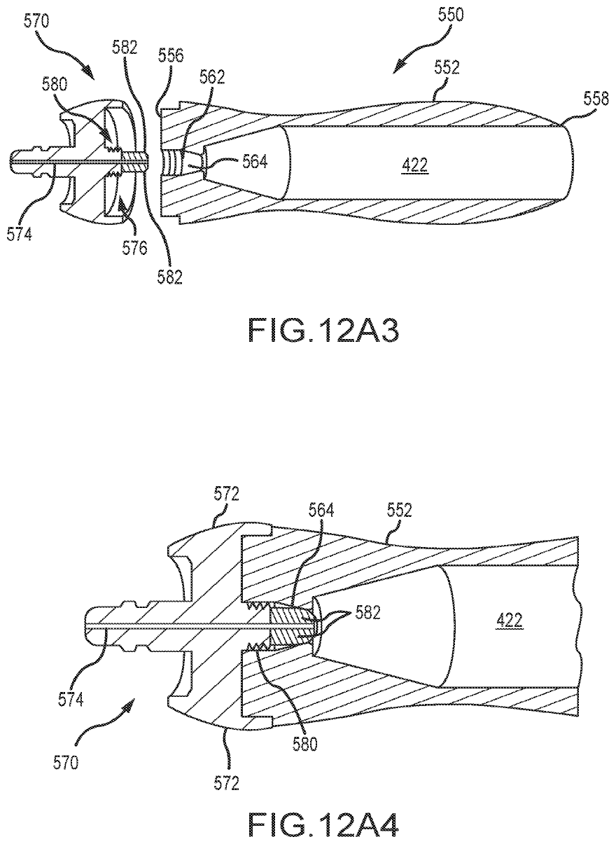

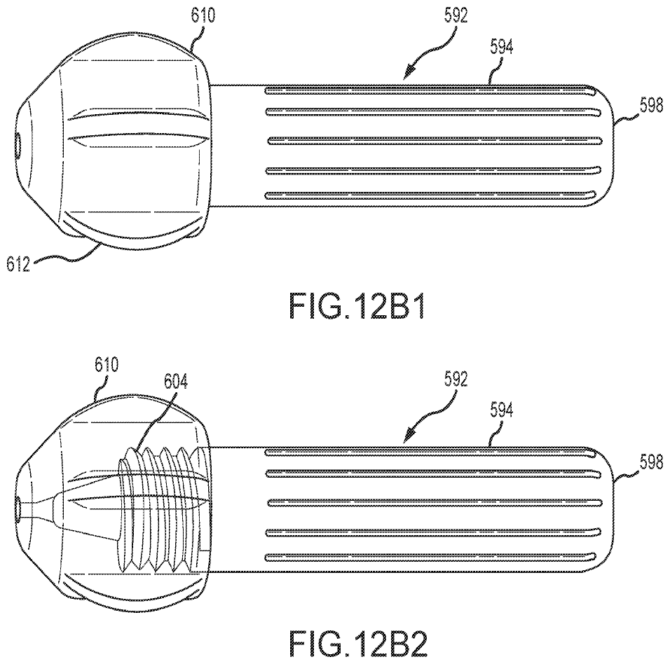

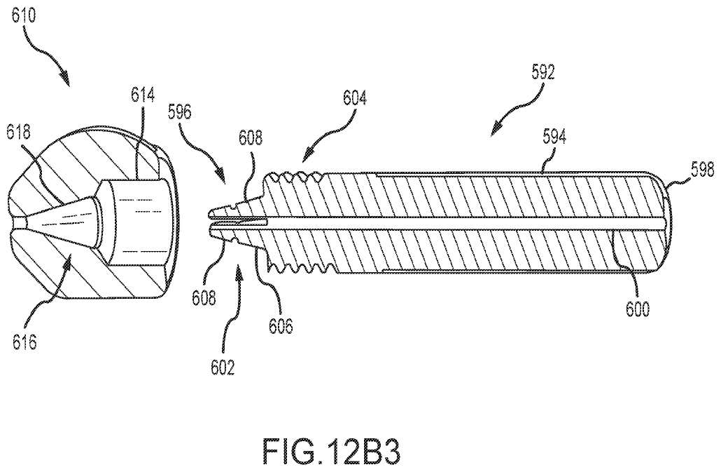

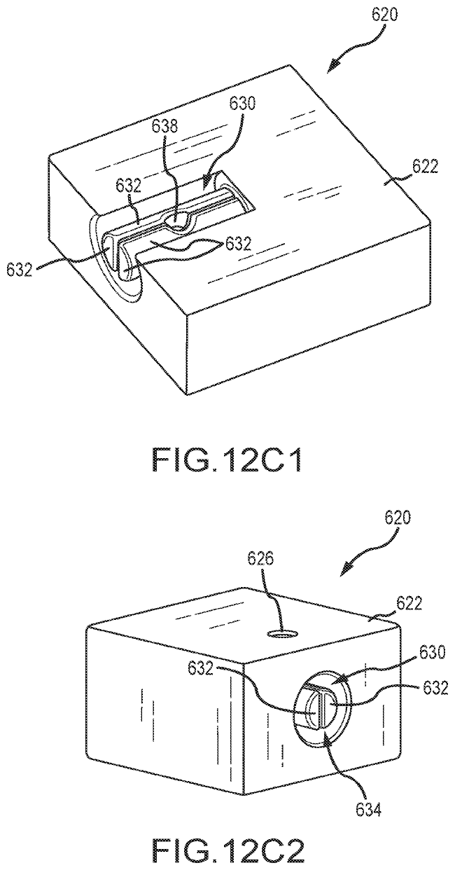

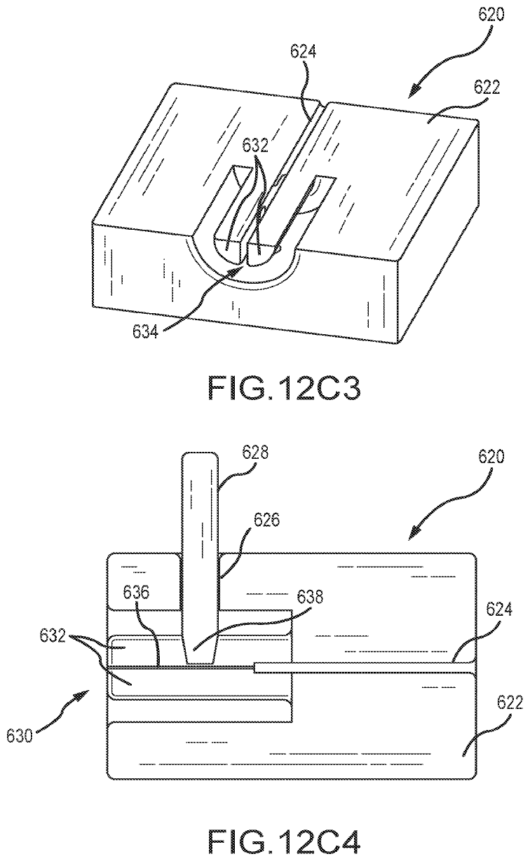

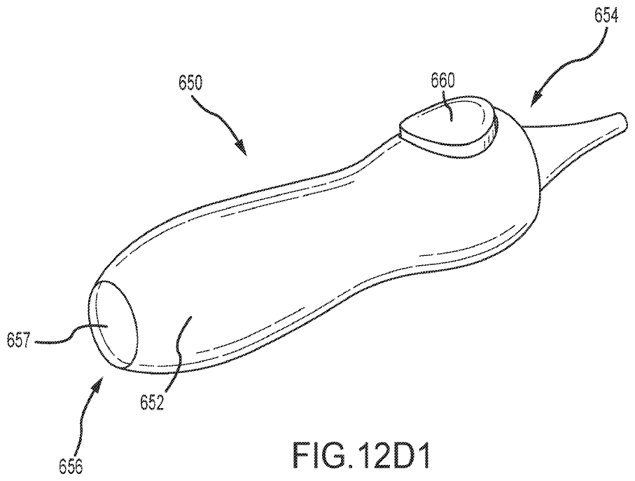

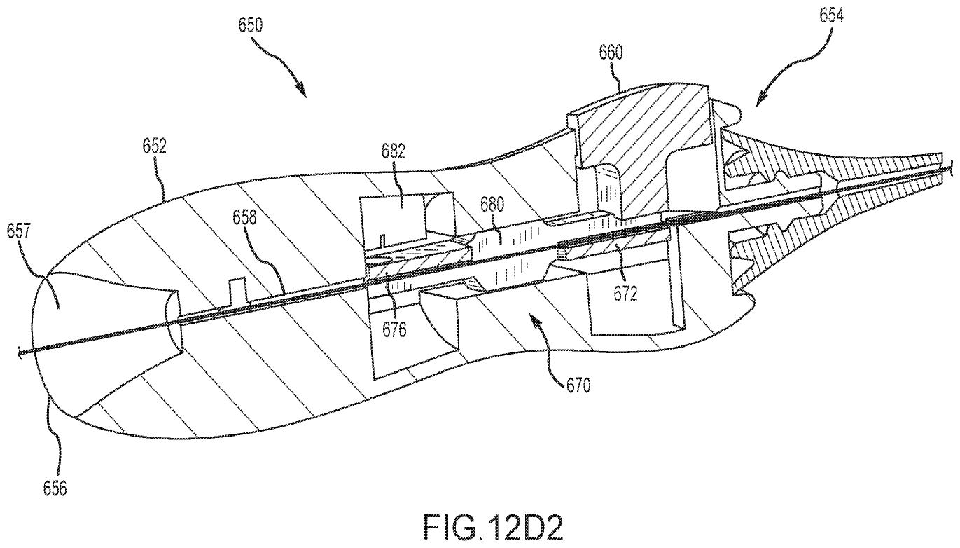

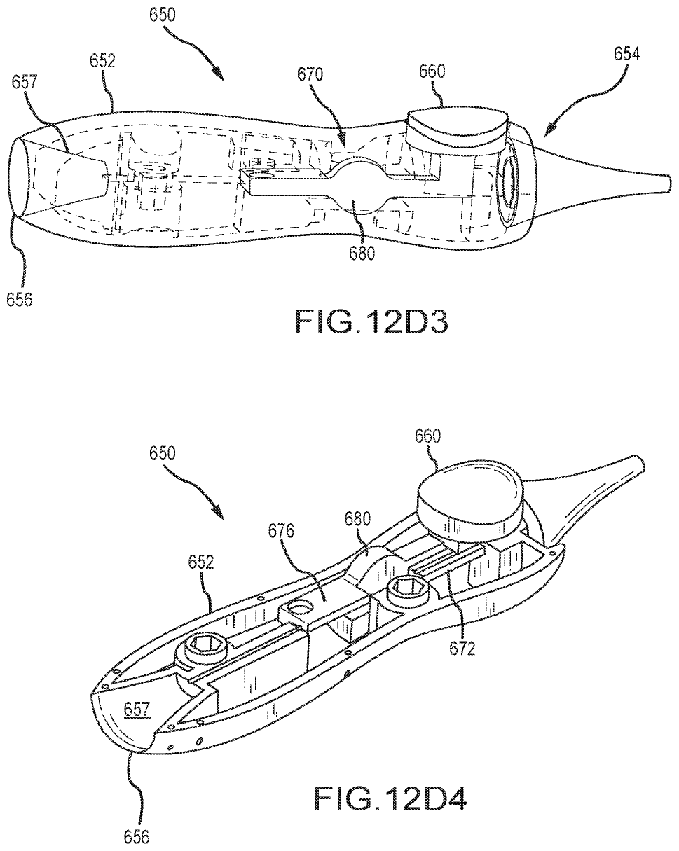

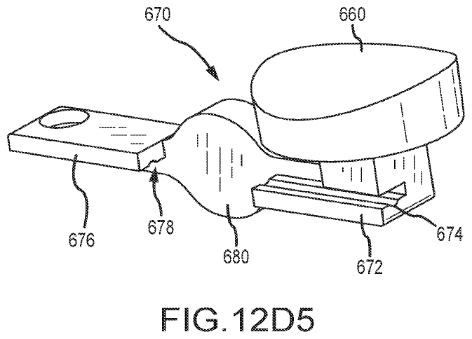

The catheter 530 may include a clamping actuator 540 that may be moved relative to the catheter handle 532 to change a catheter shaft clamp (not shown) of the catheter 530 between a clamped configuration (relative to the catheter shaft 436) and a released configuration (relative to the catheter shaft 436). The clamping actuator 540 is located at/near the distal end 536 of the catheter handle 532 in the illustrated embodiment, and may be rotated relative to the catheter handle 532 to change the corresponding catheter shaft clamp between its clamped configuration and its released configuration. The clamping actuator 540 may be in accordance with the discussion of a clamping actuator 570 discussed below in relation to FIGS. 12A1-12A4. However, any of the various clamping actuators and corresponding catheter shaft clamp addressed herein may be used by the catheter handle 532.

When the clamping actuator 540 is actuated/moved to dispose the corresponding catheter shaft clamp in its clamped configuration, the catheter handle 532 should be maintained in a fixed position relative to the catheter shaft 436 such that the catheter handle 532 may be used to move/manipulate the catheter shaft 436 relative to the patient's vasculature. When the clamping actuator 540 is moved/actuated relative to the catheter handle 532 to dispose the catheter shaft clamp in its released configuration, this allows the catheter handle 532 to be moved along and relative to the catheter shaft 436 to dispose the catheter handle 532 in a desired position relative to the catheter shaft 436 (e.g., to provide a desired working length for the catheter shaft 436). Typically it is the catheter handle 532 that is moved, with the catheter shaft 436 remaining stationary during this adjustment of the noted relative position. As such, the housing 422 should remain in a stationary position at this time (i.e., during movement of the catheter handle 532 relative to the catheter shaft 436). Once the catheter handle 532 is in the desired position along the length of the catheter shaft 436, the clamping actuator 540 may be actuated/moved relative to the catheter handle 532 to dispose the catheter shaft clamp back into its clamped configuration.

In the case of the catheter assembly 480 of FIGS. 11A1-11A4, the proximal end 498 of the catheter handle 492 is disposed adjacent to a distal end 422a of the housing 422 and with the catheter handle 492 being in its proximal-most position relative to the catheter shaft 436. In contrast and for the case of the catheter assembly 520 of FIGS. 11B1-11B3, the housing 422 actually extends into the catheter handle 532 through its proximal end 538 (i.e., the distal end 422a of the housing 422 is disposed within the interior of the catheter handle 532) with the catheter handle 532 being in its proximal-most position relative to the catheter shaft 436. At least part of the housing 422 may extend proximally beyond the proximal end 538 of the catheter handle 532 with the catheter handle 532 being in its proximal-most position relative to the catheter shaft 436 (FIGS. 11B1 and 11B3). In the illustrated embodiment, a majority of the length of the housing 422 is disposed within the catheter handle 532 with the catheter handle 532 being in its proximal-most position relative to the catheter shaft 436 (FIGS. 11B1 and 11B3)—a short proximal end section of the housing 422 does extend proximally beyond the proximal end 538 of the catheter handle 532 with the catheter handle 532 being in its proximal-most position relative to the catheter shaft 436 (FIGS. 11B1 and 11B3).

A variation of the catheter assembly 520, shown in FIGS. 11B1-11B3, is illustrated in FIGS. 11B4-11B5 and is identified by reference numeral 520′. Corresponding components of the catheter assembly 520 (FIGS. 11B1-11B3) and the catheter assembly 520′ (FIG. 11B4-11B5) are identified by a common reference numeral and unless otherwise noted herein to the contrary, the foregoing discussion of these corresponding components remains applicable to the catheter assembly 520′ of FIGS. 11B4-11B5. Those corresponding components that differ in at least some respect are identified by a “single prime” designation in the case of the catheter assembly 520′ of FIGS. 11B4-11B5. The primary difference between the catheter assembly 520 (FIGS. 11B1-11B3) and the catheter assembly 520′ (FIGS. 11B4-11B5) is that more of the housing 422 protrudes proximally from the proximal end 422b in FIGS. 11B4-11B5 compared to the catheter assembly 520 of FIGS. 11B1-11B3, all when the catheter shaft actuator or catheter handle 532′ of the catheter 530′ is in its proximal-most position relative to the catheter shaft 436. FIG. 11B5 illustrates that the catheter handle 532′ may include a receiver 544 (e.g., frustumly-shaped) for receipt of the distal end 422a of the housing 422 (e.g., the inwardly-tapering distal end section of the housing 422) when the catheter handle 532′ is in its proximal-most position relative to the catheter shaft 436. In the illustrated embodiment of FIGS. 11B4-11B5, a majority of the length of the housing 422 is disposed proximally beyond the catheter handle 532′ with the catheter handle 532′ being in its proximal-most position relative to the catheter shaft 436—a short distal end section of the housing 422 extends distally relative to the proximal end 538 of the catheter handle 532 with the catheter handle 532 being in its proximal-most position relative to the catheter shaft 436 (FIGS. 11B4 and 11B5).

For the case of each of the catheter assembly 520 (FIGS. 11B1-11B3) and the catheter assembly 520′ (FIGS. 11B4-11B5), it should be appreciated that the corresponding catheter handle 532/532′ may include a clamping actuator and a corresponding catheter shaft clamp of any appropriate type/configuration, for instance in accordance with the following discussion of FIG. 12 et al. In any case and when the clamping actuator is actuated/moved to dispose the catheter shaft clamp in its clamped configuration, the catheter handle 532/532′ should be maintained in a fixed position relative to the catheter shaft 436 such that the catheter handle 532/532′ may be used to move/manipulate the catheter shaft 436 relative to the patient's vasculature. When the clamping actuator is moved/actuated relative to the catheter handle 532/532′ to dispose the catheter shaft clamp in its released configuration, this allows the catheter handle 532/532′ to be moved along and relative to the catheter shaft 436 to dispose the catheter handle 532/532′ in a desired position relative to the catheter shaft 436 (e.g., to provide a desired working length for the catheter shaft 436). Typically it is the catheter handle 532/.532′ that is moved, with the catheter shaft 436 remaining stationary during this adjustment of the noted relative position. As such, the housing 422 should remain in a stationary position at this time (i.e., during movement of the catheter handle 532/532′ relative to the catheter shaft 436). Once the catheter handle 532/532′ is in the desired position along the length of the catheter shaft 436, the clamping actuator may be actuated/moved relative to the catheter handle 532/532′ to dispose the catheter shaft clamp back into its clamped configuration.

An embodiment of a catheter is illustrated in FIGS. 12A1-12A4 and is identified by reference numeral 550. The catheter 550 includes a catheter handle or a catheter shaft actuator 552 and a catheter shaft such as the catheter shaft 436), and furthermore may utilize the above-noted housing 422 (not shown, but which would at least extend proximally from/relative to the catheter handle 552 for at least some relative positions between the catheter handle 552 and the catheter shaft) or the like. The catheter handle 552 in turn includes a body 554, a distal end 556, and a proximal end 558. The catheter handle 552/body 554 may be in accordance with the catheter handle 492/body 494 discussed above in relation to FIG. 11A1, for instance in relation to the shape/contour of the outer perimeter of the catheter handle 552/body 554.

The catheter handle 552 utilizes/incorporates a clamping actuator 570 (e.g., a rotatable wedge clamp) that may be moved to a first position (e.g., to provide a clamped configuration relative to the catheter shaft, for instance catheter shaft 436) such that the catheter handle 552 may be used to move/manipulate the catheter shaft relative to the patient's vasculature, where this catheter shaft would typically extend at least into the interior of the catheter handle 552 (the catheter shaft could further extend into a housing 422, if utilized). The clamping actuator 570 may be moved to a different second position (e.g., to provide a released configuration relative to the catheter shaft) such that the catheter handle 552 may be advanced along the catheter shaft to a desired position, for instance to provide a desired working length for the catheter shaft. Thereafter, the clamping actuator 570 may be disposed back in its first position (e.g., to provide a clamped configuration relative to the catheter shaft) such that the catheter handle 552 may be used to move/manipulate the catheter shaft relative to the patient's vasculature.

The body 554 of the catheter handle 552 includes a receiver 560 at its distal end 556. This receiver 560 includes internal threads 562 and an internal actuation surface 564. The inner diameter of the actuation surface 564 is progressively reduced proceeding from the internal threads 562 in the direction of the proximal end 558 of the catheter handle 552 (e.g., the actuation surface 564 may be disposed in converging relation to the catheter shaft extending therethrough, in proceeding along its length dimension in the direction of the proximal end 558 of the catheter handle 552). The actuation surface 564 may provide a camming function in relation to an interfacing portion of the clamping actuator 570.

The clamping actuator 570 includes an engagement surface 572 that is disposed on an outer perimeter of the clamping actuator 570 and that may be engaged by a user to dispose the clamping actuator 570 in a position that corresponds with a clamped configuration relative to the catheter shaft (e.g., catheter shaft 436), as well as in a position that corresponds with a released configuration relative to the catheter shaft. In this regard, a catheter shaft lumen or passage 574 extends through an entirety of a length of the clamping actuator 570. The clamping actuator 570 also includes a proximal cavity 576, with a protrusion or projection 578 being disposed within this proximal cavity 576. The catheter shaft lumen 574 extends through this protrusion 578 proceeding along the length dimension of the clamping actuator 570.

The protrusion 578 within the proximal cavity 576 includes a threaded section 580 and at least two clamp segments 582. Each clamp segment 582 is spaced from each adjacent clamp segment 582, and with the clamp segments 582 extending proximally of the threaded section 580. With the catheter shaft (e.g., catheter shaft 436) extending through the catheter shaft lumen 574 of the clamping actuator 570 and into and/or through the catheter handle 552, the protrusion 578 of the clamping actuator 570 may be directed into the receiver 560 of the body 554 of the catheter handle 552 to engage the threaded section 580 of the clamping actuator 570 with the internal threads 562 of the receiver 560. Rotation of the clamping actuator 570 relative to the body 554 of the catheter handle 552 will threadably engage the clamping actuator 570 with the body 554 of the catheter handle 552, and will advance the clamping actuator 570 (relative to the catheter handle 552) in the direction of the proximal end 558 of the catheter handle 552. During this relative motion and with the clamp segments 582 of the clamping actuator 570 engaging the actuation surface 564, the clamp segments 582 will deflect radially inwardly (e.g., via a camming action) to exert a clamping force on the catheter shaft and will dispose the clamp segments 582 in the clamped configuration. The clamping actuator 570 may be rotated in the opposite direction relative to the body 554 of the catheter handle 552 to dispose the clamp segments 582 in the released configuration.

An embodiment of a catheter is illustrated in FIGS. 12B1-12B3 and is identified by reference numeral 590. The catheter 590 includes a catheter handle or a catheter shaft actuator 592 and a catheter shaft such as the catheter shaft 436, and may further utilize the above-noted housing 422 (not shown, but which would at least extend proximally from/relative to the catheter handle 592 for at least some relative positions between the catheter handle 592 and the catheter shaft) or the like. The catheter handle 592 in turn includes a body 594, a distal end 596, and a proximal end 598. The catheter handle 592/body 594 is at least generally cylindrically-shaped in the illustrated embodiment, but could be in accordance with the catheter handle 492/body 494 discussed above in relation to FIG. 11A1, for instance in relation to the shape/contour of the outer perimeter of the catheter handle 592/body 594.

The catheter handle 592 utilizes/incorporates a clamping actuator 610 (e.g., a rotatable cap) that may be moved to a first position (e.g., to provide a clamped configuration relative to a catheter shaft, for instance catheter shaft 436) such that the catheter handle 592 may be used to move/manipulate the catheter shaft relative to the patient's vasculature, where this catheter shaft would typically extend at least into the interior of the catheter handle 592 (the catheter shaft could further extend to a housing 422, if utilized). The clamping actuator 610 may be moved to a different second position (e.g., to provide a released configuration relative to the catheter shaft) such that the catheter handle 592 may be advanced along the catheter shaft to a desired position, for instance to provide a desired working length for the catheter shaft. Thereafter, the clamping actuator 610 may be moved back to the noted first position (e.g., to provide a clamped configuration relative to the catheter shaft) such that the catheter handle 592 may be used to move/manipulate the catheter shaft relative to the patient's vasculature.

A distal end section 602 of the catheter handle 592 includes a threaded section 604 (external threads), along with a protrusion or projection 606 that extends distally of the threaded section 604. The protrusion 606 includes at least two clamp segments 608. Each clamp segment 608 is spaced from each adjacent clamp segment 608. A catheter shaft lumen 600 extends through the entire length of the catheter handle 592, including through the protrusion 606.

The clamping actuator 610 includes an engagement surface 612 that is disposed on an outer perimeter of the clamping actuator 610 and that may be engaged by a user to dispose the clamping actuator 610 in a position that corresponds with a clamped configuration relative to a catheter shaft, as well as in the position that corresponds with a released configuration relative to a catheter shaft. In this regard, a catheter shaft lumen or passage extends through an entirety of a length of the clamping actuator 610.

The clamping actuator 610 includes a proximal section 614. This proximal section 614 includes an internal cavity with internal threads (not shown, but similar to the above-discussed receiver 560 of FIGS. 12A1-12A4). A hollow distal section 616 extends distally of the proximal section 614. The diameter of an inner actuation surface 618 of the distal section 616 (e.g., at least generally in accord with the actuation surface 564 discussed above in relation to FIGS. 12A1-12A4) is progressively reduced proceeding distally from the proximal section 614 of the clamping actuator 610 (e.g., the inner actuation surface 618 of the distal section 616 may be disposed in converging relation to a catheter shaft extending therethrough, in proceeding along its length dimension in the direction of the distal end of the clamping actuator 610). The inner actuation surface 618 of the distal section 616 may provide a camming function in relation to an interfacing portion of the catheter handle 592 (e.g., the protrusion 606).

With a catheter shaft (e.g., catheter shaft 436) extending at least into the catheter shaft lumen 600 of the catheter handle 592, extending distally beyond the catheter handle 592, and through the clamping actuator 610 (such that the catheter shaft extends distally beyond the clamping actuator 610), the protrusion 606 of the catheter handle 592 may be directed into the open end of the proximal section 614 of the clamping actuator to engage the threaded section 604 of the catheter handle 592 with the internal threads of the proximal section 614 of the clamping actuator 610. Rotation of the clamping actuator 610 relative to the body 594 of the catheter handle 592 will threadably engage the clamping actuator 610 with the body 594 of the catheter handle 592, and will advance the clamping actuator 610 (relative to the catheter handle 592) in the direction of the proximal end 598 of the catheter handle 592. During this relative motion and with the clamp segments 608 of the catheter handle 592 engaging the inner actuation surface 618 of the distal section 616, the clamp segments 608 will deflect radially inwardly (e.g., via a camming action) to exert a clamping force on the catheter shaft and will dispose the clamping actuator 610 in its clamped configuration. The clamping actuator 610 may be rotated in the opposite direction relative to the body 594 of the catheter handle 592 to dispose the clamping actuator 570 in its released configuration.

A schematic of an embodiment of a catheter shaft actuator or catheter handle is illustrated in FIGS. 12C1-12C4 and is identified by reference numeral 620. The catheter handle 620 utilizes a clamping actuator 628 and a catheter shaft clamp 630. Otherwise, the catheter handle 620 may be in accordance with any of the various catheter handles addressed herein, and furthermore may be used in combination with a catheter shaft (e.g., catheter shaft 436) and a housing 422.

The catheter handle 620 includes a body 622. A catheter shaft lumen 624 extends through the body 622 of the catheter handle 620, from a distal end of the catheter handle 620 to a proximal end of the catheter handle 620. A clamping actuator passage 626 extends from a location on an exterior of the body 622 of the catheter handle 620, and into the body 622 in the direction of the catheter shaft clamp 630. The clamping actuator 628 is movably disposed within the clamping actuator passage 626, and is accessible on an exterior of the body 622 of the catheter handle 620 (e.g., the clamping actuator 628 may protrude beyond an outer perimeter of the catheter handle 620). One or more biasing members of any appropriate type/configuration (not shown) may bias the clamping actuator 628 away from the catheter shaft clamp 630.

The catheter shaft clamp 630 includes a pair of clamp segments 632 that are separated by a slot 634. The width of the slot 634 is less than the outer diameter of a portion of the catheter shaft (e.g., catheter shaft 436) that passes through the catheter shaft clamp 630. The surfaces of the clamp segments 632 that face one another define a perimeter boundary of a portion of a catheter shaft lumen 636 to accommodate the passage of catheter shaft through the catheter shaft clamp 630. Each clamp segment 632 includes what may be characterized as an actuation surface 638 that is disposed in converging relation to the corresponding catheter shaft lumen 636, proceeding in the direction of the catheter shaft lumen 636 (the actuator surface 638 being “behind” the clamping actuator 628 in the view shown in FIG. 12C4). The actuation surfaces 638 of the clamp segments 632 may be characterized as defining a drafted wedge slot that converges proceeding in a direction that is away from the aligned clamping actuator passage 626.

When a catheter shaft (e.g., catheter shaft 436) is loaded in the catheter handle 620 and is directed through the catheter shaft clamp 630, the clamp segments 632 will clamp against the catheter shaft due to an interference fit between the clamp segments 632 and the catheter shaft (e.g., a clamped configuration for the catheter shaft clamp 630). At this time, the catheter handle 620 may be used to move/manipulate the catheter shaft relative to the patient's vasculature. When it is desired to reposition the catheter handle 620 along the catheter shaft, for instance to provide a desired working length for the catheter shaft, the clamping actuator 628 may be advanced in the direction of the catheter shaft clamp 630 to engage the actuation surfaces 638 and the clamp segments 632. Continued movement of the clamping actuator 628 relative to the catheter shaft clamp 630, and while the clamping actuator 628 is engaged with the actuation surfaces 638 of the clamp segments 632, will increase the spacing between the clamp segments 632 to dispose the catheter shaft clamp 630 in its released configuration (e.g., spread apart the clamp segments 632). With the catheter shaft clamp 630 being in its released configuration, and that may require continued exertion of an actuating force on the clamping actuator 628, the catheter handle 620 may be moved relative to and along the catheter shaft to dispose the catheter handle 620 in a desired position (e.g., to provide a desired working length for the catheter shaft). Thereafter, the clamping actuator 628 may be released, which will move the clamping actuator 628 away from the catheter shaft clamp 630, and which in turn will allow the clamp segments 632 to return to their clamped configuration relative to the catheter shaft (via at least a reduction of the force exerted by the clamping actuator 628 on the actuation surfaces 638 of the clamp segments 632). Thereafter, the catheter handle 620 may be used to move/manipulate the catheter shaft relative to the patient's vasculature.

An embodiment of a catheter handle or a catheter shaft actuator is illustrated in FIGS. 12D1-12D5 and is identified by reference numeral 650. The catheter handle 650 includes a body 652. A catheter shaft lumen 658 extends through the body 652 of the catheter handle 650, from a distal end 654 of the catheter handle 650 to a proximal end 656 of the catheter handle 650. A catheter shaft 436 may be disposed within this catheter shaft lumen 658, with the catheter shaft 436 extending distally beyond the distal end 654 of the catheter handle 650, and typically with the catheter shaft 436 extending proximally beyond the proximal end 656 of the catheter handle 650 (e.g., extending to a housing 422). In this regard, the proximal end 656 of the catheter handle 650 may include a receiver 657 to accommodate receipt of a distal end section of the housing 422 (e.g., in accord with FIGS. 11B4 and 11B5, discussed above).

The catheter handle 650 includes/utilizes a clamping actuator 660 for disposing a catheter shaft clamp 670 in a released configuration relative to the catheter shaft 436 (to accommodate relative movement between the catheter handle 650 and the catheter shaft 436, such that the catheter handle 650 may be moved to a desired position along/relative to the catheter shaft 436 (e.g., to provide a desired working length for the catheter shaft 436). Typically it is the catheter handle 650 that is moved, with the catheter shaft 436 remaining stationary during this adjustment of the noted relative position. As such, a housing 422 (to which the catheter shaft 436 may extend, and located proximally of the catheter handle 650), should remain in a stationary position at this time (i.e., during movement of the catheter handle 650 relative to the catheter shaft 436). The catheter shaft clamp 670 is also disposable in a clamped configuration relative to the catheter shaft 436, which should maintain the catheter handle 650 in a fixed position relative to the catheter shaft 436 such that the catheter handle 650 may be used to move/manipulate the catheter shaft 436 relative to the patient's vasculature. The catheter shaft clamp 660 may be biased to its clamped configuration.

The catheter shaft clamp 670 includes what may be characterized as distal clamping section 672, a proximal clamping section 676, and a rotary body 680, with the rotary body 680 being located between the distal clamping section 672 and the proximal clamping section 676 along/relative to the length dimension of the catheter handle 650. The distal clamping section 672 includes a groove 674 (on an upwardly-facing surface of the distal clamping section 672 in the view presented in FIG. 12D5) for receipt of a corresponding portion of the catheter shaft 436, while the proximal clamping section 676 similarly includes a groove 678 (but on the downwardly-facing surface of the distal clamping section 672 in the view presented in FIG. 12D5) for receipt of a corresponding portion of the catheter shaft 436. One or more biasing members 682 are disposed on one side of the rotary body 680, while a clamping actuator 660 is disposed on the opposite side of the rotary body 680 (e.g., the catheter shaft clamp 670 is at least generally of a teeter-totter type configuration).

In the views illustrated in FIGS. 12D2 and 12D5, the biasing member(s) 682 exerts a downwardly-directed force on an upper surface the proximal clamping section 676, which causes the proximal clamping section 676 to exert a downwardly-directed clamping force on the catheter shaft 436, and simultaneously causes the distal clamping section 672 to exert an upwardly-directed clamping force on the catheter shaft 436, all of which disposes the catheter shaft clamp 670 in its clamped configuration. The noted biasing force of the biasing member(s) 682 induces a counterclockwise movement of the catheter shaft clamp 670 in the view presented in FIG. 12D2. These oppositely-directed and spaced clamping forces exerted on the catheter shaft 436 provide the clamped configuration, as the catheter shaft 436 is “vertically” restrained by being positioned within catheter shaft lumen 658 on opposite sides of the catheter shaft clamp 670. However, there is an open space below the proximal clamping section 676 and an open space above the distal clamping section 672. Again, the clamped configuration for the catheter shaft clamp 670 should maintain the catheter handle 650 in a fixed position relative to the catheter shaft 436 such that the catheter handle 650 may be used to move/manipulate the catheter shaft 436 relative to the patient's vasculature.

The clamping actuator 660 is fixed to the catheter shaft clamp 670 in the illustrated embodiment, although such need not be the case. In the view presented in FIGS. 12D2 and 12D5, pushing down on the clamping actuator 660 moves the distal clamping section 672 in a downward direction and simultaneously moves the proximal clamping section 676 in an upward direction (these movements being opposed by the biasing member(s) 682), all of which disposes the catheter shaft clamp 670 in its released configuration. Again, the released configuration accommodates relative movement between the catheter handle 650 and the catheter shaft 436, such that the catheter handle 650 may be moved to a desired position along the catheter shaft 436 (e.g., to provide a desired working length for the catheter shaft 436). Once the catheter handle 650 is in the desired position along the length of the catheter shaft 436, the clamping actuator 660 may be released. The biasing member(s) 682 should then automatically return the catheter shaft clamp 670 to its clamped configuration, and will also automatically move the clamping actuator 660 away from its position that provides for a released configuration for the catheter shaft clamp 670.

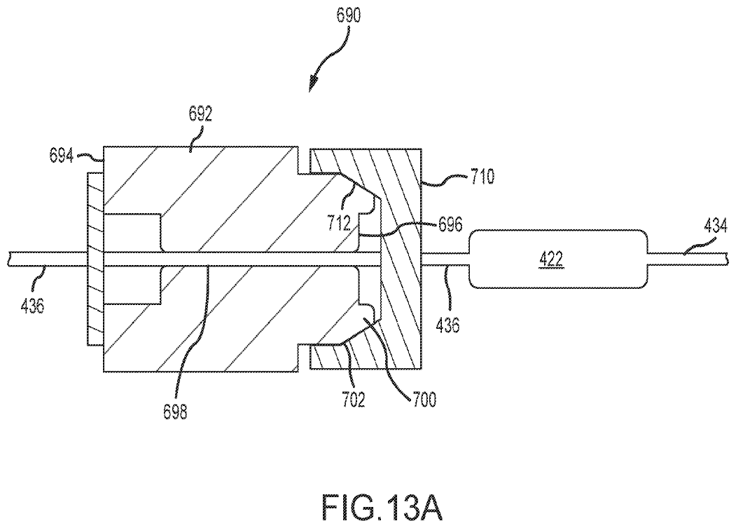

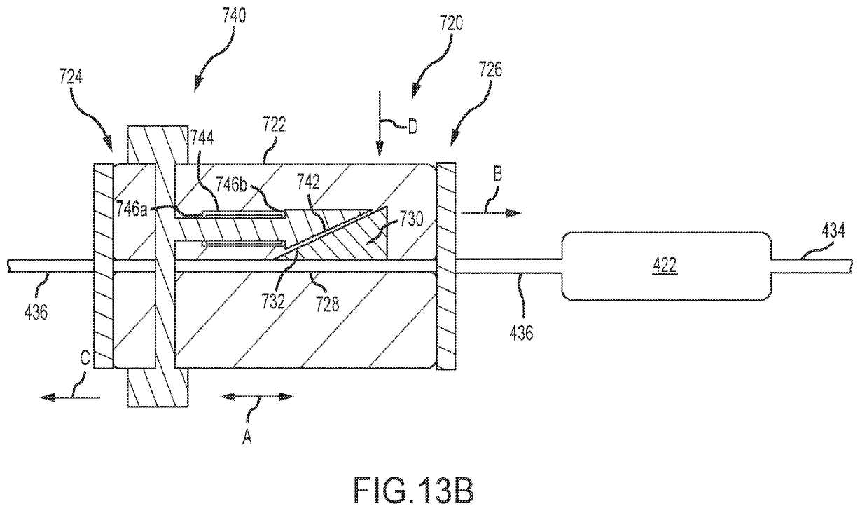

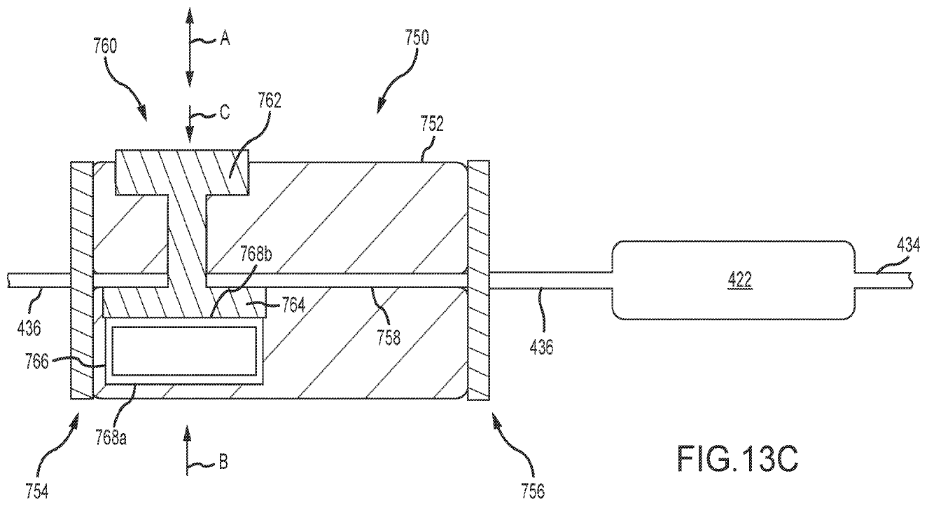

An embodiment of a catheter handle or a catheter shaft actuator is schematically illustrated in FIG. 13A, is identified by reference numeral 690, and may be used in conjunction with the above-noted housing 422 or the like. The catheter handle 690 includes a body 692. A catheter shaft lumen 698 extends through the body 692 of the catheter handle 690, from a distal end 694 of the catheter handle 690 to a proximal end 696 of the catheter handle 690. A catheter shaft 436 may be disposed within this catheter shaft lumen 698, with the catheter shaft 436 extending distally beyond the distal end 694 of the catheter handle 690, and typically with the catheter shaft 436 extending proximally beyond the proximal end 696 of the catheter handle 690 (e.g., to/into the housing 422, where it may be maintained in a fixed position relation to the housing 422).

The catheter handle 690 includes/utilizes a clamping actuator 710 (e.g., a collet or collet-type mechanism having an actuating surface 712 (e.g., a cam). A distal end section of the illustrated catheter handle 690 includes a catheter shaft clamp 700 having an actuated surface 702 (e.g., a follower). The catheter shaft lumen 698 extends through the clamp 700. The clamping actuator 710 and the catheter handle 690 may be rotatably interconnected (e.g., via a threaded connection). Rotation of the clamping actuator 710 relative to the catheter handle 690 in one direction will dispose the actuating surface 712 of the clamping actuator 710 in interfacing relation with the actuated surface 702 of the clamp 700. Continued rotation will cause the actuated surface 702 of the catheter shaft clamp 700 to cam off of the actuating surface 712 of the clamping actuator 710 and force the catheter shaft clamp 700 against the catheter shaft 436 (e.g., by a radially-inward deflection/movement of the catheter shaft clamp 700 and relative to the catheter handle 690). Disposing the catheter shaft clamp 700 in this clamped configuration relative to the catheter shaft 436 should maintain the catheter handle 690 in a fixed position relative to the catheter shaft 436 such that the catheter handle 690 may be used to move/manipulate the catheter shaft 436 relative to the patient's vasculature. Rotation of the clamping actuator 710 relative to the catheter handle 690 in an opposite direction to that described above will dispose the catheter shaft clamp 700 in a released configuration relative to the catheter shaft 436 (e.g., by at least reducing an engagement between the actuating surface 712 of the clamping actuator 710 and the actuated surface 702 of the catheter shaft clamp 700, including disengaging the actuating surface 712 from the actuated surface 702). As such, the catheter shaft clamp 700 should return toward a neutral position (e.g., via an elasticity of the catheter shaft clamp 700) where the clamping force being exerted by the catheter shaft clamp 700 on the catheter shaft 436 is reduced or eliminated. Disposing the catheter shaft clamp 700 in its released configuration accommodates relative movement between the catheter handle 690 and the catheter shaft 436, such that the catheter handle 650 may be moved to a desired position along the catheter shaft 436 (e.g., to provide a desired working length for the catheter shaft 436). Typically it is the catheter handle 690 that is moved, with the catheter shaft 436 remaining stationary during this adjustment of the noted relative position. As such, the housing 422 should remain in a stationary position at this time as well (i.e., during movement of the catheter handle 690 relative to the catheter shaft 436).

An embodiment of a catheter handle or a catheter shaft actuator is schematically illustrated in FIG. 13B, is identified by reference numeral 720, and may be used in conjunction with the above-noted housing 422 or the like. The catheter handle 720 includes a body 722. A catheter shaft lumen 728 extends through the body 722 of the catheter handle 720, from a distal end 724 of the catheter handle 720 to a proximal end 726 of the catheter handle 720. A catheter shaft 436 may be disposed within this catheter shaft lumen 728, with the catheter shaft 436 extending distally beyond the distal end 724 of the catheter handle 720, and typically with the catheter shaft 436 extending proximally beyond the proximal end 726 of the catheter handle 720 (e.g., to the housing 422).

The catheter handle 720 includes/utilizes a catheter shaft clamp 730, along with a clamping actuator 740. The catheter shaft clamp 730 is movable in a dimension represented by the double-headed arrow A in FIG. 13B, and includes an actuated surface 732 (e.g., the catheter shaft clamp 730 may be referred to as a follower in a cam/follower arrangement). The clamping actuator 740 includes an actuating surface 742 (e.g., the clamping actuator 740 may be referred to as a cam in a cam/follower arrangement). One or more biasing members 744 of any appropriate type/configuration (e.g., a coil spring) may exert a biasing force on the clamping actuator 740 in the direction indicated by the arrow B in FIG. 13B. The biasing member(s) 744 includes a fixed end 746a (fixed relative to the clamping actuator 740 and the catheter handle 720) and a movable end 746b (movable relative to the clamping actuator 740 and the catheter handle 720) that engages and biases the clamping actuator 740 in the direction of the arrow B (relative to the catheter handle 720).

Without exerting a release force on the clamping actuator 740 in the direction of the arrow C in FIG. 13B, the biasing member 744 will exert a biasing force on the clamping actuator 740 (in the direction of the arrow B, and of a constant magnitude), which will dispose the actuating surface 742 of the clamping actuator 740 in interfacing relation with the actuated surface 732 of the catheter shaft clamp 730, which in turn will move the catheter shaft clamp 730 (e.g., axially) in the direction of the arrow D and into engagement with the catheter shaft 436 to dispose the catheter shaft clamp 730 in a clamped configuration relative to the catheter shaft 436. Disposing the catheter shaft clamp 730 in this clamped configuration relative to the catheter shaft 436 should maintain the catheter handle 720 in a fixed position relative to the catheter shaft 436 such that the catheter handle 720 may be used to move/manipulate the catheter shaft 436 relative to the patient's vasculature. To dispose the catheter shaft clamp 730 in its released configuration, a release force is exerted on the clamping actuator 740 in the direction of the arrow C in FIG. 13B and that will move the clamping actuator 740 relative to the catheter handle 720 in the direction indicated by the arrow C. This motion of the clamping actuator 740 will compress the biasing member(s) 744 and will at least reduce the engagement between the actuating surface 742 of the clamping actuator 740 and the actuated surface 732 of the catheter shaft clamp 730, including disengaging the actuating surface 742 from the actuated surface 732 (thereby reducing or eliminating the clamping force being exerted by the catheter shaft clamp 740 on the catheter shaft 436), all of which disposes the catheter shaft clamp 730 in its released configuration. Disposing the catheter shaft clamp 730 in its released configuration accommodates relative movement between the catheter handle 720 and the catheter shaft 436, such that the catheter handle 720 may be moved to a desired position along the catheter shaft 436 (e.g., to provide a desired working length for the catheter shaft 436). Typically it is the catheter handle 720 that is moved, with the catheter shaft 436 remaining stationary during this adjustment of the noted relative position. As such, the housing 422 should remain in a stationary position at this time (i.e., during movement of the catheter handle 720 relative to the catheter shaft 436).

An embodiment of a catheter handle or a catheter shaft actuator is schematically illustrated in FIG. 13C, is identified by reference numeral 750, and may be used in conjunction with the above-noted housing 422 or the like. The catheter handle 750 includes a body 752. A catheter shaft lumen 758 extends through the body 752 of the catheter handle 720, from a distal end 754 of the catheter handle 750 to a proximal end 756 of the catheter handle 750. A catheter shaft 436 may be disposed within this catheter shaft lumen 758, with the catheter shaft 436 extending distally beyond the distal end 754 of the catheter handle 750, and typically with the catheter shaft 436 extending proximally beyond the proximal end 756 of the catheter handle 750 (e.g., to the housing 422).

The catheter handle 750 includes/utilizes a clamping actuator 760. The clamping actuator 760 is movable in a dimension represented by the double-headed arrow A in FIG. 13C. The clamping actuator 760 includes an actuation section 762 (e.g., an actuator) and a clamping section 764 (e.g., a catheter shaft clamp). One or more biasing members 766 of any appropriate type/configuration (e.g., a coil spring) may exert a biasing force on the clamping actuator 740 in the direction indicated by the arrow B in FIG. 13C. The biasing member 766 includes a fixed end 768a (fixed relative to the catheter handle 750) and a movable end 768b (movable relative to the catheter handle 750) that engages the clamping section 764 and moves with the clamping actuator 760 (relative to the body 752 of the catheter handle 750).

Without exerting a release force on the clamping actuator 740 in the direction of the arrow C in FIG. 13C, the biasing member(s) 766 will exert a biasing force on the clamping section 764 of the clamping actuator 760 (in the direction of the arrow B), which will dispose the clamping section 764 of the clamping actuator 760 in interfacing relation with the catheter shaft 436 and exert a clamping force thereon to dispose the catheter shaft clamp 730 in a clamped configuration. Disposing the clamping section 764 in this clamped configuration relative to the catheter shaft 436 should maintain the catheter handle 750 in a fixed position relative to the catheter shaft 436 such that the catheter handle 750 may be used to move/manipulate the catheter shaft 436 relative to the patient's vasculature. To dispose the clamping section 764 in its released configuration, a release force is exerted on the actuation section 762 of the clamping actuator 760 in the direction of the arrow C in FIG. 13C and that will move the clamping actuator 760 relative to the catheter handle 750 in the direction indicated by the arrow C. This motion of the clamping actuator 760 will compress the biasing member(s) 766 and will at least reduce the engagement between the clamping section 764 of the clamping actuator 760 and the catheter shaft 436, including disengaging the clamping section 764 of the clamping actuator 760 from the catheter shaft 436 (e.g., reduces/eliminates the clamping force exerted by the clamping section 764 on the catheter shaft 436). In any case, disposing the clamping actuator 760 in its released configuration accommodates relative movement between the catheter handle 750 and the catheter shaft 436, such that the catheter handle 750 may be moved to a desired position along the catheter shaft 436 (e.g., to provide a desired working length for the catheter shaft 436). Typically it is the catheter handle 750 that is moved, with the catheter shaft 436 remaining stationary during this adjustment of the noted relative position. As such, the housing 422 should remain in a stationary position at this time (i.e., during movement of the catheter handle 750 relative to the catheter shaft 436).

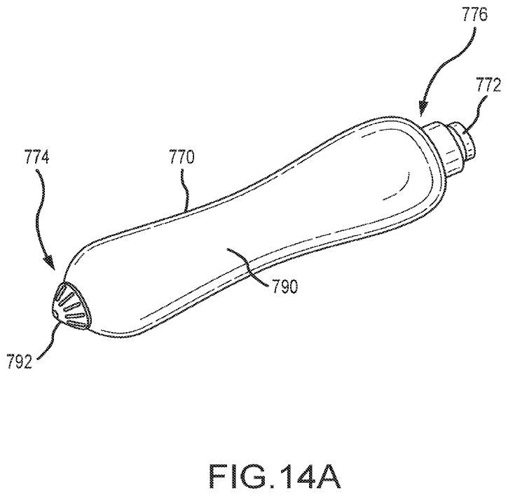

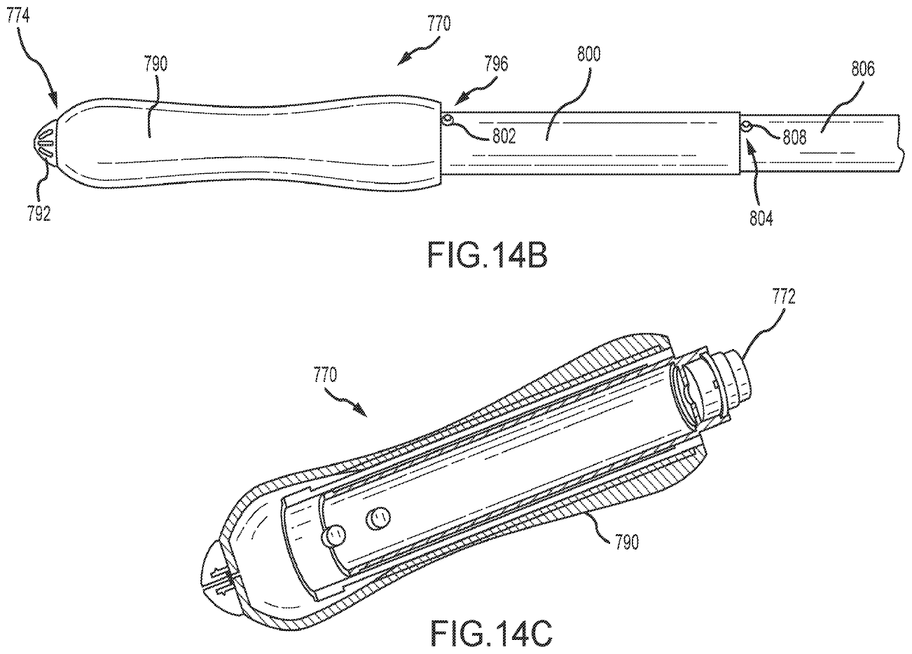

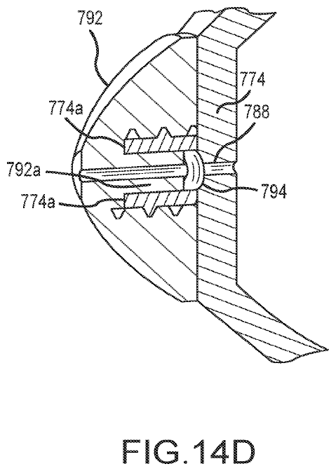

An embodiment of a catheter handle is illustrated in FIGS. 14A-14D, is identified by reference numeral 770, and may be used in conjunction with the above-noted housing 422 or the like. The catheter handle 770 utilizes a telescoping configuration, and includes a primary handle section 790, a first handle extension 800, and an optional second handle extension 806. Any number of handle extensions may be used by the catheter handle 770. A distal end 774 of the catheter handle 770 is incorporated by the primary handle section 790, while a proximal end 776 of the catheter handle 770 is defined by the second handle extension 806 (the proximal-most extension used by the catheter handle 770). A connector 772 may be secured to the proximal-most extension (the second extension 806 in the illustrated embodiment) and may be located at least generally at the proximal end 776 of the catheter handle 770. One or more wires, a cable (e.g., generator cable 434), or the like may extend proximally from the connector 772.

The catheter handle 770 includes/utilizes a clamping actuator 792 and a clamping ring 794. A catheter shaft (e.g. catheter shaft 436) extends through the clamping actuator 792 and the clamping ring 794, and into/through a catheter shaft lumen 788 that extends at least partially along the length dimension of the primary handle section 790. In any case, the catheter shaft would extend through the catheter handle 770 and at least to the connector 772.

The clamping actuator 792 is disposed at the distal end 774 of the catheter handle 770 and is rotatably interconnected with the catheter handle 770 (e.g., via a threaded engagement between the clamping actuator 792 and the catheter handle 770). The catheter shaft 436 extends through this clamping actuator 792 and through the clamping ring 794. The clamping ring 794 is disposed within an interior cavity of a distal section 744a and is seated against a corresponding portion of the distal end 644 of the catheter handle 770 (and through which the catheter shaft 436 extends). The clamping actuator 792 includes a protrusion 992a that extends within the interior cavity of the distal section 744a.

The clamping actuator 792 may be moved relative to the catheter handle 770 to change clamping ring 794 of the catheter handle 770 between a clamped configuration (relative to the catheter shaft) and a released configuration (relative to the catheter shaft). The clamping actuator 792 is located at/near the distal end 774 of the catheter handle 842 in the illustrated embodiment, and may be rotated relative to the catheter handle 770 to change the clamping ring 794 between its clamped configuration and its released configuration.

Rotation of the clamping actuator 792 relative to the catheter handle 770 in one relative direction will compress the clamping ring 794 between the distal end 774 of the catheter handle 770 and the protrusion 792a of the clamping actuator 792 (the protrusion 792a of the clamping actuator 792 moving toward the proximal end 776 of the catheter handle 770), which in turn will dispose the clamping ring 794 in its clamped configuration relative to the catheter shaft. When the clamping actuator 792 is rotated in the noted manner to dispose the clamping ring 794 in its clamped configuration, the catheter handle 770 should be maintained in a fixed position relative to the catheter shaft such that the catheter handle 770 may be used to move/manipulate the catheter shaft relative to the patient's vasculature.

Rotation of the clamping actuator 792 relative to the catheter handle 770 in the opposite relative direction to that described above will allow the clamping ring 794 to expand (the protrusion 792a of the clamping actuator 792 now moving away from the proximal end 776 of the catheter handle 770), which in turn will dispose the clamping ring 794 in its released configuration relative to the catheter shaft. The released configuration allows the catheter handle 770 to be moved along and relative to the catheter shaft to dispose the catheter handle 842 in a desired position relative to the catheter shaft 436 (e.g., to provide a desired working length for the catheter shaft 436). This is by a “telescoping” of the catheter handle 770 in a manner that is addressed below, typically by moving the distal end 774 of the catheter handle 770. Thereafter and with the distal end 774 of the catheter handle 770 being in the desired position along the length of the catheter shaft, the clamping actuator 792 may be rotated relative to the catheter handle 770 to dispose the clamping ring 794 back into its clamped configuration.

The collapsed or compressed configuration for the catheter handle 770 is shown in FIGS. 14A and 14C. A proximal end section of the handle extension that incorporates the connector 772 (the second handle extension 806 in the illustrated embodiment) may extend proximally from the proximal end of the primary handle section 790. In any case, the catheter handle 770 may be configured to detachably retain each of its handle extensions (e.g., handle extensions 800, 806) relative to the primary handle section 790 and in the configuration shown in FIGS. 14A and 14C. This fixed retention of the relative positions of the primary handle section 790 and each of its extensions may be realized in any appropriate manner.

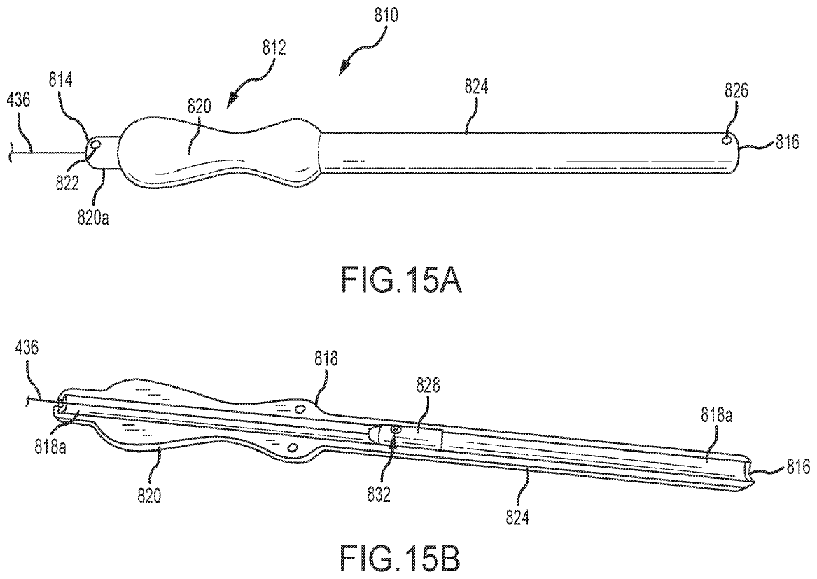

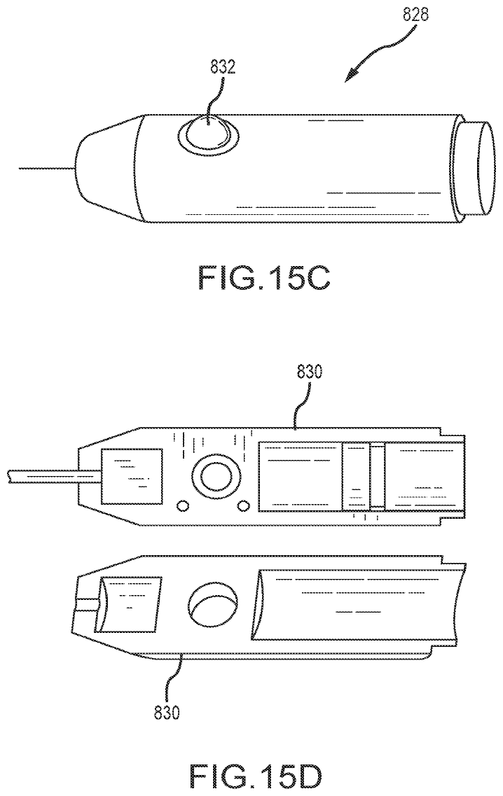

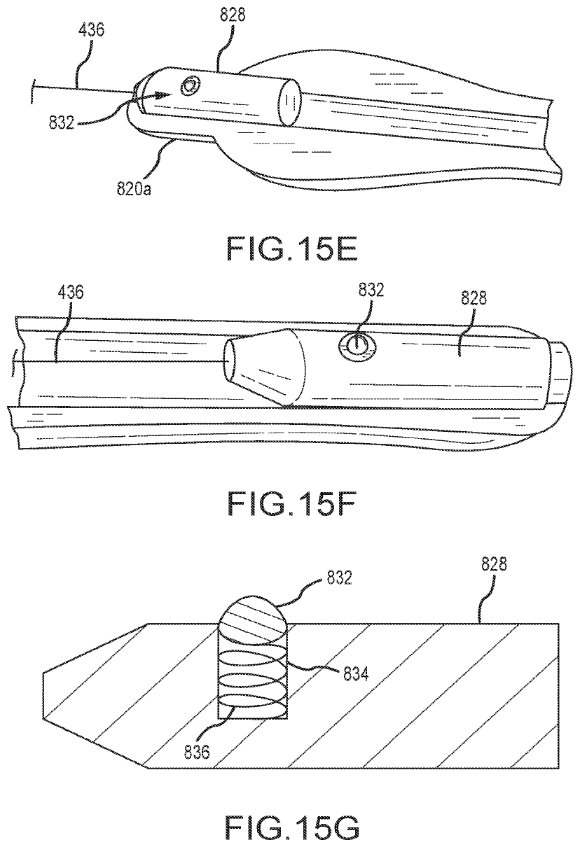

A primary detent 796 (e.g., spring-loaded to an extended position) is associated with the primary handle section 790. The first handle extension 800 includes one or more holes 802 that may be spaced along the length dimension of the first extension 800. The primary handle section 790 may be moved distally relative to the first handle extension 800 (e.g., along a linear or axial path, with the above-noted clamping ring 794 being in its released configuration relative to the catheter shaft, and with the primary detent 796 having been sufficiently depressed), and also relative to the catheter shaft, to a position where the primary detent 796 is aligned with a hole 802 in the first handle extension 800. With the primary detent 796 extending through a hole 802 of the first extension 800 (e.g., by a spring bias or the like exerted on the primary detent 796), the position of the first handle extension 800 relative to the primary handle section 790 may be fixed (e.g., in accord with FIG. 15G, discussed below). This extension of the catheter handle 770 reduces the working length of the catheter shaft (as the catheter shaft again is fixed relative to the connector 772). Stated another way, this telescoping of the catheter handle 770 reduces the spacing between the distal end 774 of the catheter handle 770 and a distal end of the catheter shaft (and similarly reduces the spacing between the distal end 774 and the vasculature access to a patient if the catheter shaft has been directed into the vasculature of the patient). Disposing the primary detent 796 in a more distally-positioned hole 802 of the first extension 800 would of course further reduce the working length of the catheter shaft.

An additional reduction of the working length of the catheter shaft may be realized by collectively telescoping the primary handle section 790 and the first handle extension 800 relative to both the second handle extension 806 and the catheter shaft (e.g., the primary handle section 790 and the first handle extension 800 may be collectively advanced—the primary handle section 790 and the first handle extension 800 being maintained in a fixed position relative to one another at this time—in a distal direction relative to the second handle extension 806 and the catheter shaft). In this regard, the first handle extension 800 includes a first detent 804 (e.g., spring-loaded to an extended position). The second handle extension 806 includes one or more holes 808 that may be spaced along the length dimension of the second handle extension 806. The primary handle section 790 and the first handle extension 800 may be collectively moved distally relative to the second handle extension 806 (e.g., along a linear or axial path, with the above-noted clamping ring 794 being in its released configuration relative to the catheter shaft, and with the first detent 804 having been sufficiently depressed), and also relative to the catheter shaft, to a position where the first detent 804 is aligned with a hole 808 in the second handle extension 806. With the first detent 804 extending through a hole 808 of the second handle extension 806 (e.g., by a spring bias or the like exerted on the first detent 796), the position of the second handle extension 806 relative to first handle extension 800 (as well as relative to the primary handle section 790) may be fixed. This additional extension of the catheter handle 770 further reduces the working length of the catheter shaft. Stated another way, this telescoping of the catheter handle 770 reduces the spacing between the distal end 774 of the catheter handle 770 and a distal end of the catheter shaft (and similarly reduces the spacing between the distal end 774 and the vasculature access to a patient if the catheter shaft has been directed into the vasculature of the patient). Disposing the first detent 804 in a more distally-positioned hole 808 of the second handle extension 806 would of course further reduce the working length of the catheter shaft.

An increase in the working length of the catheter shaft may be realized by at least partially collapsing a previously telescoped catheter handle 770. For instance, the clamping ring 794 may be disposed in its released configuration and: 1) the first detent 804 may be compressed and the second extension 806 may be advanced within the first handle extension 800 to align the first detent 804 with a more proximally-positioned hole 808 of the second handle extension 806 (at which time the first detent 804 may be released so as to protrude through the aligned hole 808); and/or 2) the primary detent 796 may be compressed and the first handle extension 806 may be advanced within the primary handle section 790 to align the primary detent 794 with a more proximally-positioned hole 802 of the first handle extension 800 (at which time the primary detent 796 may be released so as to protrude through the aligned hole 802).

An embodiment of a catheter is illustrated in FIGS. 15A-15G is identified by reference numeral 810. The catheter 810 may be characterized as including a catheter handle 812 and a catheter shaft, such as the above-noted catheter shaft 436. The catheter handle 812 includes a distal end 814 and a proximal end 816 that are spaced along a length dimension of the catheter handle 812, as well as a two or more body sections 818 that may be appropriately interconnected with one another (e.g., joined together; disposable in fixed relation to one another) in any appropriate manner. A catheter shaft clamp (e.g., in accord with any of the various embodiments described herein) could also be utilized by the catheter 810.

There are three primary portions of the catheter handle 812—the primary handle section 820, a distal extension 820a (that includes the noted distal end 814 in the illustrated embodiment), and a proximal handle extension 824 (that includes the noted proximal end 816 in the illustrated embodiment). The proximal handle extension 824 extends proximally from the primary handle section 820, while the distal extension 820a extends distally from the primary handle section 820 in the illustrated embodiment. Both the distal extension 820a and the proximal handle extension 824 have a smaller outer effective diameter than at least an adjacent portion of the primary handle section 820 in the illustrated embodiment. Although the primary handle section 820, the distal extension 820a, and the proximal handle extension 824 could be separately formed and attached together in any appropriate manner (including where one or more of the primary handle section 820, the distal extension 820a, and the proximal handle extension 824 are formed from multiple sections), in the illustrated embodiment one body section 818 includes one portion of the primary handle section 820, the distal extension 820a, and the proximal handle extension 824, while the other body section 818 includes the opposing portion of the primary handle section 820, the distal extension 820a, and the proximal handle extension 824.

The interior of at least one of the body sections 818 includes a track section 818a. Each track section 818a may be of any appropriate length, including where a distal portion of the track section(s) 818a extend along the distal extension 820a (within an interior thereof), where the track section(s) 818a extend within an interior of the primary handle section 820 and along the entire length thereof, and where a proximal portion of the track section(s) 818a extend along the proximal handle extension 824 within an interior thereof. The distal extension 820a may incorporate a hole 822 (e.g., in one of the body sections 818), where such a hole 822 extends through the entire wall thickness of the distal extension 820a. Similarly, one or holes 826 may be spaced along the length dimension of the proximal handle extension 824 (e.g., in one of the body sections 818), where each such hole 822 extends through the entire wall thickness of the proximal handle extension 824. In the case where multiple holes 822 are spaced along the length of the proximal handle section 824 (including where one of the holes 822 may be located adjacent to (or closer to) a proximal end of the primary handle section 820), it may be that the distal extension 820a could be eliminated (in which case the track section(s) 818a may not be required for the primary handle section 820).

A shuttle 828 is disposed within the catheter handle 812, and is captured/guided in the track section 818a of at least one of the body sections 818. As such, relative motion between the shuttle 828 and the catheter handle 812 is at least substantially limited to relative axial motion (e.g., along the length dimension of the catheter handle 812). The shuttle 828 may be defined by two or more shuttle sections 830 that are appropriately attached to one another. A detent 832 is disposed within a receptacle 834 of the shuttle 828 that intersects with an outer perimeter of the shuttle 828. The detent 832 protrudes beyond the outer perimeter of the shuttle 828 and may be biased to this protruded position by one or more biasing members 834 of any appropriate type/configuration (e.g., a spring).

A proximal end of the catheter shaft 436 may be appropriately secured to the shuttle 828 (the catheter shaft 436 extending distally from the shuttle 828). One or more wires (not shown) may be appropriately secured to the shuttle 828, and may extend proximally from the shuttle 828 and out the proximal end 816 of the catheter handle 812 for interconnection with a plug that can attached to a generator cable or the like (e.g., generator cable 434). Such a plug may remain disposed proximally beyond the proximal handle extension 824 for all positions of the shuttle 828 (including when the shuttle 828 is located within the distal extension 820a). In any case, moving the shuttle 828 relative to the catheter handle 812 will change the working length of the catheter 436 (and similarly changes the spacing between the distal end 814 of the catheter handle 812 and the vasculature access to a patient if the catheter shaft 436 has been directed into the vasculature of the patient).

In the case where the detent 832 is positioned within one of the holes 822, 826 of the catheter handle 812, a user may depress the detent 832 in order to allow the shuttle 828 to be moved relative to the catheter handle 812. Advancing the shuttle 828 in a distal direction, relative to the catheter handle 812, will increase the working length of the catheter shaft 436 (and similarly increases the spacing between the distal end 814 of the catheter handle 812 and the vasculature access to a patient if the catheter shaft 436 has been directed into the vasculature of the patient). The shuttle 828 may be maintained in a fixed position relative to the catheter handle 812, after the above-noted relative movement, by aligning the detent 832 with one of the holes 822, 826 in the catheter handle 812. For instance, the detent 832 may initially have been positioned in a hole 826 of the proximal handle extension 824, and the shuttle 828 be advanced distally in the above-noted manner to align the detent 832 with a hole 822 in the primary handle section 820. In the illustrated embodiment, the shuttle 828 engages the distal end 814 of the catheter handle 812 to align the detent 832 of the shuttle 828 with the hole 826 in the distal extension 820a of the primary handle section 820.

Advancing the shuttle 828 in a proximal direction, relative to the catheter handle 812, will decrease the working length of the catheter shaft 436 (and similarly decreases the spacing between the distal end 814 of the catheter handle 812 and the vasculature access to a patient if the catheter shaft 436 has been directed into the vasculature of the patient). The shuttle 828 may be maintained in a fixed position relative to the catheter handle 812, after the above-noted relative movement, by aligning the detent 832 with one of the holes 822, 826 in the catheter handle 812. For instance, the detent 832 may initially have been positioned in a hole 822 in the distal extension 820a, and the shuttle 828 be advanced proximally in the above-noted manner, to align the detent 832 with a hole 826 of the proximal handle extension 824. In the illustrated embodiment, the shuttle 828 engages the proximal end 816 of the catheter handle 812 to align the detent 832 of the shuttle 828 with a hole 826 in the proximal handle extension 824.

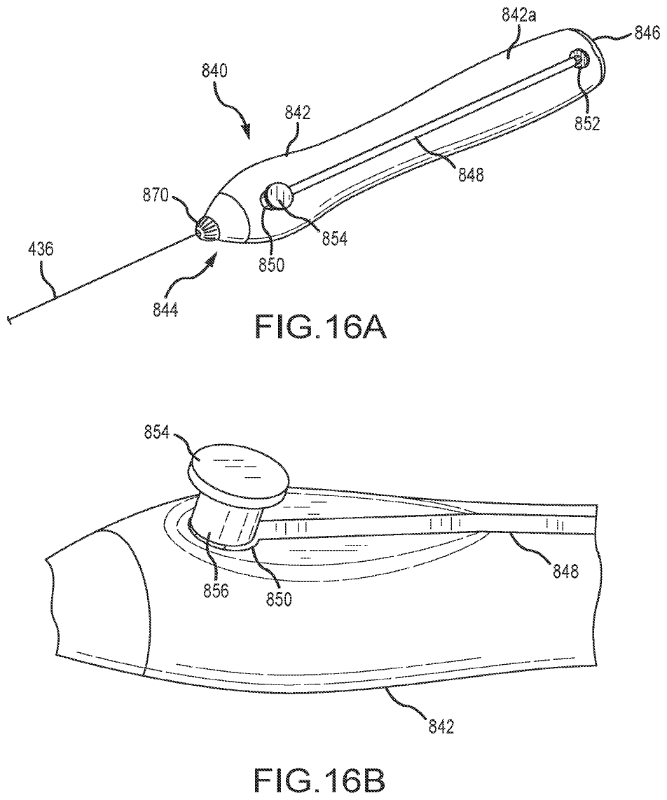

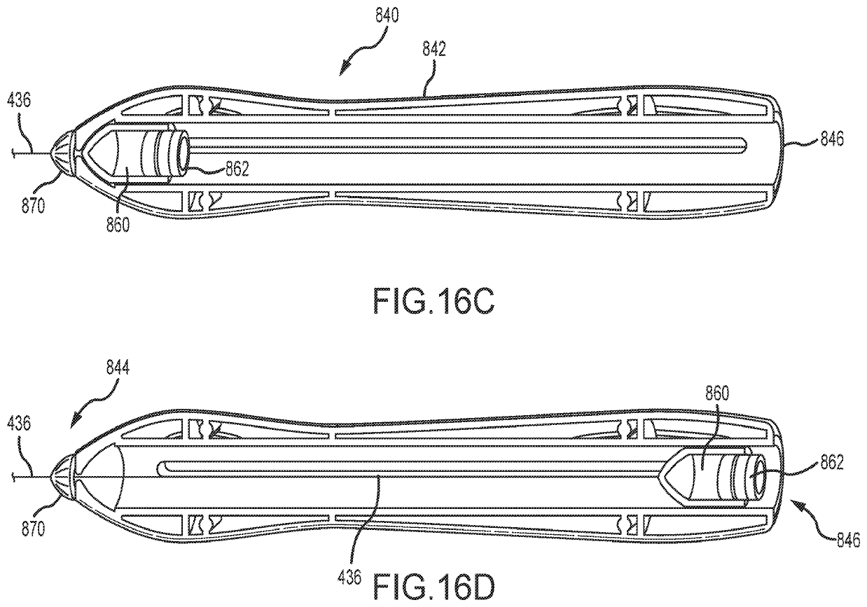

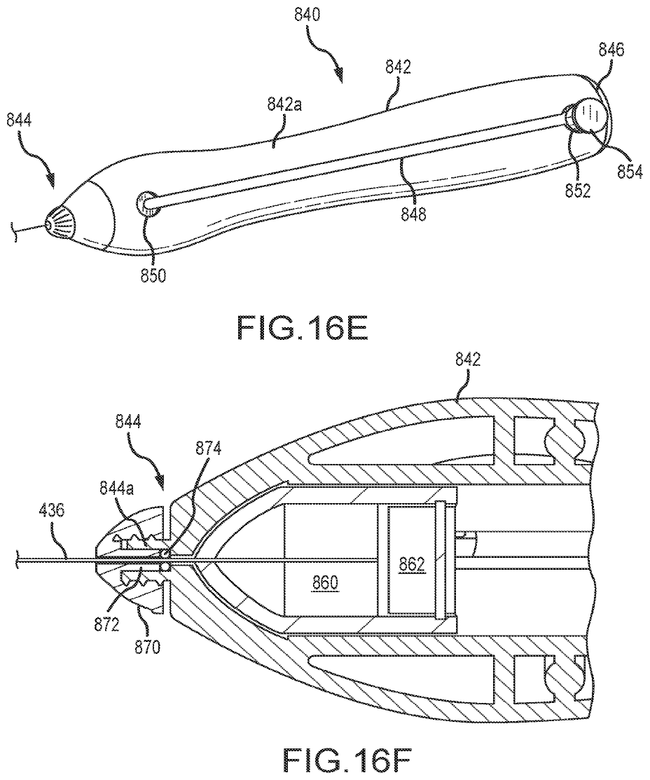

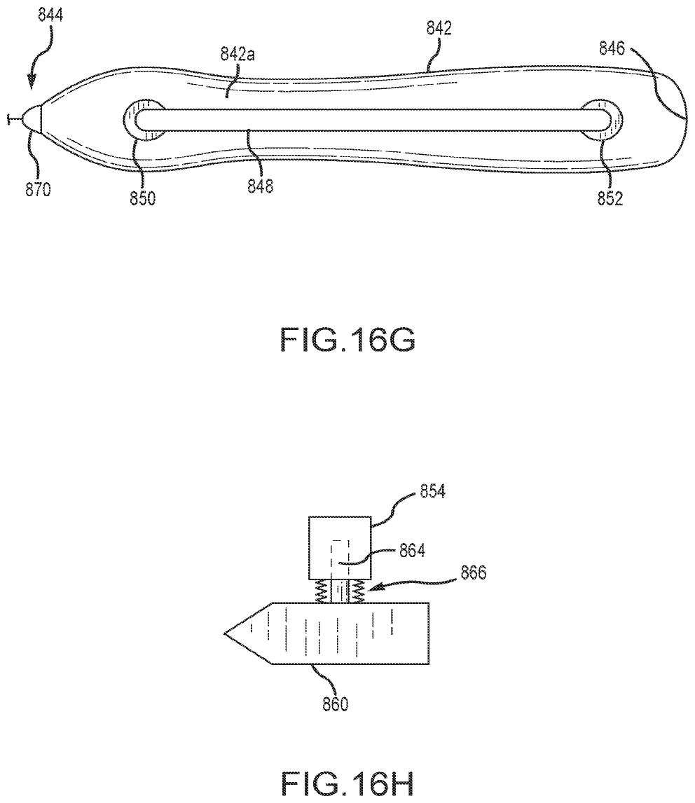

An embodiment of a catheter is illustrated in FIGS. 16A-16F is identified by reference numeral 840. The catheter 840 may be characterized as including a catheter handle 842 and a catheter shaft such as the above-noted catheter shaft 436. The catheter handle 842 includes a distal end 844 and a proximal end 846 that are spaced along a length dimension of the catheter handle 842. The catheter shaft 436 extends into the distal end 844 of the catheter handle 842, or stated another way the catheter shaft 436 extends distally from the distal end 844 of the catheter handle 842.

The catheter handle 842 includes a clamping actuator 870 that is disposed at the distal end 844 of the catheter handle 842 and that is rotatably interconnected with the catheter handle 842 (e.g., via a threaded engagement with a distal extension 844a at the distal end 844 of the catheter handle 842). The catheter shaft 436 extends through this clamping actuator 870 and through a clamping ring 874. The clamping ring 874 is disposed within an interior cavity of the distal extension 844a and is seated against a corresponding portion of the distal end 844 of the catheter handle 842 (and through which the catheter shaft 436 extends). The clamping actuator 870 includes a protrusion 872 that extends within the interior cavity of the distal extension 844a.

The clamping actuator 870 may be moved relative to the catheter handle 842 to change the clamping ring 874 of the catheter 840 between a clamped configuration (relative to the catheter shaft 436) and a released configuration (relative to the catheter shaft 436). The clamping actuator 870 is located at/near the distal end 844 of the catheter handle 842 in the illustrated embodiment, and may be rotated relative to the catheter handle 842 to change the clamping ring 874 between its clamped configuration and its released configuration.

Rotation of the clamping actuator 870 relative to the catheter handle 842 in one relative direction will compress the clamping ring 874 between the distal end 844 of the catheter handle 842 and the protrusion 872 of the clamping actuator 870 (the protrusion 872 of the clamping actuator 870 moving toward the proximal end 846 of the catheter handle 842), which in turn will dispose the clamping ring 874 in its clamped configuration relative to the catheter shaft 436. When the clamping actuator 870 is rotated in the noted manner to dispose the clamping ring 874 in its clamped configuration, the catheter handle 842 should be maintained in a fixed position relative to the catheter shaft 436 such that the catheter handle 842 may be used to move/manipulate the catheter shaft 436 relative to the patient's vasculature. Rotation of the clamping actuator 870 relative to the catheter handle 842 in the opposite relative direction to that described above will allow the clamping ring 874 to expand (the protrusion 872 of the clamping actuator 870 now move away from the proximal end 846 of the catheter handle 842), which in turn will dispose the clamping ring 874 in its released configuration. The released configuration allows the catheter handle 842 to be moved along and relative to the catheter shaft 436 to dispose the catheter handle 842 in a desired position relative to the catheter shaft 436 (e.g., to provide a desired working length for the catheter shaft 436). Typically it is the catheter handle 842 that is moved, with the catheter shaft 436 remaining stationary during this adjustment of the noted relative position. Once the catheter handle 842 is in the desired position along the length of the catheter shaft 436, the clamping actuator 870 may be rotated relative to the catheter handle 532 to dispose the clamping ring 874 back into its clamped configuration.

A slot 848 extends through a perimeter wall 842a of the catheter handle 842, may be characterized as being at least generally axially or linearly extending, may be of any appropriate length, and defines the amount of change available to the working length of the catheter shaft 436 in the case of the catheter 840. A first or distal aperture 850 (having a bottom wall—a “false bottom”) may be disposed at a distal end of the slot 848, and may be associated with the catheter 844 being used for a radial access to a patient's vasculature. A second or proximal aperture 852 (having a bottom wall—a “false bottom”) may be disposed at a proximal end of the slot 848, and may be associated with the catheter 844 being used for a femoral access to a patient's vasculature. One or more apertures (that intersect the slot 848—not shown) could be disposed between the distal aperture 850 and the proximal aperture 852.

A shuttle 860 is disposed within the catheter handle 842, and is movable relative to the catheter handle 842 along a path that is at least generally linearly or axially extending. As such, relative motion between the shuttle 860 and the catheter handle 842 is at least substantially limited to relative axial/liner motion (e.g., along the length dimension of the catheter handle 842). A proximal end of the catheter shaft 436 may be appropriately secured to the shuttle 860 (the catheter shaft 436 extending distally from the shuttle 860). The shuttle 860 may also include a connector 862 for providing an electrical connection between a generator (or another electrical component) and the catheter shaft 436. One or more wires (not shown) may be appropriately secured to the connector 862 and/or the catheter shaft 436, and may extend proximally from the shuttle 860 and out the proximal end 846 of the catheter handle 842 for interconnection with a generator cable (e.g., generator cable 434) or one or more other electrical components. In any case, moving the shuttle 860 relative to the catheter handle 842 will change the working length of the catheter 436 (and similarly changes the spacing between the distal end 844 of the catheter handle 842 and the vasculature access to a patient if the catheter shaft 436 has been directed into the vasculature of the patient).

The catheter handle 842 further includes a knob 854 that may be used in the adjustment of the position of the shuttle 860 along the length dimension of catheter handle 842. In this regard, a shaft 864 may extend from the shuttle 860, and the knob 854 may be slidably interconnected with this shaft 864. This shaft 864 and/or a corresponding portion of the knob 854 may extend through the slot 848 on the perimeter wall 842a of the catheter handle 842. One or biasing members 866 may bias the knob 854 into engagement with the catheter handle 842. The knob 854 may be pulled away from the catheter handle 842 to move the knob 854 along the slot 848 and that will provide a corresponding movement of the shuttle 860 relative to the catheter handle 842.

The knob 854 may also be used to lock the position of the shuttle 860 relative to the catheter handle 842. In this regard, a body 856 of the knob 854 may be disposed in either the distal aperture 850 (and seated on its bottom wall) or in the proximal aperture 852 (and seated on its bottom wall). A portion of the body 856 of the knob 854 that is disposable in the apertures 850, 852 may be correspondingly-shaped with the apertures 850, 852. As such, the knob 854 may also be referred to as a lock 854. This, in combination with using the clamping actuator 870 to dispose the clamping ring 874 in its clamped configuration, may be used to maintain the catheter shaft 436 in a fixed position relative to the catheter handle 842.

With the body 856 of the knob 854 being disposed within the proximal aperture 852, the clamping actuator 870 may be rotated to dispose the clamping ring 874 in its released configuration relative to the catheter shaft 436. In addition, the knob 854 may be pulled away from the perimeter wall 842a of the catheter handle 842 (to dispose its body 856 out of the proximal aperture 852). The catheter handle 842 may then be moved relative to the catheter shaft 436 such that the knob 854 is moved distally along the slot 848 (typically by moving the catheter handle 842 in a proximal direction, with the catheter shaft 436, the shuttle 860, and knob 854 remaining in a stationary position). This relative movement will increase the working length of the catheter shaft 436 and similarly increases the spacing between the distal end 844 of the catheter handle 842 and the vasculature access to a patient if the catheter shaft 436 has been directed into the vasculature of the patient. The body 856 of the knob 854 may be disposed in the distal aperture 850 (and may be seated on its corresponding bottom wall). Moreover, the clamping actuator 872 may be rotated to dispose the clamping ring 874 its clamped configuration relative to the catheter shaft 436. At this time, the catheter handle 842 may be used to move/manipulate the catheter shaft 436 relative to the patient's vasculature, as the catheter shaft 436 should be maintained in a fixed position relative to the catheter handle 842.

With the body 856 of the knob 854 being disposed within the distal aperture 850, the clamping actuator 872 may be rotated to dispose the clamping ring 874 in its released configuration relative to the catheter shaft 436. In addition, the knob 854 may be pulled away from the perimeter wall 842a of the catheter handle 842 (to dispose its body 856 out of the distal aperture 850). The catheter handle 842 may then be moved relative to the catheter shaft 436 such that the knob 854 is moved proximally along the slot 848 (typically by moving the catheter handle 842 in a distal direction, with the catheter shaft 436, the shuttle 860, and knob 854 remaining in a stationary position). This relative movement will decrease the working length of the catheter shaft 436 and similarly decreases the spacing between the distal end 844 of the catheter handle 842 and the vasculature access to a patient if the catheter shaft 436 has been directed into the vasculature of the patient. The body 856 of the knob 854 may be disposed in the proximal aperture 852 (and may be seated on its corresponding bottom wall). Moreover, the clamping actuator 872 may be rotated to dispose the clamping ring 874 its clamped configuration relative to the catheter shaft 436. At this time, the catheter handle 842 may be used to move/manipulate the catheter shaft 436 relative to the patient's vasculature, as the catheter shaft 436 should be maintained in a fixed position relative to the catheter handle 842.

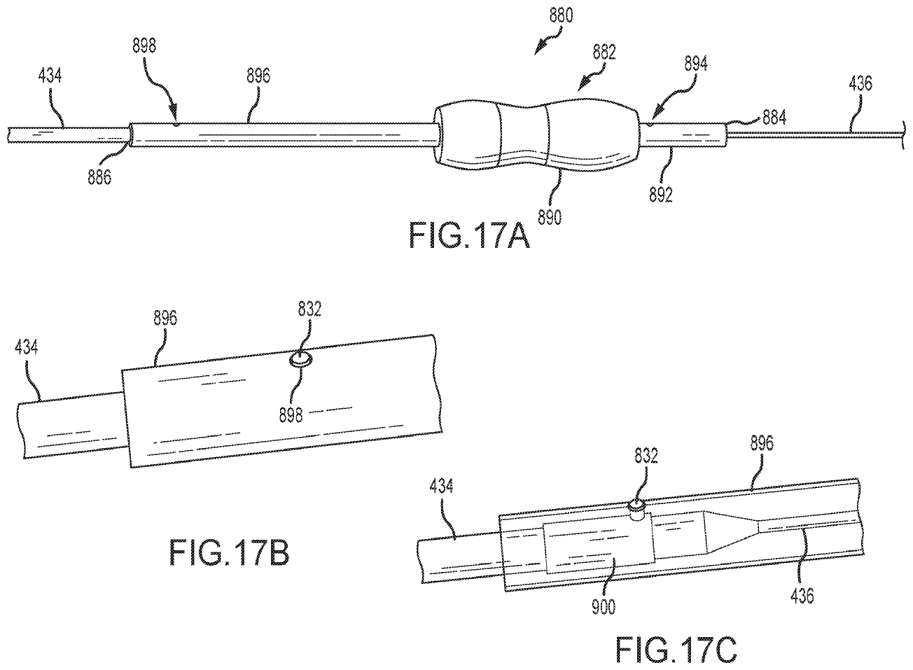

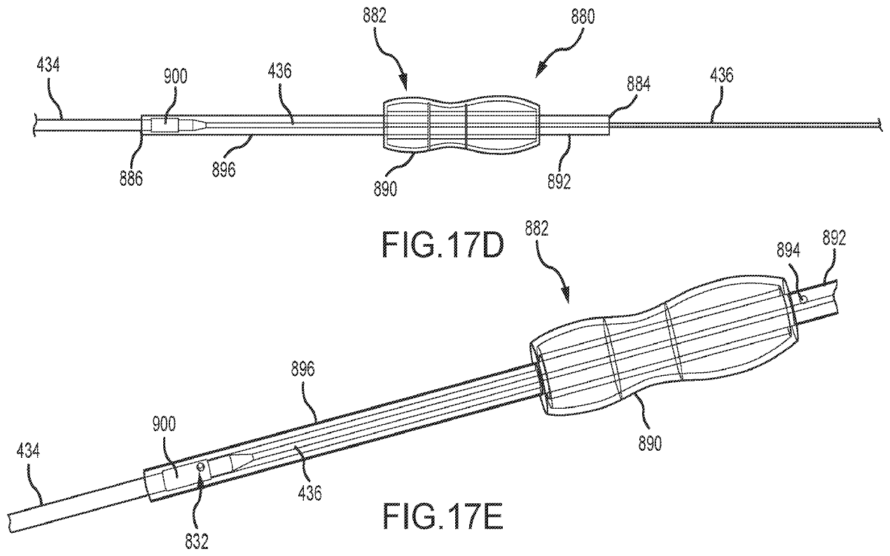

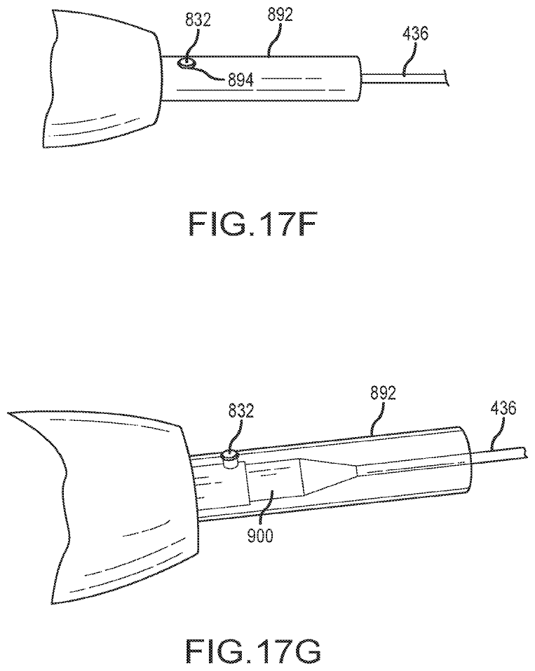

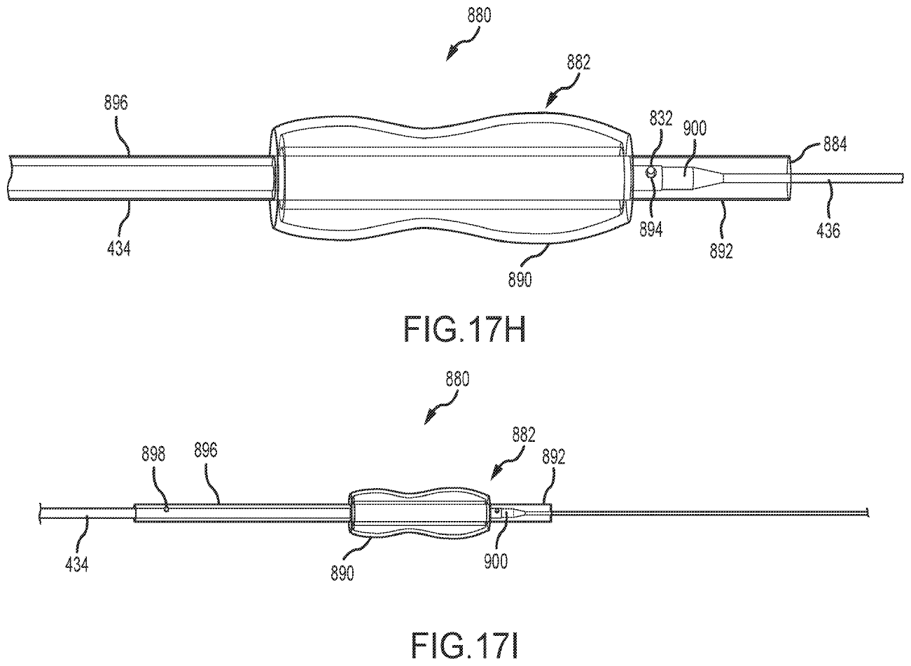

An embodiment of a catheter is illustrated in FIGS. 17A-17I and is identified by reference numeral 880. The catheter 880 may be characterized as including a catheter handle 882 and a catheter shaft such as the above-noted catheter shaft 436. A catheter shaft clamp (e.g., in accord with any of the various embodiments described herein) could also be utilized by the catheter 880.

The catheter handle 882 includes a distal end 884 and a proximal end 886 that are spaced along a length dimension of the catheter handle 882. There are three primary portions of the catheter handle 882—a primary handle section 890 (engaged by a user of the catheter 880, for instance to manipulate the catheter shaft 436 relative to a patient; e.g., in accordance with the catheter handle 492/body 494 discussed above in relation to FIG. 11A1 in relation to the shape/contour of the outer perimeter of the primary handle section 890), a distal shaft or extension 892 (that includes the noted distal end 880), and a proximal shaft or extension 896 (that includes the noted proximal end 886). The distal shaft 892 is maintained in a fixed position relative to and extends distally from the primary handle section 890. Similarly, the proximal shaft 896 is maintained in a fixed position relative to extends proximally from the primary handle section 890. Any appropriate way of making/assembling a catheter handle 882 having the above-noted features/configuration may be utilized. One or holes 894 may be spaced along the length dimension of the distal shaft 892, where each such hole 894 extends through the entire wall thickness of the distal shaft 892. Similarly, one or holes 898 may be spaced along the length dimension of the proximal shaft 896, where each such hole 898 extends through the entire wall thickness of the proximal shaft 896. Both the distal shaft/extension 892 and the proximal shaft/extension 896 have a smaller outer effective diameter than at least an adjacent portion of the primary handle section 890 in the illustrated embodiment.

A shuttle 900 is disposed within the catheter handle 882 and is movable relative to the catheter handle 882 along its length dimension, including along/relative to each of the distal shaft 892 and the proximal shaft 896 (e.g., the shuttle 900 may move at least generally along an axial path relative to the catheter handle 882). The shuttle 900 provides a movable interconnection between the catheter shaft 436 (which is appropriately attached/secured to the shuttle 900 and extends distally from the shuttle 900 and beyond the distal end 884 of the catheter handle 882) and the above-noted generator cable 434 (which extends proximally from the shuttle 900 and beyond the proximal end 884 of the catheter handle 882). For instance, the shuttle 900 could include a printed circuit board or the like. As such, moving the shuttle 900 relative to the catheter handle 882 will change the working length of the catheter 436 (and similarly changes the spacing between the distal end 814 of the catheter handle 882 and the vasculature access to a patient if the catheter shaft 436 has been directed into the vasculature of the patient). Typically it is the catheter handle 882 that is moved, with the catheter shaft 436, shuttle 900, and generator cable 434 remaining stationary during this adjustment of the noted relative position.

The shuttle 900 includes above-described detent 832, which is disposed within and is biased to protrude beyond an outer perimeter of the shuttle 900 (when not compressed). In the case where the detent 832 is positioned within one of the holes 894, 898 of the catheter handle 882, a user may depress the detent 832 in order to allow the catheter handle 882 to be moved relative to the shuttle 900. Advancing the shuttle 900 in a distal direction, relative to the catheter handle 882 (typically by moving the catheter handle 882 in a proximal direction), will increase the working length of the catheter shaft 436 (and similarly increases the spacing between the distal end 884 of the catheter handle 882 and the vasculature access to a patient if the catheter shaft 436 has been directed into the vasculature of the patient). The shuttle 900 may be maintained in a fixed position relative to the catheter handle 882, after the above-noted relative movement, by aligning the detent 832 with one of the holes 894, 898 in the catheter handle 812. For instance, the detent 832 may initially have been positioned in a hole 898 of the proximal shaft 896, and the shuttle 900 be advanced distally in the above-noted manner to align the detent 832 with a hole 894 in the distal shaft 892.

Advancing the shuttle 900 in a proximal direction, relative to the catheter handle 882(typically by moving the catheter handle 882 in a distal direction), will decrease the working length of the catheter shaft 436 (and similarly decreases the spacing between the distal end 884 of the catheter handle 882 and the vasculature access to a patient if the catheter shaft 436 has been directed into the vasculature of the patient). The shuttle 900 may be maintained in a fixed position relative to the catheter handle 882, after the above-noted relative movement, by aligning the detent 832 with one of the holes 894, 898 in the catheter handle 812. For instance, the detent 832 may initially have been positioned in a hole 894 in the distal shaft 892, and the shuttle 900 be advanced proximally in the above-noted manner, to align the detent 832 with a hole 898 in the proximal shaft 896.

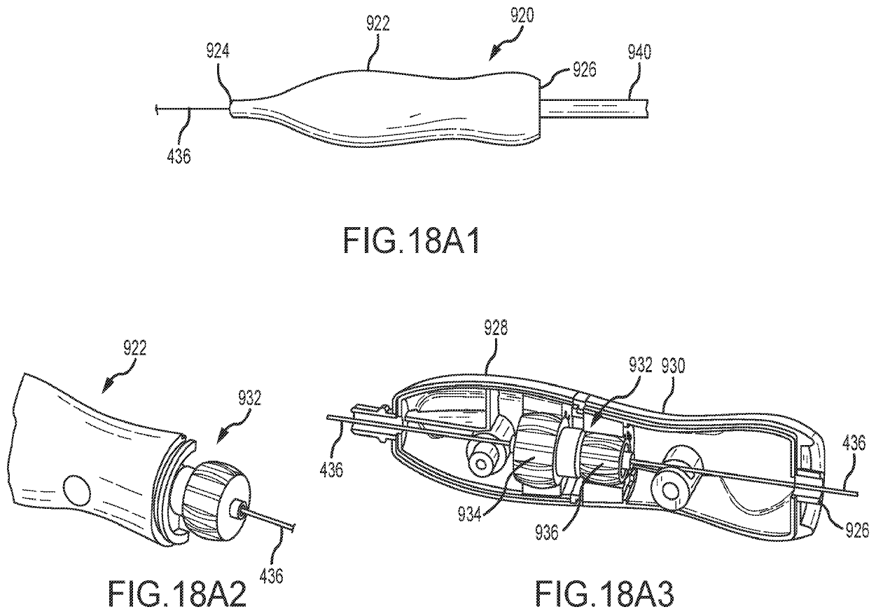

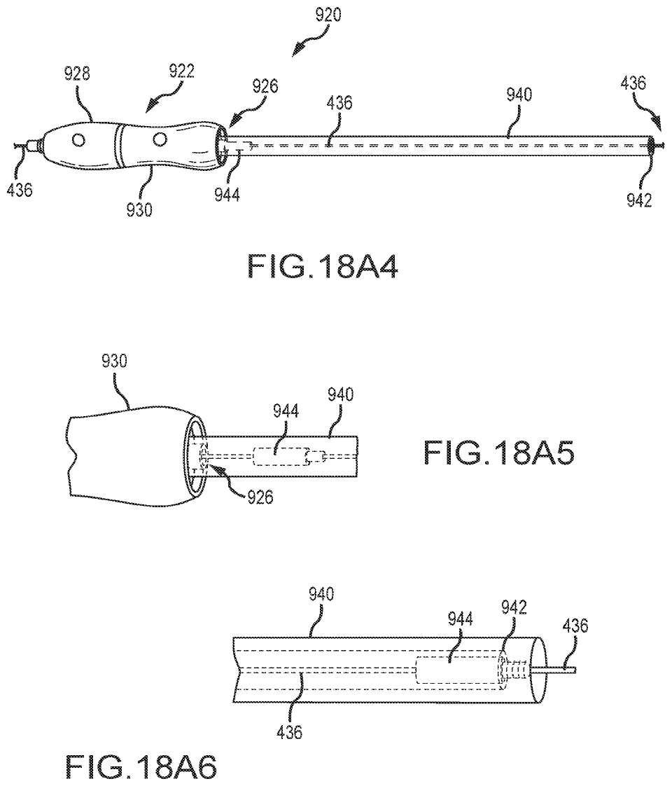

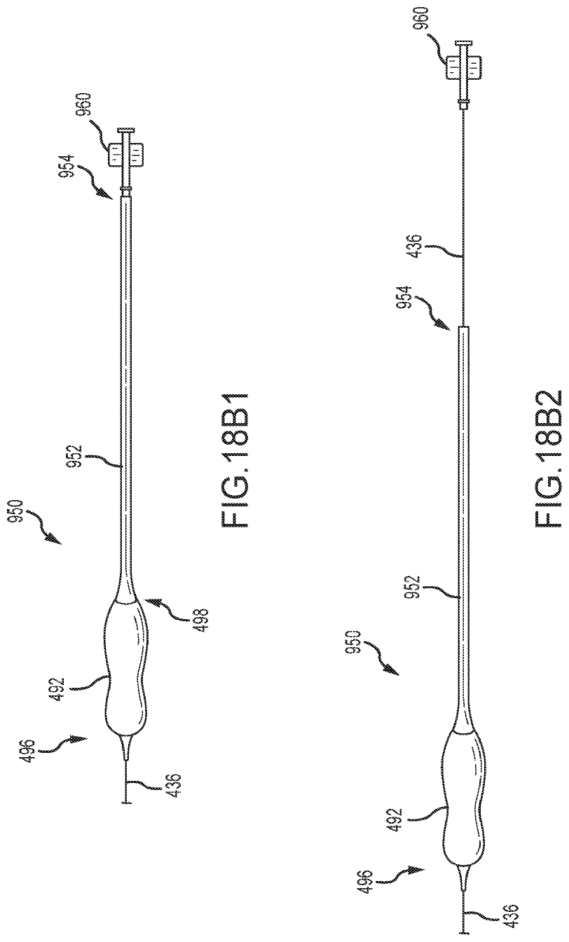

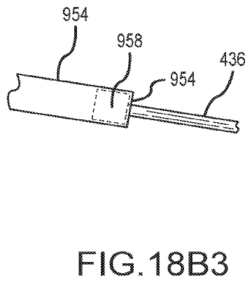

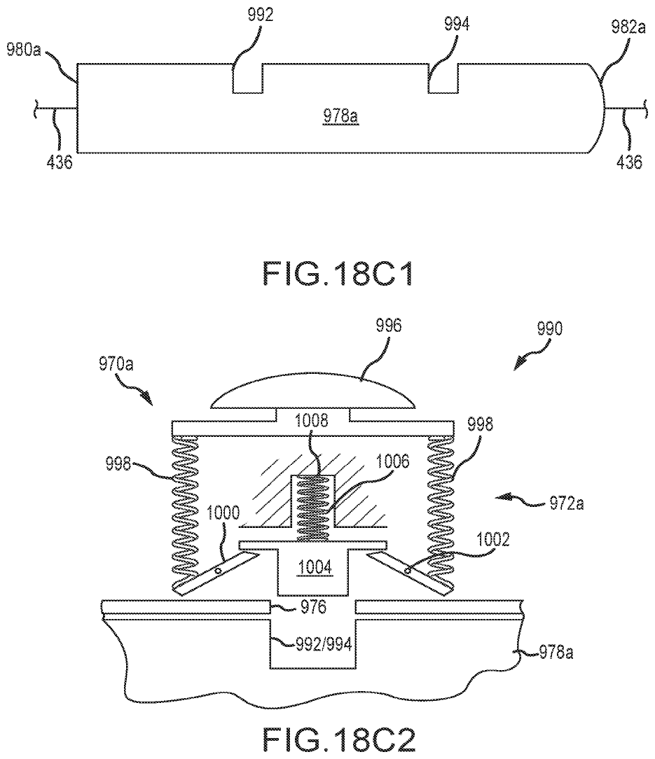

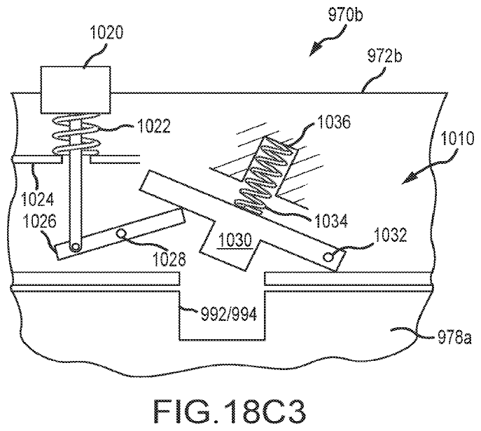

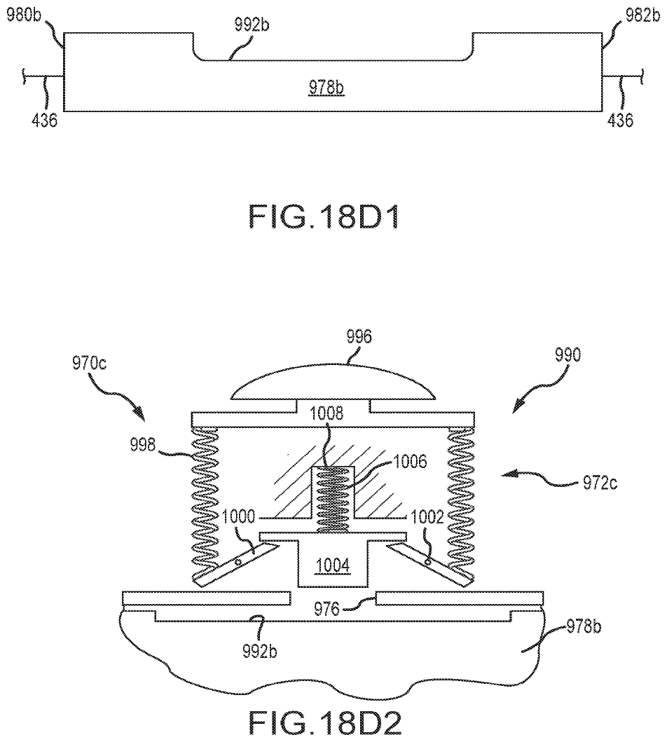

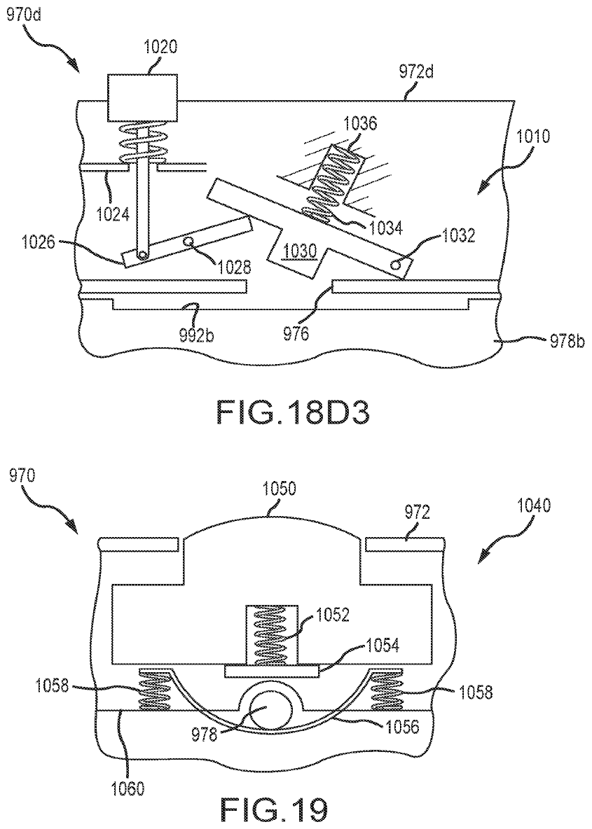

An embodiment of a catheter is illustrated in FIGS. 18A1-18A6 and is identified by reference numeral 920. The catheter 920 may be characterized as including a catheter handle 922, a catheter shaft such as the above-noted catheter shaft 436, and a proximal sleeve or shaft 940. The catheter handle 922 includes a distal end 924 and a proximal end 926 that are spaced along a length dimension of the catheter handle 922. There are two primary portions of the catheter handle 922—a distal handle section 928 and a proximal handle section 930 that are rotatable relative to one another. A catheter shaft clamp 932 (e.g., a Touhy mechanism) is disposed within the catheter handle 922 and includes a distal section 934 and a proximal section 936. The distal section 934 of the catheter shaft clamp 932 is associated with the distal handle section 928, while the proximal section 936 of the catheter shaft clamp 932 is associated with the proximal handle section 930. Rotation of the distal handle section 928 relative to the proximal handle section 930 in one direction rotates the distal section 934 of the catheter shaft clamp 932 relative to the proximal section 936 of the catheter shaft clamp 932 to dispose the catheter shaft clamp 932 in one of a clamped configuration or a released configuration (relative to the catheter shaft 436), for instance to compress a clamping ring (e.g., clamping ring 794; clamping ring 874). Rotation of the distal handle section 928 relative to the proximal handle section 930 in an opposite direction rotates the distal section 934 of the catheter shaft clamp 932 relative to the proximal section 936 of the catheter shaft clamp 932 to dispose the catheter shaft clamp 932 in the other of the clamped configuration or a released configuration (relative to the catheter shaft 436), for instance to allow a clamping ring to expand.

The proximal sleeve 940 is appropriately secured to and extends proximally from the catheter handle 922. The catheter shaft 436 extends beyond the proximal end 926 of the catheter handle 922, into the proximal sleeve 940, and through a proximal end 942 of the proximal sleeve 940. A stop 944 (e.g., a third stop, or a “shuttle”) is disposed within the proximal sleeve 940 and is fixed relative to a portion of the catheter shaft 436 that at all times remains disposed within the proximal sleeve 940. When the catheter shaft clamp 932 is disposed in its released configuration relative to the catheter shaft 436, the catheter shaft 436 is movable relative to both the catheter handle 922 and the proximal sleeve 940 (again, typically by moving the catheter handle 922 and proximal sleeve 940 relative to a stationary catheter shaft 436). The range of motion of the catheter shaft 436 relative to both the catheter handle 922 and the proximal sleeve 940 (and thereby defining the adjustable range for the working length of the catheter shaft 436) is defined by an engagement between the stop 944 (within the proximal sleeve 940 and on the catheter shaft 436) against the proximal end 926 of the catheter handle 922 (e.g., the proximal end 926 of the catheter handle 922 may be characterized as a “first stop”), and is defined by an engagement between the stop 944 (within the proximal sleeve 940 and on the catheter shaft 436) against the proximal end 942 of the proximal sleeve 940 (e.g., the proximal end 942 of the proximal sleeve 940 may be characterized as a “second stop”).

When it is desired to reposition the catheter handle 922 along the catheter shaft 436, the catheter shaft clamp 932 may be disposed in its released configuration and the catheter handle 922 may be moved relative to and along the catheter shaft 436 to dispose the catheter handle 922 in a desired position (e.g., to provide a desired working length for the catheter shaft 436; e.g., by moving the catheter handle 922 and proximal sleeve 940 relative to a stationary catheter shaft 436 and a stationary stop 944). Thereafter, the catheter shaft clamp 932 may be disposed in its clamped configuration (relative to the catheter shaft 436) such that the catheter handle 922 may be used to move/manipulate the catheter shaft relative to the patient's vasculature. Moving the catheter handle 922 relative to and along the catheter shaft 435 until the stop 944 (mounted on the catheter shaft 436 and remaining within the proximal sleeve 940) engages the proximal end 926 of the catheter handle 922, and thereafter disposing the catheter shaft clamp 932 in its clamped configuration provides the maximum working length for the catheter shaft 436 in the case of the catheter 920 (e.g., for radial access to a patient's vasculature). Moving the catheter handle 922 relative to and along the catheter shaft 436 until the stop 944 (mounted on the catheter shaft 436 and remaining within the proximal sleeve 940) engages the proximal end 942 of the proximal sleeve 940, and thereafter disposing the catheter shaft clamp 932 in its clamped configuration provides the minimum working length for the catheter shaft 436 in the case of the catheter 920 (e.g., for femoral access to a patient's vasculature). It should be appreciated that any working length for the catheter shaft 436 between these two extremes is available via the catheter 920 (the working length for the catheter shaft 436 being fixed by disposing the catheter shaft clamp 932 in its clamped configuration).

Another embodiment of a catheter is illustrated in FIGS. 18B1-18B3 and is identified by reference numeral 950. There are three primary components/subassemblies of the catheter 950—the above-described catheter handle 492 (FIGS. 11A1-11A4), a catheter shaft such as the above-noted catheter shaft 436, and a proximal sleeve or shaft 952. As discussed above, the catheter handle 492 includes a clamping actuator 506 and catheter shaft clamp 508, with the clamping actuator 506 being configured to dispose the catheter shaft clamp 508 in each of a clamped configuration (relative to the catheter shaft 436) and a released configuration (relative to the catheter shaft 436). However, any appropriate clamping actuator and catheter shaft clamp may be utilized by the catheter 950.

The proximal sleeve 952 is appropriately secured to and extends proximally from the proximal end 498 of the catheter handle 492. The catheter shaft 436 extends beyond the proximal end 498 of the catheter handle 492, into the proximal sleeve 952, and through a proximal end 954 of the proximal sleeve 952. A stop 958 remains disposed within the proximal sleeve 952 and is fixed relative to a portion of the catheter shaft 436 that remains disposed within the proximal sleeve 952. Another stop 960 is disposed beyond the proximal end 954 of the proximal sleeve 952 and is fixed relative to a portion of the catheter shaft 436 that remains disposed beyond the proximal end 954 of the proximal sleeve 952. The stops 958, 960, and the portion of the catheter shaft 436 extending therebetween, may be referred to as a “shuttle.”

When the catheter shaft clamp 508 is disposed in its released configuration relative to the catheter shaft 436, the catheter shaft 436 is movable relative to both the catheter handle 492 and the proximal sleeve 952. The range of motion of the catheter shaft 436 relative to both the catheter handle 492 and the proximal sleeve 952 (and thereby defining the adjustable range for the working length of the catheter shaft 436) is defined by an engagement between the stop 958 (within the proximal sleeve 940 and on the catheter shaft 436) against the interior side of proximal end 954 of the proximal sleeve 952 (e.g., the interior side of the proximal end 954 of the proximal sleeve 952 may be characterized as a “stop”), and is further defined by an engagement between the stop 960 (proximally beyond the proximal sleeve 952 and on the catheter shaft 436) against the exterior side of the proximal end 954 of the proximal sleeve 952 (e.g., the exterior side of the proximal end 954 of the proximal sleeve 952 may be characterized as a “stop”).

When it is desired to reposition the catheter handle 492 along the catheter shaft 436, the catheter shaft clamp 508 may be disposed in its released configuration and the catheter handle 492 may be moved relative to and along the catheter shaft 436 to dispose the catheter handle 492 in a desired position (e.g., to provide a desired working length for the catheter shaft 436; e.g., by moving the catheter handle 492 and proximal sleeve 952 relative to a stationary catheter shaft 436). Thereafter, the catheter shaft clamp 508 may be disposed in its clamped configuration (relative to the catheter shaft 436) and such that the catheter handle 492 may be used to move/manipulate the catheter shaft 436 relative to the patient's vasculature. Moving the catheter handle 492 relative to and along the catheter shaft 436 until the stop 960 (mounted on the catheter shaft 436 and disposed proximally beyond the proximal sleeve 952) engages the exterior side of the proximal end 954 of the proximal sleeve 952, and thereafter disposing the catheter shaft clamp 508 in its clamped configuration provides the maximum working length for the catheter shaft 436 in the case of the catheter 950 (e.g., for radial access to a patient's vasculature). Moving the catheter handle 492 relative to and along the catheter shaft 436 until the stop 958 (mounted on the catheter shaft 436 and remaining within the proximal sleeve 952) engages the interior side of the proximal end 954 of the proximal sleeve 952, and thereafter disposing the catheter shaft clamp 508 in its clamped configuration provides the minimum working length for the catheter shaft 436 in the case of the catheter 950 (e.g., for femoral access to a patient's vasculature). It should be appreciated that any working length for the catheter shaft 436 between these two extremes is available via the catheter 950 (the working length for the catheter shaft 436 being fixed by disposing the catheter shaft clamp 508 in its clamped configuration).

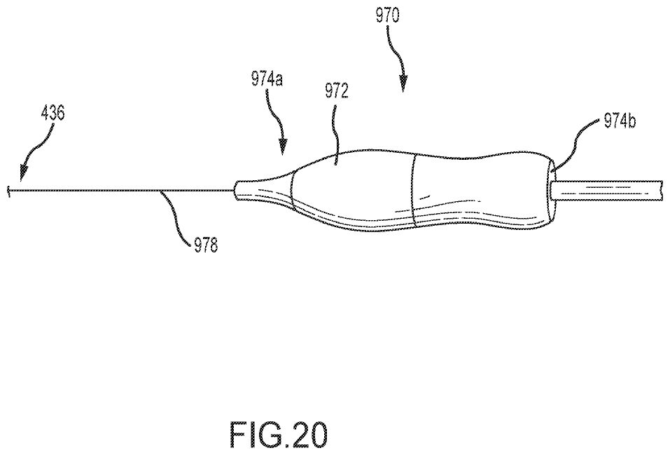

Another embodiment of a catheter is schematically illustrated in FIGS. 18C1-18C2 and is identified by reference numeral 970a (see also FIG. 20, discussed below). The catheter 970a includes a catheter handle 972a (schematically shown). A shaft 978a is movably disposed within the catheter handle 972a of the catheter 970a, includes a distal end 980a and a proximal end 982a, and further includes a first recess or notch 992 (e.g., distally disposed) and a second recess or notch 994 (e.g., proximally disposed). Any number of recesses may be utilized. The recesses 992, 994 are spaced from one another along a length dimension of the catheter handle 972a (e.g., corresponding with the spacing between the distal end 980a and proximal end 982a of the shaft 978a). A catheter shaft 436 (see also FIG. 20) extends distally from the distal end 980a of the shaft 978a, and may also extend proximally a short distance from the proximal end 982a of the shaft 978a.

The catheter handle 972a includes what may be characterized as a first clamping arrangement 990. The catheter handle 972a includes a release button 996 that is accessible on an exterior of the catheter handle 972a. One or more biasing members 998 of any appropriate type/configuration (e.g., a spring) extend between the release button 996 and one or more levers 1000. Each lever 1000 is interconnected with the catheter handle 972a by a pivot 1002. The first clamping arrangement 990 further includes what may be characterized as a detent or a catch 1004 that is biased to an engaged position with the shaft 978a by a biasing member 1006 (one end of the biasing member 1006 abutting an anchor 1008 that is maintained in a fixed position relative to the catheter handle 972a). When the detent 1004 is aligned with one of the recesses 992, 994 of the shaft 978a, the biasing member 1006 will dispose the detent 1004 in the corresponding recess 992, 994, absent actuation of the release button 996. This may be characterized as a clamped configuration for the shaft 978a, such that the catheter handle 972a may be used to manipulate the catheter shaft 436 (e.g., the catheter shaft 436 should be maintained in a fixed position relative to the catheter handle 972a). When the release button 996 is actuated (e.g., moved at least generally in the direction of the shaft 978a), the biasing member(s) 998 will exert a force on the corresponding lever(s) 1000 and in a direction that pivots the lever(s) 1000 relative to the catheter handle 972a that disposes the detent 1004 out of the corresponding recess 992, 994. This may be characterized as disposing the shaft 978a in a released configuration (or a released configuration for the catheter shaft 436) and that allows the catheter handle 972a to be moved relative to the shaft 978a (and thereby the catheter shaft 436). This in turn changes the working length of the catheter shaft 436.

Another embodiment of a catheter is schematically illustrated in FIGS. 18C1 and 18C3 and is identified by reference numeral 970b (see also FIG. 20, discussed below). The catheter 970b includes a catheter handle 972b (schematically shown), the shaft 978a, and a catheter shaft 436. The above-noted shaft 978a is movably disposed within the catheter handle 972b of the catheter 970b. The catheter handle 972b includes what may be characterized as a second clamping arrangement 1010.

The catheter handle 972b, with its second clamping arrangement 1010, includes a release button 1020 that is accessible on an exterior of the catheter handle 972b. A biasing member 1022 of any appropriate type/configuration (e.g., a spring) extends between the release button 1020 and an anchor 1024 that is disposed in a fixed position relative to the catheter handle 972b. The release button 1020 extends to a lever 1026 that is pivotally connected with the catheter handle 972b by a pivot 1028.

The second clamping arrangement 1010 further includes what may be characterized as a detent or a catch 1030 that is pivotally connected with the catheter handle 972b by a pivot 1032. The detent 1030 is biased to an engaged position with the shaft 978a by a biasing member 1034 (one end of the biasing member 1034 abutting an anchor 1036 that is maintained in a fixed position relative to the catheter handle 972b). When the detent 1004 is aligned with one of the recesses 992, 994 of the shaft 978a, the biasing member 1034 will dispose the detent 1030 in the corresponding recess 992, 994, absent actuation of the release button 1020. This may be characterized as a clamped configuration for the shaft 978a, such that the catheter handle 972b may be used to manipulate the catheter shaft 436 (e.g., the catheter shaft 436 should be maintained in a fixed position relative to the catheter handle 972b). When the release button 1020 is actuated (e.g., moved at least generally in the direction of the shaft 978a), the release button 1020 exerts a force on the lever 1026 to pivot the same (relative to the catheter handle 972b) in a direction that disposes the detent 1030 out of the corresponding recess 992, 994. This may be characterized as disposing the shaft 978a in a released configuration and that allows the catheter handle 972b to be moved relative to the shaft 978a. This in turn changes the working length of the catheter shaft 436.

Another embodiment of a catheter is schematically illustrated in FIGS. 18D1-18D2 and is identified by reference numeral 970c (see also FIG. 20, discussed below). The catheter 970c includes a catheter handle 972c (schematically shown). A shaft 978b is movably disposed within the catheter handle 972c of the catheter 970c. Instead of using two or more recesses 992, 994, the shaft 978b includes an elongate recess 992b on its perimeter. The catheter handle 972c uses the above-described clamping arrangement 990, where the detent 1004 may be clamped against the shaft 978b anywhere along the length dimension of the elongate recess 992b. This may be characterized as a clamped configuration for the shaft 978b, such that the catheter handle 972c may be used to manipulate the catheter shaft 436 (e.g., the catheter shaft 436 should be maintained in a fixed position relative to the catheter handle 972c). A released configuration for the shaft 978b would be in accord with the discussion presented above with regard to FIGS. 18C1-18C2.

Another embodiment of a catheter is schematically illustrated in FIGS. 18D1 and 18D3 and is identified by reference numeral 970d (see also FIG. 20, discussed below). The catheter 970d includes a catheter handle 972d (schematically shown). The above-noted shaft 978b is movably disposed within the catheter handle 972d of the catheter 970d. The catheter handle 972d uses the above-described clamping arrangement 1010, where the detent 1030 may be clamped against the shaft 978b anywhere along the length dimension of the elongate recess 992b. This may be characterized as a clamped configuration for the shaft 978b, such that the catheter handle 972d may be used to manipulate the catheter shaft 436 (e.g., the catheter shaft 436 should be maintained in a fixed position relative to the catheter handle 972d). A released configuration for the shaft 978b would be in accord with the discussion presented above with regard to FIGS. 18C1 and 18C3.

Another embodiment of a catheter is schematically illustrated in FIG. 19 and is identified by reference numeral 970 (see also FIG. 20, discussed below). The catheter 970 includes a catheter handle 972 (schematically shown). A shaft 978 (e.g., a proximal portion of a catheter shaft) is movably disposed within the catheter handle 972 of the catheter 970. Unlike the above-noted shafts 978a, 978b, the shaft 978 does not include any recesses or an elongate recess on its outer perimeter. Instead, the catheter handle 972 uses a clamping arrangement 1040 that does not require multiple recesses or an elongate recess for the shaft 978.

With regard to the clamping arrangement 1040, the catheter handle 972 includes a release actuator or button 1050 that is accessible on an exterior of the catheter handle 972. A biasing member 1052 of any appropriate type/configuration (e.g., a spring) extends between the release button 1050 and an anchor 1054 that is maintained in a fixed position relative to the catheter handle 972. The clamping arrangement 1040 further includes a catheter shaft clamp 1056 that is biased to an engaged position with the shaft 978 by one or more biasing members 1058 of any appropriate type or configuration (e.g., a spring). In the illustrated embodiment, the release button 1050 and the portion of the catheter shaft clamp 1056 that engages the shaft 978 (e.g., a curved/concave surface of the catheter shaft clamp 1056) are disposed on opposite sides of the shaft 978. The portion of the catheter shaft clamp 1056 that engages the shaft 978 may be at least generally correspondingly-shaped with an engaged portion of the shaft 978. In any case, one end of each biasing member 1058 abuts an anchor 1060 that is maintained in a fixed position relative to the catheter handle 972). Absent actuation of the release button 1050, the biasing member(s) 1058 will dispose the catheter shaft clamp 1056 against the shaft 978 with sufficient force so as to maintain the shaft 978 in a fixed position relative to the catheter handle 972. This may be characterized as a clamped configuration for the shaft 978 (and thereby a corresponding catheter shaft), such that the catheter handle 972 may be used to manipulate the catheter shaft (e.g., the catheter shaft should be maintained in a fixed position relative to the catheter handle 972). When the release button 1050 is actuated (e.g., moved at least generally in the direction of the shaft 978), the release button 1050 will compress the biasing member(s) 1058 to at least reduce the engagement between the catheter shaft clamp 1056 and the shaft 978 (or to completely disengage the catheter shaft clamp 1056 from the shaft 978). This may be characterized as disposing the shaft 978 in a released configuration (or a released configuration for the catheter shaft that extends distally of the shaft 978) and that allows the catheter handle 972 to be moved relative to the shaft 978. This in turn changes the working length of the catheter shaft.

FIG. 20 illustrates a catheter 970 that may be adapted in accord with the embodiments of FIGS. 18C1-19. The catheter 970 includes a catheter handle 972 having a distal end 974a and a proximal end 974. The catheter 970 further includes a shaft 978 that is shown as extending distally from the distal end 974a of the catheter handle 972, as well as a catheter shaft 436 that extends distally from this proximal shaft 978 (the proximal shaft 978 could simply be viewed as a proximal portion of the catheter shaft 436). A generator cable may extend proximally from the proximal end 974 of the catheter handle 972.

Each of the various embodiments shown in FIGS. 5A-19 may be characterized as a catheter shaft actuator that may be used in combination with a catheter shaft, where this catheter shaft provides any appropriate function or combination of functions, and where this catheter shaft actuator accommodates varying a working length of the catheter shaft—the working length being distance between a distal end of the catheter shaft and a distal end of the catheter shaft actuator. Each such catheter shaft actuator may be used to manipulate the catheter shaft in any appropriate relative to the patient's vasculature and including to advance the catheter along the patient's vasculature.

The ability to move a catheter shaft actuator along/relative to the catheter shaft may be used for any appropriate purpose. For instance, the catheter shaft actuator may be disposed in a first position relative to the distal end of the catheter, a catheter shaft clamp could then be disposed in a clamped configuration, and the catheter shaft actuator could thereafter be used to advance the catheter shaft relative to the patient's vasculature a desired distance/amount. Thereafter, the catheter shaft clamp could be disposed in it released configuration, the catheter shaft actuator could be moved proximally along the catheter shaft and thereby away from the access to the patient's vasculature to a desired position, and thereafter the catheter shaft clamp could be returned to its clamped configuration. The foregoing could be repeated any number of times.

At least the multi-function catheter shaft tool 270 (FIGS. 5A-5B), the multi-function catheter shaft tool 290 (FIGS. 6A-7C), the catheter shaft actuator 370 (FIGS. 8A-8D), the catheter shaft actuator 440 (FIGS. 9A-9C and 10A-10B), the catheter shaft actuator 492 (FIGS. 11A1-11A4), the catheter shaft actuator 532 (FIGS. 11B1-11B3), the catheter shaft actuator 532′ (FIGS. 11B4-11B5), the catheter shaft actuator 552 (FIGS. 12A1-12A4), the catheter shaft actuator 592 (FIGS. 12B1-12B3), the catheter shaft actuator 620 (FIGS. 12C1-12C4), the catheter shaft actuator 650 (FIGS. 12D1-12D5), the catheter shaft actuator 690 (FIG. 13A), the catheter shaft actuator 720 (FIG. 13B), and the catheter shaft actuator 750 (FIG. 13C) could be used with both: 1) a catheter handle having a generator cable extending therefrom (e.g., catheter handle 210 and generator cable 250, shown in FIGS. 3A-3B); and 2) a non-catheter handle housing having a generator cable extending therefrom (e.g., housing 422 and generator cable 434).

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

Any feature of any other various aspects addressed in this disclosure that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular. Moreover, any failure to use phrases such as “at least one” also does not limit the corresponding feature to the singular. Use of the phrase “at least substantially,” “at least generally,” or the like in relation to a particular feature encompasses the corresponding characteristic and insubstantial variations thereof (e.g., indicating that a surface is at least substantially or at least generally flat encompasses the surface actually being flat and insubstantial variations thereof). Finally, a reference of a feature in conjunction with the phrase “in one embodiment” does not limit the use of the feature to a single embodiment.

1. A catheter, comprising:
a catheter shaft comprising a shaft distal end; and
a catheter shaft actuator comprising an actuator distal end, wherein the catheter shaft extends distally of the actuator distal end, and wherein a distance between the shaft distal end and the actuator distal end is adjustable.

2. The catheter of claim 1, further comprising a multi-function catheter shaft tool comprising:
a tool distal end;
a tool proximal end, the catheter shaft tool defining a lumen that extends from the tool distal end to the tool proximal end, wherein the lumen is sized to accommodate the catheter shaft;
a catheter shaft straightener comprising the tool distal end;
and
a catheter shaft clamp disposable in each of a clamping configuration and a released configuration relative to the lumen,
wherein the catheter shaft straightener extends proximally from the tool distal end and at least to the actuator distal end, and wherein the catheter shaft straightener and the catheter shaft actuator are configured to be collectively rotatable.

3. The catheter of claim 1, further comprising a catheter handle, wherein the catheter shaft extends at least to a distal end of the catheter handle, wherein at least part of the catheter shaft actuator is disposed distally of the catheter handle, and wherein the catheter shaft actuator comprises:
a passage extending through the catheter shaft actuator from the actuator distal end to an actuator proximal end, wherein the catheter shaft extends entirely through the catheter shaft actuator;
a clamping actuator on an exterior of the catheter shaft actuator;
a catheter shaft clamp operatively interconnected with the clamping actuator;
a clamping configuration, relative to the catheter shaft, when the clamping actuator is in a first position;
a released configuration, relative to the catheter shaft, when the clamping actuator is in a second position, wherein the clamping actuator is movable between the first position and the second position to dispose the clamp in the clamping configuration and the released configuration, respectively;
a body;
a first tube extending distally from the body, wherein the catheter shaft extends through the first tube; and
a second tube extending proximally from the body, wherein the first tube and the second tube are spaced along a length of the catheter shaft actuator, and wherein the passage comprises an interior of the first tube and an interior of the second tube.

4. The catheter of claim 1, wherein the catheter shaft actuator comprises an actuator handle and a clamping actuator connectable with the actuator handle, and either:
wherein the actuator handle comprises an actuation surface, wherein the clamping actuator comprises a clamp, wherein the clamp extends into the actuator handle and interfaces with the actuation surface, and wherein the catheter shaft extends through the clamp of the clamping actuator and through the actuator handle; or
wherein the actuator handle comprises a clamp, wherein the clamping actuator comprises an actuation surface, wherein the clamp of the actuator handle extends into the clamping actuator and interfaces with the actuation surface, and wherein the catheter shaft extends through the clamping actuator and through the clamp of the actuator handle.

5. The catheter of claim 1, wherein the catheter shaft actuator comprises an actuator handle, wherein the actuator handle comprises first and second clamping segments that are separated by a space, wherein the catheter shaft extends through the space between the first and second clamping segments, and wherein the actuator handle further comprises a clamping actuator engageable with the first and second clamping segments.

6. The catheter of claim 1, wherein the catheter shaft actuator comprises an actuator handle, a clamping actuator, and a rotary catheter shaft clamp comprising a first clamping segment and a second clamping segment.

7. The catheter of claim 6, wherein the first clamping segment and the second clamping segment are engageable with opposite sides of the catheter shaft.

8. The catheter of claim 1, wherein the catheter shaft actuator comprises an actuator handle and a clamping actuator, wherein the clamping actuator comprises a collet rotatably connected with an outer perimeter of the actuator handle, wherein the catheter shaft extends through each of the actuator handle and the clamping actuator, wherein the collet is rotatable between at least first and second positions relative to the actuator handle, wherein the actuator handle is disposed in a clamping configuration, relative to the catheter shaft, when the collet is in the first position, and wherein the actuator handle is disposed in a released configuration, relative to the catheter shaft, when the collet is in the second position.

9. The catheter of claim 1, wherein the catheter shaft actuator comprises an actuator handle, a first clamp, and a clamping actuator, wherein the catheter shaft extends through the actuator handle, wherein the first clamp is movable along a first axis relative to the catheter shaft between a clamping configuration and a released configuration, wherein the clamping actuator is movable along a second axis between at least first and second positions to dispose the first clamp in the clamping configuration and the released configuration, respectively, wherein the first clamp clamps against the catheter shaft in the clamping configuration, and wherein the catheter shaft actuator is movable along and relative to the catheter shaft when the first clamp is in the released configuration.

10. The catheter of claim 1, wherein the catheter shaft actuator comprises an actuator handle, a first clamp, a clamping actuator interconnected with the first clamp, and a biasing member that engages at least one of the first clamp and the clamping actuator, wherein the catheter shaft extends through the actuator handle, wherein the clamping actuator is movable between at least first and second positions and the first clamp is movable between a clamping configuration and a released configuration, relative to the catheter shaft, wherein one of the clamping actuator and the biasing member disposes the first clamp in the clamping configuration, and wherein the other of the clamping actuator and the biasing member disposes the first clamp in the released configuration.

11. The catheter of claim 1, further comprising:
a telescoping catheter handle having:
a first catheter handle configuration where the telescoping catheter handle is of a first handle length and the catheter shaft is of a first working length; and
a second catheter handle configuration where the telescoping catheter handle is of a second handle length and the catheter shaft is of a second working length,
wherein the first handle length is different from the second handle length and the first working length is different from the second working length.

12. The catheter of claim 11, wherein the telescoping catheter handle comprises first and second holes that are spaced along a length dimension of the telescoping catheter handle and that correspond with the first catheter handle configuration and the second catheter handle configuration, respectively, the catheter further comprising at least one detent, wherein the at least one detent is disposed in the first hole when the telescoping catheter handle is in the first catheter handle configuration, and wherein the at least one detent is disposed in the second hole when the telescoping catheter handle is in the second catheter handle configuration.

13. The catheter of claim 1, wherein the catheter shaft actuator comprises an actuator handle, the catheter further comprising:
a shuttle movable along a length of the actuator handle between at least first and second positions that are spaced along the length of the actuator handle, wherein the catheter shaft is interconnected with the shuttle such that the shuttle and the catheter shaft are collectively movable relative to the actuator handle at least generally in an axial dimension,
wherein disposing the shuttle in the first position provides a first working length for the catheter shaft and disposing the shuttle in the second position provides a second working length for the catheter shaft, and wherein the first working length and the second working length are different.

14. The catheter of claim 13, wherein the actuator handle comprises a perimeter wall and a slot extending through the perimeter wall and extending between a first slot end and a second slot end, and wherein the first slot end defines the first position and the second slot end defines the second position.

15. The catheter of claim 13, further comprising:
a proximal shaft extending proximally from a proximal end of the actuator handle; and
a distal shaft extending distally from a distal end of the actuator handle,
wherein the catheter shaft extends through the distal shaft and the shuttle is separately disposable in each of the proximal shaft and the distal shaft.

16. The catheter of claim 1, further comprising:
an actuator handle;
a proximal sleeve extending proximally from a proximal end of the actuator handle, wherein the catheter shaft through each of the actuator handle and the proximal sleeve;
a first stop; and
a second stop,
wherein the first stop and the second stop are spaced along a dimension that the catheter shaft and the first shaft are collectively movable relative to each of the catheter handle and the proximal sleeve, and
wherein the first stop defines at least in part one catheter shaft working length configuration and the second stop defines at least in part a different catheter shaft working length configuration.

17. The catheter of claim 1, further comprising:
a first handle; and
a shaft interconnected with and movable in conjunction with the catheter shaft, wherein each of the catheter shaft and the shaft are movable relative to the first handle, wherein an outer perimeter of the shaft comprises either:
first and second recesses that are spaced along a length dimension of the shaft and a detent movable relative to the shaft and separately disposable in each of the first and second recesses, wherein the detent being in the first recess defines one catheter shaft working length configuration and the detent being in the second recess defines a different catheter shaft working length configuration; or
a shaft slot that extends along a length dimension of the shaft and a shaft clamp movable relative to the shaft and engageable with the shaft slot at a plurality of different positions along the slot, wherein the shaft clamp engaging one location along the shaft slot defines one catheter shaft working length configuration and the shaft clamp engaging a different location along the shaft slot defines a different catheter shaft working length configuration.

18. A catheter handle assembly, comprising:
a catheter handle comprising a distal end and a proximal end that are spaced along a first dimension;
a shuttle connectable with a catheter shaft, wherein the shuttle is movable along the first dimension relative to at least the distal end of the catheter handle; and
a lock interconnected with the shuttle and disposed on an exterior of the catheter handle, wherein the lock is configured to be separately disposable in a locked configuration and an unlocked configuration, wherein the shuttle is movable relative to the catheter handle when the lock is in the unlocked configuration, and wherein a position of the shuttle is fixed relative to the catheter handle when the lock is in the locked configuration.

19. A catheter, comprising:
a catheter shaft;
a catheter shaft actuator movably mounted on the catheter shaft;
a housing disposed in a fixed position relative to the catheter shaft, wherein at least part of the housing extends proximally relative to the catheter shaft actuator; and
a generator cable extending proximally from the housing, wherein a distal end section of the housing is typically disposed within a proximal end section of the catheter shaft actuator when the catheter shaft actuator is disposed in a proximal-most position relative to the catheter shaft.
